(19) United States
(12) Patent Application Publication
(10) Pub. No.: US 2025/0222107 A1
(43) Pub. Date: Jul. 10, 2025

(54) ANTI-BCMA CAR TO TARGET IMMUNE-RELATED DISORDERS, COMPOSITIONS AND METHOD THEREOF

(71) Applicants: HADASIT MEDICAL RESEARCH SERVICES AND DEVELOPMENT LTD., Jerusalem (IL); BAR ILAN UNIVERSITY, Ramat Gan (IL)

(72) Inventors: Cyrille Joseph COHEN, Petach-Tikva (IL); Polina STEPENSKY, Jerusalem (IL); Shlomit KFIR-ERENFELD, Jerusalem (IL); Nathalie ASHERIE, Jerusalem (IL); Ortal HARUSH, Or Yehuda (IL)

(21) Appl. No.: 18/837,381

(22) PCT Filed: Feb. 9, 2023
(86) PCT No.: PCT/IL2023/050142

§ 371 Date: Aug. 9, 2024

Related U.S. Application Data
(60) Provisional application No. 63/308,277, filed on Feb. 9, 2022.
(60) Provisional application No. 63/368,002, filed on Jul. 8, 2022.

(51) Int. Cl. A61K 40/42; A61K 35/17; A61K 40/11; A61K 40/31; A61P 37/02; C07K 14/705; C07K 14/725; C07K 16/28; C12N 5/0783

(52) U.S. Cl. CPC A61K 40/4215; A61K 35/17; A61K 40/11; A61K 40/31; A61P 37/02; C07K 14/7051; C07K 14/70517; C07K 14/70575; C07K 16/2878; C12N 5/0636; A61K 2239/13; A61K 2239/17; A61K 2239/21; A61K 2239/22; C12N 2510/00

(57) ABSTRACT

The present disclosure provides chimeric antigen receptor (CAR) molecule specific for B cell maturation antigen (BC-MA), compositions and methods for treating immune-related disorders, specifically, plasma cell pathologies such as multiple myeloma (MM).

### TECHNOLOGICAL FIELD

The present disclosure relates to immunotherapy. More specifically, the present disclosure relates to a chimeric antigen receptor (CAR) molecule specific for B cell maturation antigen (BCMA), compositions and methods thereof for the treatment of immune-related disorders.

### BACKGROUND ART

References considered to be relevant as background to the presently disclosed subject matter are listed below:
[1] Friedman K M, Garrett T E, Evans J W et al. Effective Targeting of Multiple B-Cell Maturation Antigen-Expressing Hematological Malignances by Anti-B-Cell Maturation Antigen Chimeric Antigen Receptor T Cells. Hum. Gene Ther 2018; 29:585-601.
[2] Carpenter R O, Evbuomwan M O, Pittaluga S et al. B-cell maturation antigen is a promising target for adoptive T-cell therapy of multiple myeloma. Clin. Cancer Res 2013; 19:2048-2060.
[3] Ali S A, Shi V, Maric I et al. T cells expressing an anti-B-cell maturation antigen chimeric antigen receptor cause remissions of multiple myeloma. Blood 2016; 128:1688-1700.
[4] Brudno J N, Maric I, Hartman S D et al. T Cells Genetically Modified to Express an AntiΓàóB-Cell Maturation Antigen Chimeric Antigen Receptor Cause Remissions of Poor-Prognosis Relapsed Multiple Myeloma. JCO 2018; 36:2267-2280.
[5] Raje N, Berdeja J, Lin Y et al. Anti-BCMA CAR T-Cell Therapy bb2121 in Relapsed or Refractory Multiple Myeloma. N Engl J Med 2019; 380:1726-1737.
[6] Cohen A D, Garfall A L, Stadtmauer E A et al. B cell maturation antigenΓàóspecific CAR T cells are clinically active in multiple myeloma. J Clin Invest 2019; 129:2210-2221.
[7] Jiang S, Jin J, Hao S et al. Low Dose of Human scFv-Derived BCMA-Targeted CAR-T Cells Achieved Fast Response and High Complete Remission in Patients with Relapsed/Refractory Multiple Myeloma. Blood 2018; 132:960.
[8] Liu Y, Chen Z, Fang H et al. Durable Remission Achieved from Bcma-Directed CAR-T Therapy Against Relapsed or Refractory Multiple Myeloma. Blood 2018; 132:956.
[9] Mailankody S, Ghosh A, Staehr M et al. Clinical Responses and Pharmacokinetics of MCARH171, a Human-Derived Bcma Targeted CAR T Cell Therapy in Relapsed/Refractory Multiple Myeloma: Final Results of a Phase I Clinical Trial. Blood 2018; 132:959.
[10] Munshi N C, Anderson L D, Jr., Shah N et al. Idecabtagene Vicleucel in Relapsed and Refractory Multiple Myeloma. N. Engl. J. Med. 2021; 384:705-716.
[11] Eisenberg V, Hoogi S, Shamul A, Barliya T, Cohen C J. T-cells “a la CAR-T(e)”—Genetically engineering T-cell response against cancer. Adv. Drug Deliv. Rev. 2019; 141:23-40.
[12] Kawalekar O U, O'Connor R S, Fraietta J A et al. Distinct Signaling of Coreceptors Regulates Specific Metabolism Pathways and Impacts Memory Development in CAR T Cells. Immunity. 2016; 44:380-390.
[13] Watanabe N, Bajgain P, Sukumaran S et al. Fine-tuning the CAR spacer improves T-cell potency. Oncoimmunology. 2016; 5:e1253656.
[14] Ying Z, Huang X F, Xiang X et al. A safe and potent anti-CD19 CAR T cell therapy. Nat. Med. 2019; 25:947-953.

Acknowledgement of the above references herein is not to be inferred as meaning that these are in any way relevant to the patentability of the presently disclosed subject matter.

### BACKGROUND OF THE INVENTION

Multiple myeloma (MM) develops in the bone marrow by clonal expansion of malignant plasma cells and is associated with excessive production of monoclonal immunoglobulins in blood and urine. Despite the significant progress achieved in the treatment of MM, this disease remains incurable with a poor prognosis in relapsed/refractory (R/R) patients. Therefore, developing novel therapeutic approaches to target multiple myeloma are required. Following the major success of CD19-targeted CAR therapy to hematological malignancies and its FDA approval in 2017, new potential targets for CAR therapy are required. In this regard, B cell maturation antigen (BCMA) is a cell surface protein belonging to the tumor necrosis factor receptor (TNFR) superfamily. Following its interaction with APRIL and BAFF, BCMA can promote B-cell survival and proliferation. Apart from mature B lymphocytes and plasma cells, BCMA is highly expressed in most cases of MM, making it an attractive target for CAR therapy [1].

Kochenderfer and colleagues tested an anti-BCMA CAR in a clinical trial which resulted in 20% objective response rate in the patient group treated with 0.3-3×10⁶ CAR T-cells/kg [2]. A higher overall response rate (ORR) (81%) was achieved in the patient group who received the highest dose of CAR T-cells (9×10⁶ cells/kg) [3, 4]. Several recent trials demonstrate the potential of anti-BCMA CAR approach in promising Phase I/II studies, reaching up to 80% ORR with deep and durable responses in heavily pretreated patients with R/R multiple myeloma [5-10]. Based on the impressive results of a Phase II trial published in NEJM, the FDA and EMA approved the Idecabtagene Vicleucel (Ide-cel or bb2121) for the treatment of R/R MM patients. The additional CAR T cell product, showing very encouraging results is Ciltacabtagene autoleucel (Cilta-cel), which incorporates two anti-BCMA single heavy chain domains and a 4-1BB costimulatory domain. Few clinical trials using this construct had demonstrated ORR more than 80% without excessive toxicity in terms of cytokine release syndrome or neurotoxicity.

It is reasonable to surmise that the need for high doses of CAR T-cells to achieve responses [10] and prevent relapse might be correlated to low affinity of the targeting moiety, poor antigen expression, lack of T-cell persistence over time and/or to reduced CAR expression and stability [11]. Thus, CAR molecule might be modulated to enhance engineered T-cell function.

CAR molecules consist of two essential moieties: an extracellular binding domain and a signaling domain. The binding domain, usually composed of a single-chain fragment variable (scFv) targeting a designed antigen, whereas the signaling domain, usually composed of co-stimulatory moiety (CD28 and/or 4-1BB), along with CD3ζ, facilitates T-cell activation. Additionally, the type of co-stimulation can greatly facilitate CAR T-cell persistence and metabolic activity (such as seen in 4-1BB-based CARs), while CD28 promotes more potent but short-lived responses [12]. Connecting both CAR moieties, the hinge and transmembrane domains provide proper linkage and flexibility of the CAR molecule, influencing CAR-T-cell function, construct stability and expression. The choice of a specific hinge domain for CARs is often chosen according to the target ligand proximity to the cell membrane.

Being a crucial determinant for its function, CAR composition and structure is generally optimized empirically. For example, a study of Zhang et al. suggested that incorporating the transmembrane domains of CD8 or CD28 instead of that of CD3ζ may result in greater CAR surface expression. In another study, Smith et al. showed that the use of a longer hinge domain increased CAR potency and specificity. It was also shown how optimization of CAR design and co-stimulation may reduce activation induced cell death (AICD) and lead to improved CAR expression over time. Beyond specificity, the hinge composition may also influence tonic signaling and improved in vivo activity; it was shown that modifications in IgG-based hinge improved in vivo activity and reduced tonic signaling effects mediated by CH2CH3 domains located in IgG hinges [13]. It was also recently demonstrated that modifications in CD8α hinge and TM domains result in higher anti-apoptotic molecule expression in CAR-T-cells and reduced cytokine secretion, along with retained cytotoxic function [14]. A recent retrospective analysis of anti-CD19 CAR-T-based clinical studies, further support the idea that the structural composition of CAR domains, beyond scFv and the co-stimulatory moieties, impacts clinical outcomes and related toxicities. For example, it has been suggested that CD28 hinge-TM-based CAR displayed higher clinical efficacy and severe cytotoxicity, compared to CD8 hinge-TM-based CARs.

There is therefore need in the art to provide improved BCMA-CAR-T molecules, that are empirically evaluated for optimal configuration and exhibit increased specificity and high in vitro and long-term in vivo efficacy. These needs are addressed by the present specification.

### SUMMARY OF THE INVENTION

A first aspect of the present disclosure relates to a chimeric antigen receptor (CAR) molecule comprising the following components: (i) at least one target-binding domain; wherein at least one of said target binding domain specifically recognizes and binds B cell maturation antigen (BCMA); (ii) at least one hinge and at least one transmembrane domain derived from the Cluster of Differentiation 8 α (CD8α) protein (also known as T-Cell Surface Glycoprotein CD8 Alpha Chain). It should be noted that the hinge region of the domain comprises the amino acid sequence as denoted by SEQ ID NO:9, and any fragments, derivatives and variants thereof. The CAR molecule further comprises (iii) at least one intracellular T cell signal transduction domain. More specifically, this domain comprises at least one domain of tumor necrosis factor (TNF) receptor family member, and optionally, at least one domain of a T cell receptor (TCR) molecule. In some embodiments, the CAR T molecule of the present disclosure comprises the amino acid sequence as denoted by any one of SEQ ID NO: 1, or any fragments and derivatives thereof, for example, the CAR T molecule that comprises the amino acid sequence as denoted by SEQ ID NO: 40.

A further aspect of the present disclosure relates to a nucleic acid molecule comprising at least one nucleic acid sequence encoding at least one CAR molecule, or any cassette, vector or vehicle comprising said nucleic acid molecule. In some embodiments, such encoded CAR molecule comprises the following components. First (i), at least one target-binding domain; wherein at least one of said target binding domain specifically recognizes and binds BCMA; second (ii), at least one hinge and at least one transmembrane domain derived from the CD8α protein. It should be noted that the hinge region of the domain comprises the amino acid sequence as denoted by SEQ ID NO:9 and any fragments, derivatives and variants thereof. The CAR molecule further comprises as a third component (iii), at least one intracellular T cell signal transduction domain. More specifically, this domain comprises at least one domain of TNF receptor family member, and optionally, at least one domain of a TCR molecule.

A further aspect of the present disclosure relates to a gene editing system comprising:
First (i), at least one nucleic acids molecule as defined by the present disclosure, or any cassette, vector or vehicle comprising the at least one nucleic acid molecule; and
Second (ii), at least one gene editing component or a nucleic acid sequence encoding the gene editing component. In some specific embodiments, the at least one nucleic acids molecule of the gene editing system provided by the present disclosure encodes at least one CAR molecule comprising the following components: (i), at least one target-binding domain; wherein at least one of said target binding domain specifically recognizes and binds BCMA; (ii), at least one hinge and at least one transmembrane domain derived from the CD8α protein. It should be noted that the hinge region of the domain comprises the amino acid sequence as denoted by SEQ ID NO:9, and any fragments, derivatives and variants thereof; and (iii), at least one intracellular T cell signal transduction domain. More specifically, this domain comprises at least one domain of TNF receptor family member, and optionally, at least one domain of a TCR molecule.

A further aspect of the present disclosure relates to a genetically engineered cell of the T cell lineage expressing at least one CAR molecule, or any population of cells comprising at least one of the genetically modified cell/s disclosed herein. More specifically, in some embodiments, the CAR expressed by the engineered cells comprises the following components: First (i), at least one target-binding domain; wherein at least one of said target binding domain specifically recognizes and binds BCMA; second (ii), at least one hinge and at least one transmembrane domain derived from the CD8α protein. It should be noted that the hinge region of the domain comprises the amino acid sequence as denoted by SEQ ID NO:9, and any fragments, derivatives and variants thereof. The CAR molecule further comprises as a third component (iii), at least one intracellular T cell signal transduction domain. More specifically, this domain comprises at least one domain of TNF receptor family member, and optionally, at least one domain of a TCR molecule.

A further aspect of the present disclosure relates to a composition comprising at least one CAR molecule, any nucleic acid molecule comprising at least one nucleic acid sequence encoding said CAR molecule, or any, cassette, vector, vehicle or gene editing system comprising the nucleic acid molecule, any host cell expressing said CAR molecule, and/or any genetically engineered cell of the T lineage expressing said CAR or population of cells comprising at least one said genetically engineered cell of the T lineage. More specifically, such CAR molecule comprises the following components. First (i), at least one target-binding domain; wherein at least one of said target binding domain specifically recognizes and binds BCMA; second (ii), at least one hinge and at least one transmembrane domain derived from the CD8α protein. It should be noted that the hinge region of the domain comprises the amino acid sequence as denoted by SEQ ID NO:9, and any fragments, derivatives and variants thereof. The CAR molecule further comprises as a third component (iii), at least one intracellular T cell signal transduction domain. More specifically, this domain comprises at least one domain of TNF receptor family member, and optionally, at least one domain of a TCR molecule.

It should be noted that the composition of the present disclosure further comprises according to optional embodiments, at least one of pharmaceutically acceptable carrier/s, diluent/s, excipient/s and additive/s.

A further aspect of the present disclosure relates to a method for treating, preventing, ameliorating, inhibiting or delaying the onset of an immune-related disorder in a mammalian subject. In some embodiments, the method comprises the step of administering to the subject an effective amount of at least one of:
(a) at least one nucleic acid molecule encoding least one CAR molecule; (b) at least one cassette, vector vehicle or gene editing system comprising the nucleic acid molecule of (a); (c) at least one cell (specifically, cell of the T linage) expressing the CAR, or a population of such cells; and (d) a composition comprising at least one of (a), (b) and (c). More specifically, in some embodiments of the disclosed methods, such e CAR molecule comprises the following components. First (i), at least one target-binding domain; wherein at least one of the target binding domain specifically recognizes and binds BCMA; second (ii), at least one hinge and at least one transmembrane domain derived from the CD8α protein. It should be noted that the hinge region of the domain comprises the amino acid sequence as denoted by SEQ ID NO:9, and any fragments, derivatives and variants thereof. The CAR molecule further comprises as a third component (iii), at least one intracellular T cell signal transduction domain. More specifically, this domain comprises at least one domain of TNF receptor family member, and optionally, at least one domain of a TCR molecule.

A further aspect of the present disclosure relates to an effective amount of at least one of:
(a) at least one nucleic acid molecule encoding least one CAR molecule; (b) at least one cassette, vector vehicle or gene editing system comprising said nucleic acid molecule of (a); (c) at least one cell specifically, a cell of the T linage, expressing the CAR molecule, or a population of these cells; and (d) a composition comprising at least one of (a), (b) and (c); for use in a method for treating, preventing, ameliorating, inhibiting or delaying the onset of a of an immune-related disorder in a mammalian subject. In some embodiments of the discussed use, such e CAR molecule comprises the following components. First (i), at least one target-binding domain; wherein at least one of said target binding domain specifically recognizes and binds BCMA; second (ii), at least one hinge and at least one transmembrane domain derived from the CD8α protein. It should be noted that the hinge region of the domain comprises the amino acid sequence as denoted by SEQ ID NO:9, and any fragments, derivatives and variants thereof. The CAR molecule further comprises as a third component (iii), at least one intracellular T cell signal transduction domain. More specifically, this domain comprises at least one domain of TNF receptor family member, and optionally, at least one domain of a TCR molecule.

In yet some further aspect, the present disclosure provides a method for targeted activation of a cell of the T lineage against a target cell expressing the BCMA protein and/or a tissue comprising the target cell. More specifically, the method comprising the step of contacting the cell of the T linage with an effective amount of at least one of:
(a) at least one nucleic acid molecule encoding least one CAR molecule; (b) at least one cassette, vector vehicle or gene editing system comprising said nucleic acid molecule of (a); and (c) a composition comprising at least one of (a) and (b); More specifically, such CAR molecule comprises the following components. First (i), at least one target-binding domain; wherein at least one of said target binding domain specifically recognizes and binds BCMA; second (ii), at least one hinge and at least one transmembrane domain derived from the CD8α protein. It should be noted that the hinge region of the domain comprises the amino acid sequence as denoted by SEQ ID NO:9, and any fragments, derivatives and variants thereof. The CAR molecule further comprises as a third component (iii), at least one intracellular T cell signal transduction domain. More specifically, this domain comprises at least one domain of TNF receptor family member, and optionally, at least one domain of a TCR molecule.

These and other aspects of the present disclosure will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

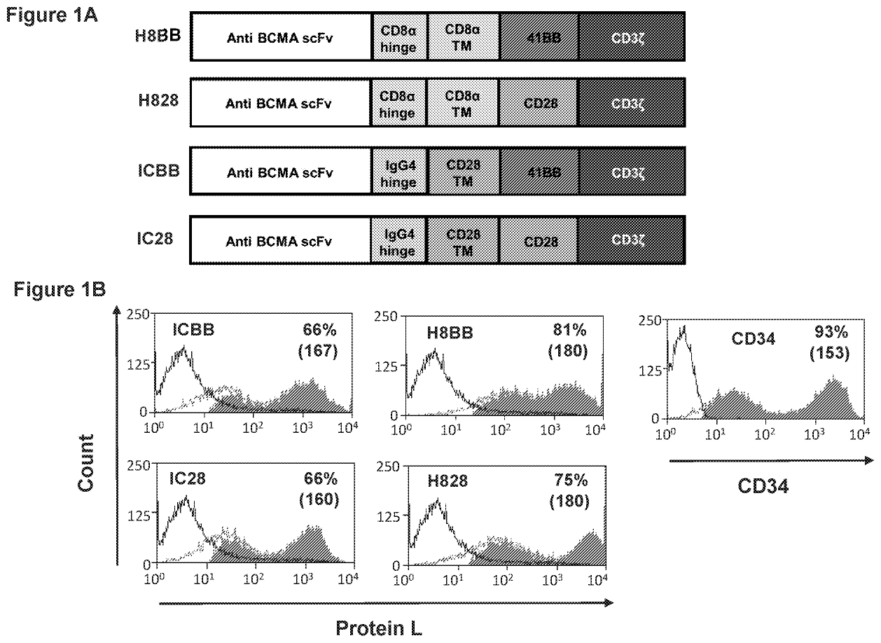

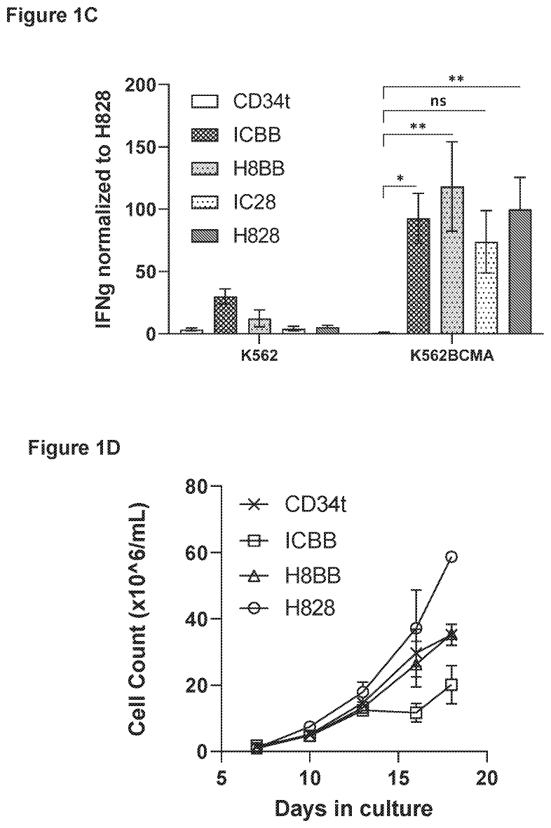

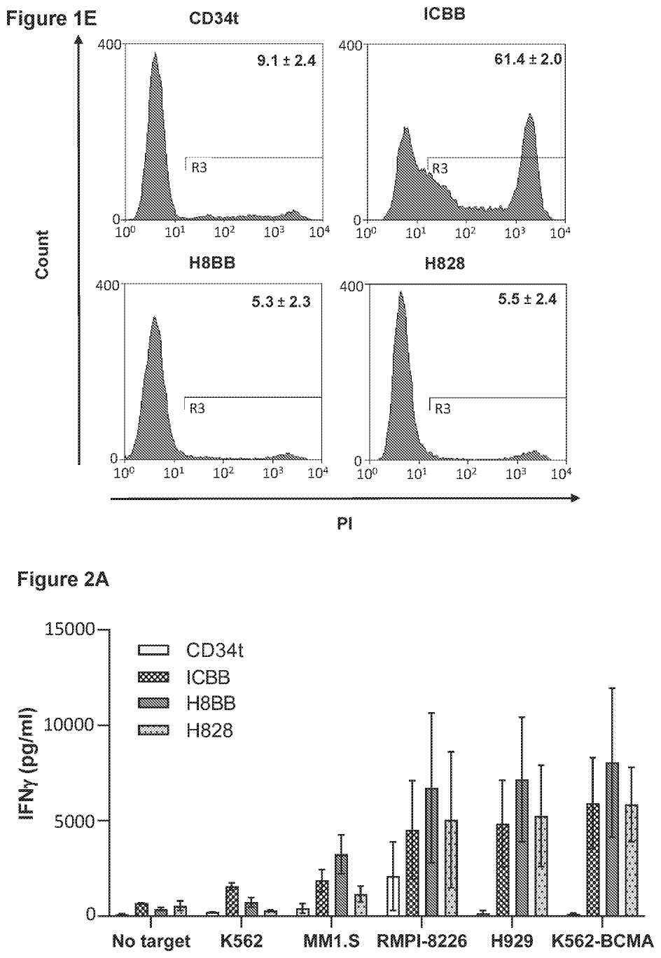

### FIG. 1A-1E. Characterization of Anti-BCMA Based Chimeras

FIG. 1A. Schematic representation of the different designs of anti-BCMA chimeric receptors described in the Materials and Methods section.

FIG. 1B. Anti-CD3-activated human primary PBMCs were transduced with the different anti-BCMA chimeric antigen receptor (CAR) or with truncated CD34 (CD34t, control gene), as indicated. These cells were stained using Protein L or anti-CD34 antibody, respectively. Transgene expression was assessed by flow cytometry. The dotted line represents the staining of the mock-transduced control. The percentage of positive cells and the mean fluorescence intensity (MFI) (in brackets) are shown. The graph represents the average of 4 different donors and the difference in the staining between the population transduced with BCMA-CARs and the control population was found statistically significant (p<0.05; calculated using a Student's paired t-test).

FIG. 1C. 10⁵ transduced cells were co-cultured in a 96-well plate with either 10⁵ K562-BCMA or K562 cells (negative control) for 16 h. Interferon γ (IFNγ) secreted in the coculture supernatant was measured by enzyme-linked immunosorbent assay (ELISA). These results are representative of 3 independent experiments.

FIG. 1D. Cell concentration was determined for BCMA-CAR T-cells and control T-cells every 2-3 days for 3 weeks. These results are representative of 3 independent experiments with different donors (ICBB vs. H8BB/H828, P<0.05; by two-way ANOVA).

FIG. 1E. Cell viability was assessed 3 weeks following transduction. BCMA-CAR T cells were stained with propidium iodide (PI) and analyzed by flow cytometry, ICBB vs. H8BB/H828, p<0.001, by paired Student's t-test).

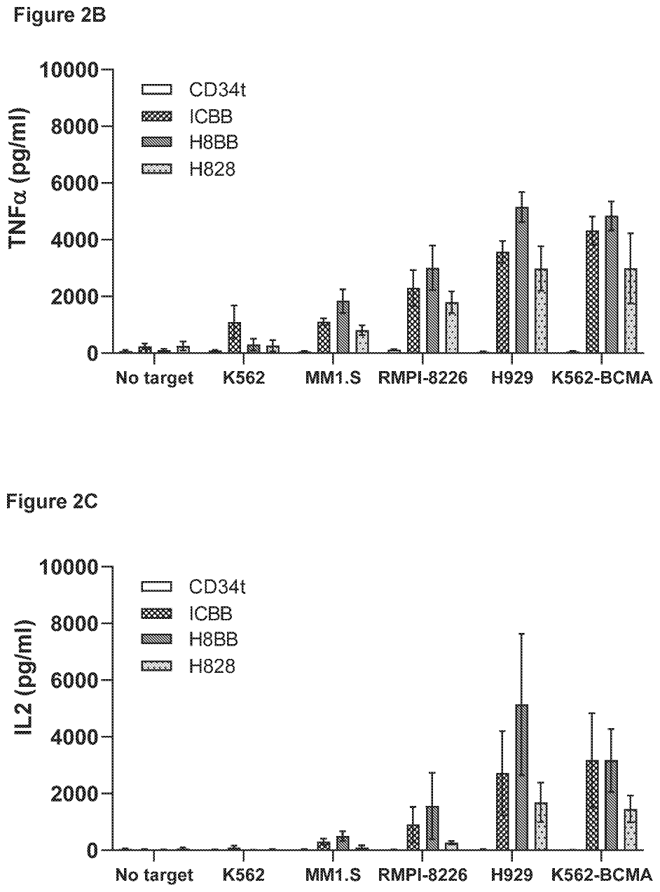

### FIG. 2A-2C. Anti-Tumor Function of BCMA-CAR-T Cells

FIG. 2A. OKT3-activated human primary B-cell maturation antigen (PBMC) were transduced with a retroviral vector encoding either ICBB-, H828-, H8BB-, and truncated CD34 (CD34t control). Transduced T cells were co-cultured with different tumor lines as indicated. Interferon γ (IFNγ) secreted in the co-culture supernatant was measured by enzyme-linked immunosorbent assay (ELISA). These results are presented as mean+standard error of the mean (SEM) (n=4, with 3 different donors; normalized to the activity of positive control CAR H828 against K562-BCMA with an average secretion of 5928 μg/ml). The difference between H8BB and H828 was found statistically significant (p=0.00098; calculated using a paired Student's t-test).

FIG. 2B-2C. Similarly, these cells were co-cultured with the indicated target T cells and TNFα (FIG. 2B) and IL2 (FIG. 2C) concentrations secreted in the culture supernatant were determined by ELISA. These results are presented as mean+SEM (n=3, with 3 different donors; normalized to the activity of positive control CAR H828 against K562-BCMA with an average secretion of 1453 μg/ml of IL2 and 2990 μg/ml of TNFα). The difference between H8BB and H828 was found statistically significant (for IL2 p=0.01 and for TNF-p=3.7×10⁻⁵; calculated using a paired Student's t-test).

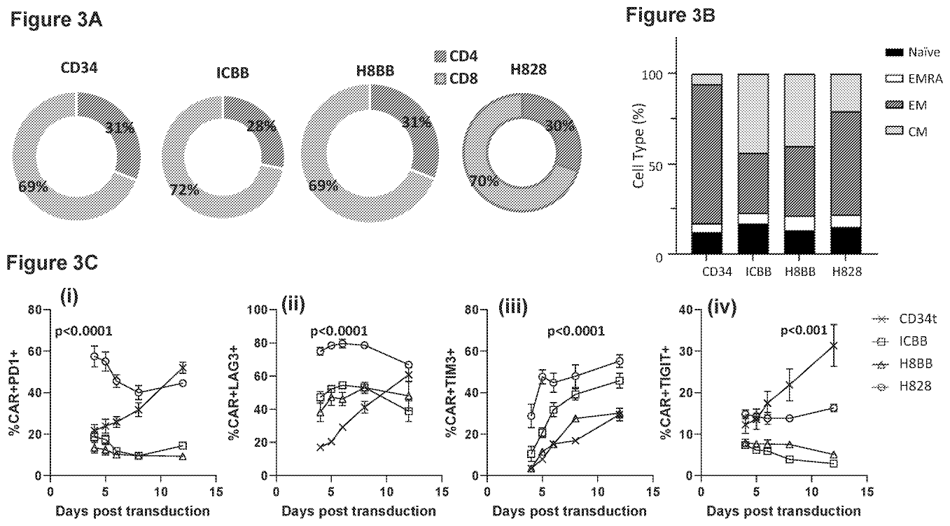

### FIG. 3A-3C. Phenotypic Characterization of BCMA-CAR Transduced T Cells

FIG. 3A. CD4/CD8 ratio of different anti-BCMA chimeras. CAR-transduced T-cells or control T-cells were stained with anti-CD4 and CD8 antibodies and analyzed by flow cytometry on day 7. These results are representative of 4 independent experiments with different donors. No significant difference was observed between the different groups.

FIG. 3B. Memory phenotype of T cells transduced with different BCMA chimeras. Three weeks following transduction, CAR-transduced T cells or control T cells were stained with anti-CD45RO and CCR7 antibodies and analyzed by flow cytometry. These results are representative of the mean of 4 independent experiments with different donors (n=4).

FIG. 3C(i)-3C(iv). BCMA CAR T cells in culture were monitored for the expression of exhaustion markers PD-1 (FIG. 3Ci), LAG-3 (FIG. 3Cii), TIM-3 (FIG. 3Ciii) and TIGIT (FIG. 3Civ) over time (as indicated).

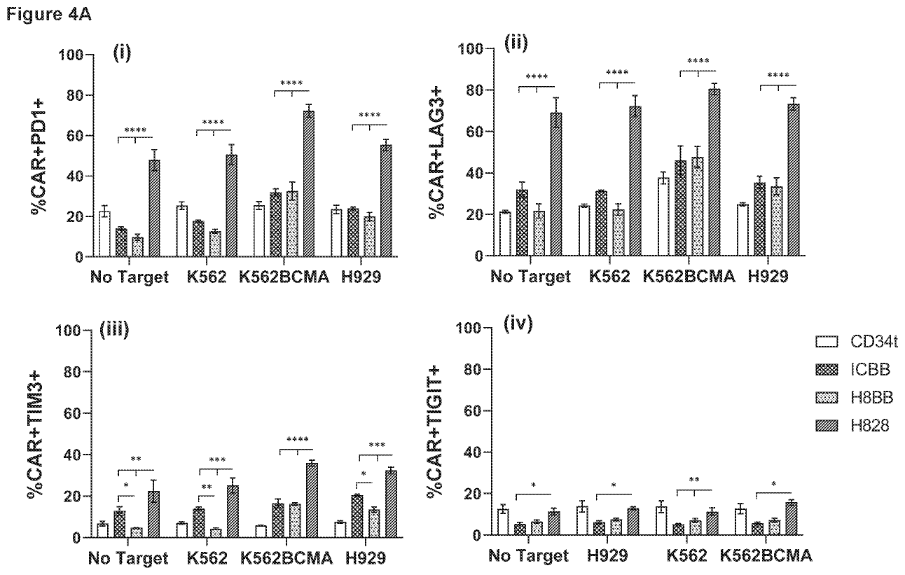

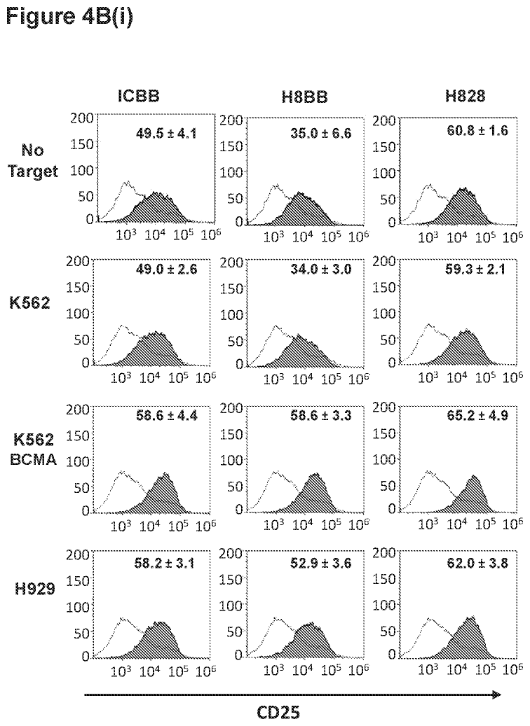

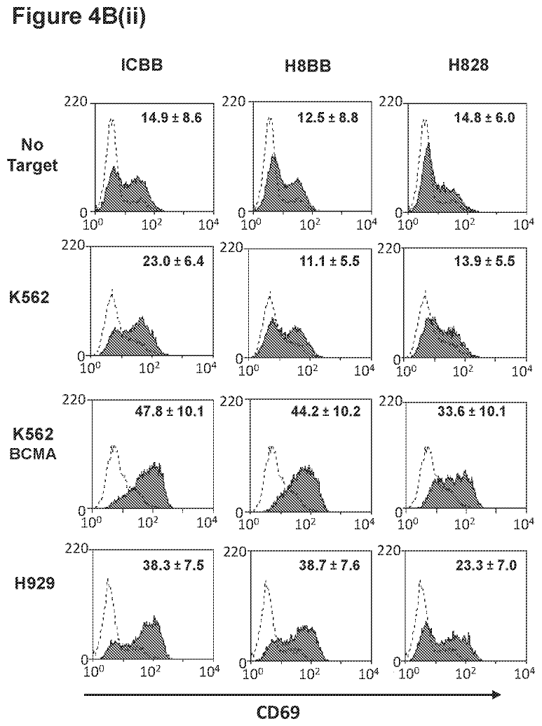

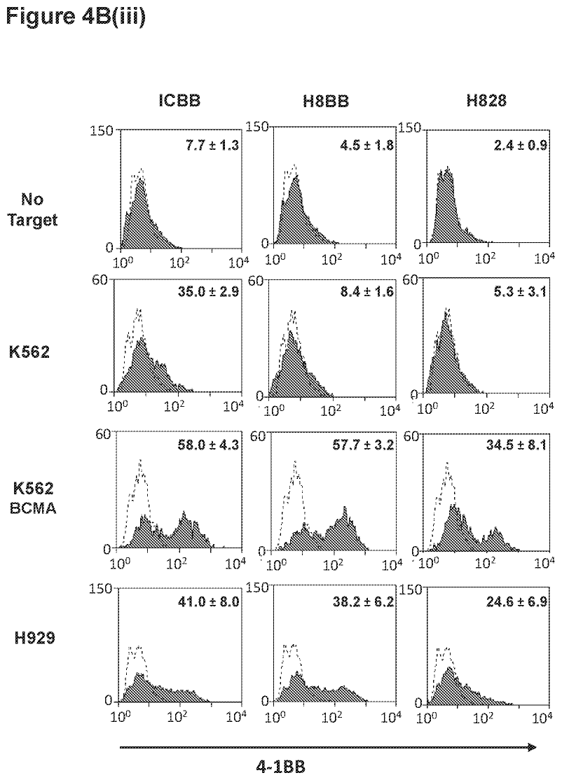

### FIG. 4A-4B. Exhaustion and Activation Profile of BCMA CAR T Cells

FIG. 4A(i)-4A(iv). BCMA-CAR-T cells were co-cultured for 24 hours in the presence of the H929-MM cell line or BCMA-overexpressing K562 (K562-BCMA), or K562 cell line in T cell medium without IL-2 at an effector to target (E:T) ratio of 10:1, in T-cell medium without IL-2, at 37° C. Following incubation, cells were washed, stained at 4° C. for 20 minutes with a mixture of the fusion protein/antibodies (as indicated), to assess the expression of PD-1 (FIG. 4Ai), LAG-3 (FIG. 4Aii), TIM-3 (FIG. 4Aiii) and TIGIT (FIG. 4Aiv) by flow cytometry (gated on BCMA.CAR+). These results are representative of 3 different experiments with different donors. Data is represented as mean values±standard error of the mean (SEM).

FIG. 4B(i)-4B(iii). CAR-transduced T-cells or CD34t cells were co-cultured with BCMA-positive targets, as indicated. Following an overnight or 4-hour co-culture (for CD69), cells were analyzed by flow cytometry for the expression of CD25 (FIG. 4B(i)), CD69 (FIG. 4B(ii)) and 4-1BB (FIG. 4B(iii)). Cells were gated on the CD8⁺ population for 4-1BB and CD69 expression, and on CAR+ cells for CD25 expression. The mean percentage of positive cells ±SEM is indicated on histograms overlay. Grey filled histograms represent CAR-T cells staining, and the dotted line histograms represent the staining of the CD34t control cells. These results are representative of 3 independent experiments with 3 different donors.

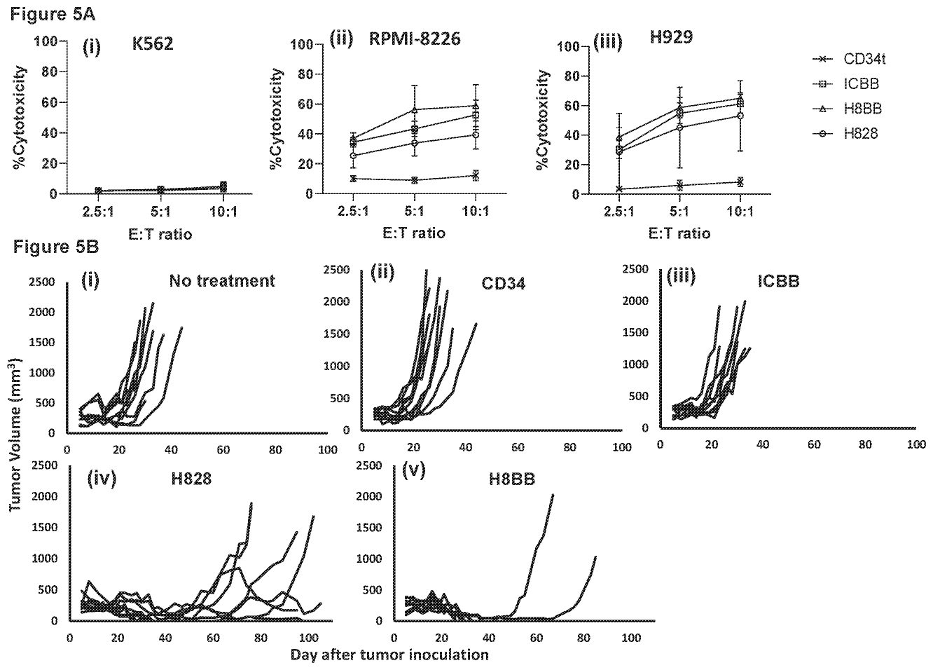

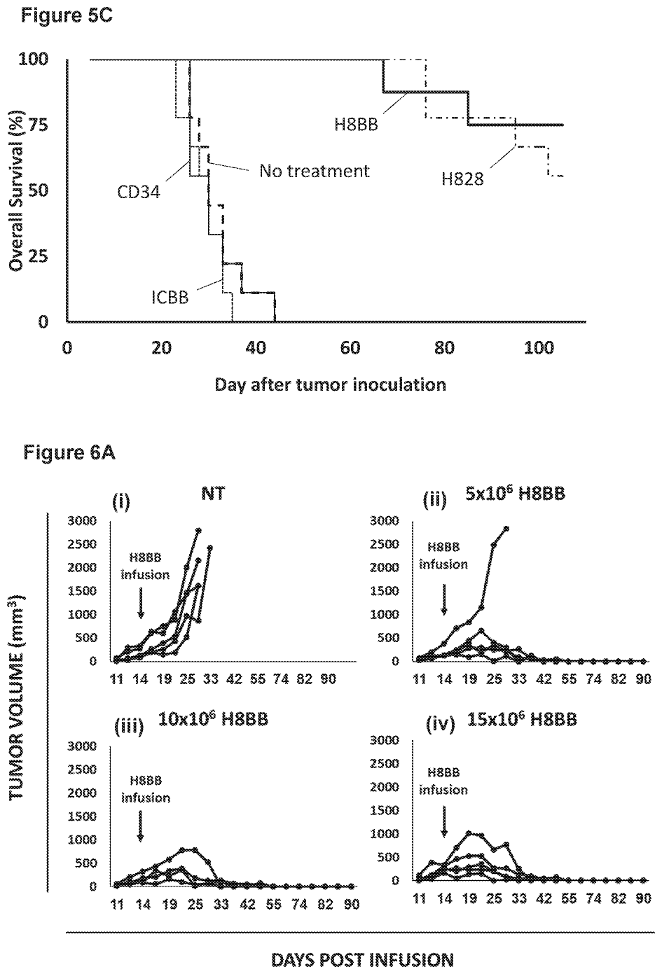

### FIG. 5A-5C. BCMA Chimeras Mediate Anti-Tumor Cytotoxic Activity In Vitro and In Vivo

FIG. 5A(i)-5A(iii). BCMA CARs or CD34t (control) transduced T cells were co-cultured with CFSE-labeled tumor cells at the indicated effector to target (E:T) ratios. After 4 hours, propidium iodide (PI) was added and the cells were analyzed by flow cytometry. Cytotoxicity was calculated based on the proportion of CFSE+/PI+ population out of the total CFSE+ population. These results are presented as mean±standard error of the mean (SEM) of 3 independent experiments with 3 different donors (FIG. 5A(i) K562; FIG. 5A(ii) RPMI-8226; and FIG. 5A(iii) H929), and the difference between the BCMA-CARs and CD34t populations was found statistically significant (p<0.05, calculated using a Student's paired t-test).

FIG. 5B(i)-5B(v). In vivo function of BCMA-CAR T-cells. NSG xenografts (n=8, per group) were inoculated H929 myeloma cells. One week following tumor inoculation, the mice were intravenously injected either with 15×10⁶ BCMA-CAR+(FIG. 5B(iii) ICBB, FIG. 5B(iv) H828, or FIG. 5B(v) H8BB), or with CD34t-transduced control cells (FIG. 5B(ii)), or with no treatment (FIG. 5B(i)). Tumor volume was measured in a blinded fashion using a caliper and calculated using the following formula: (D×d²)×Π/6, where D is the largest tumor diameter and d its perpendicular one.

FIG. 5C. Overall survival analysis. The difference in the average survival of the H828 and H8BB groups compared to the no-treatment or control groups was found statistically significant (p<1e⁻⁴; using a LogRank analysis).

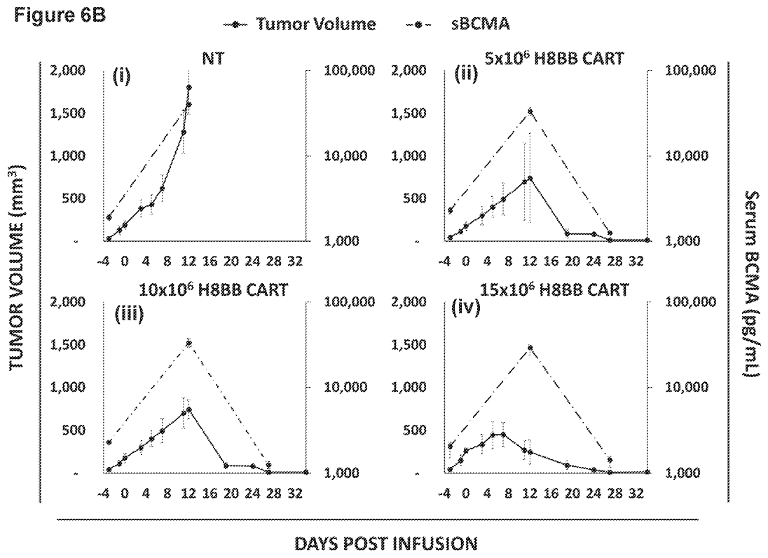

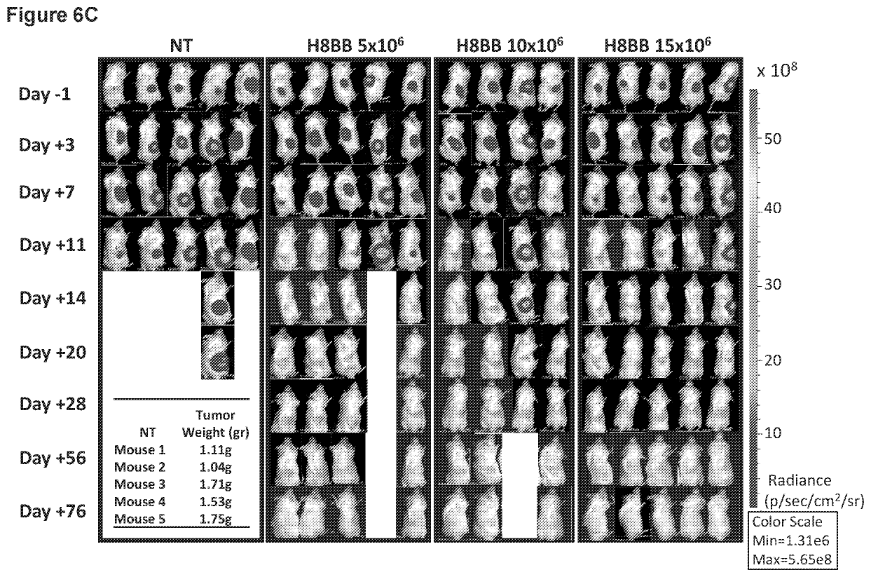

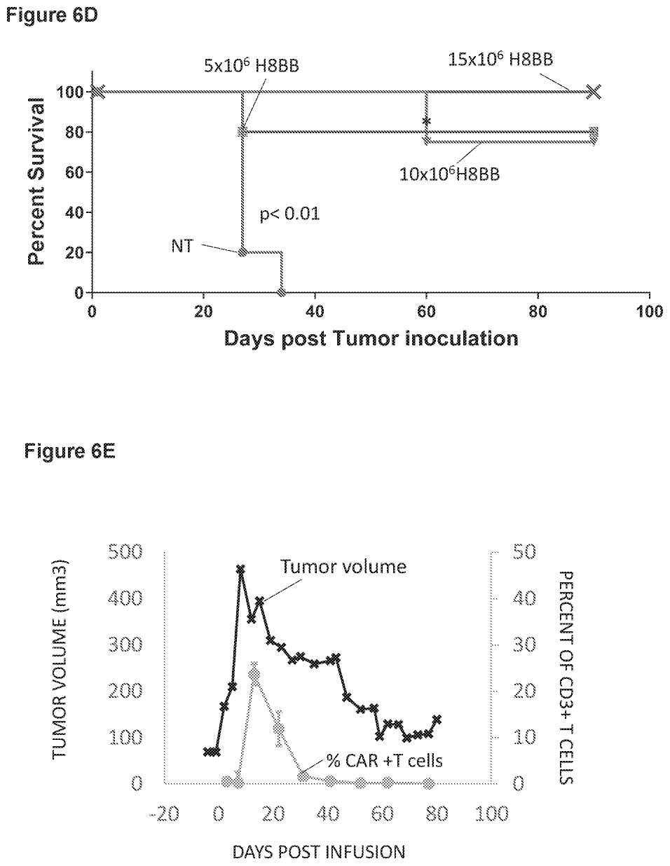

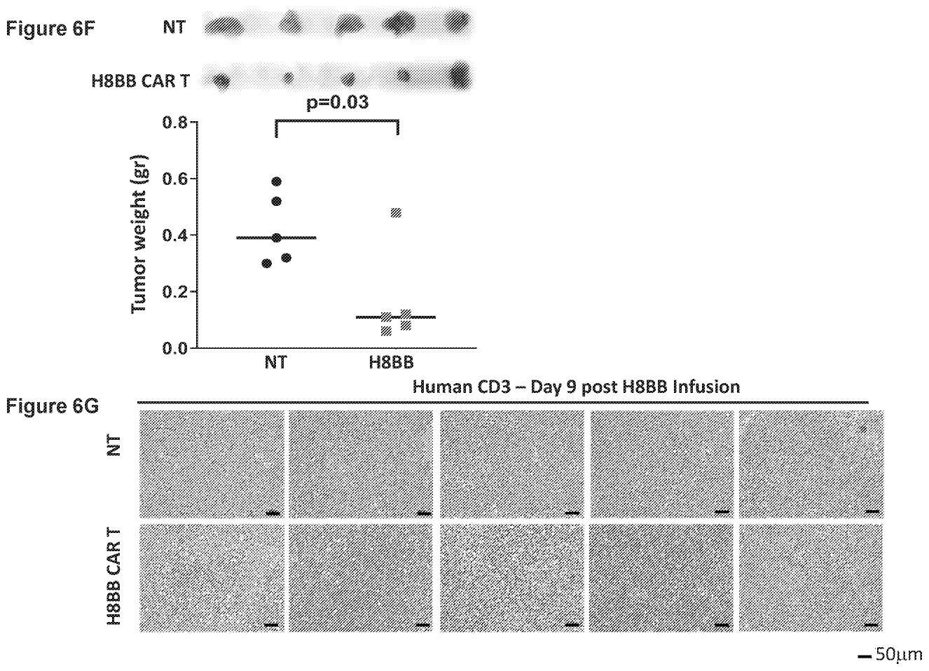

FIG. 6A-6G. Anti-Myeloma Effect of H8BB CART Cells; CAR+-T Cells Persistence in Mice is Associated with the Elimination of NCI-H929 Multiple Myeloma Tumor

FIG. 6A(i)-6A(iv). NSG mice (4-5 animals per group) with an average of approximately 200 mm³ subcutaneous NCI-H929 tumors per treatment group received a single intravenous (i.v.) administration of 15×10⁶ non-transduced (NT) control cells (FIG. 6A(i)), or 5 (FIG. 6A(ii)), 10 (FIG. 6A(iii)) or 15×10⁶ (FIG. 6A(iv)) H8BB-CAR+ T cells/mouse, respectively. Tumor size was measured by calipers twice weekly by personnel blinded to treatment conditions.

FIG. 6B(i)-6B(iv). Serum B-cell maturation antigen (BCMA) protein levels assessed by enzyme-linked immunosorbent assay (ELISA) (dashed line) were plotted with corresponding tumor volume measurements (black line), for 15×10⁶ non-transduced (NT) control cells (FIG. 6B(i)), or 5 (FIG. 6B(ii)), 10 (FIG. 6B(iii)) or 15×10⁶ (FIG. 6B(iv)) H8BB-CAR+ T cells/mouse, respectively. Error bars show standard error of the mean (SEM).

FIG. 6C. Myeloma development was monitored by bioluminescence imaging (BLI). BLI measurement in photons per second per cm² per steradian (p/s/cm²/sr) was translated to color to indicate disease activity in the mice by the legend shown. The weight of the tumors that were excised from NSG xenografts in the NT group is indicated in the table, to show that BLI reduction at this time was rather due to tumor necrosis than to a reduction in the tumor size.

FIG. 6D. Kaplan-Meier survival curves of study shown in (FIGS. 6A and 6C); * represents NSG mouse that was found dead at Day 60 post tumor inoculation, without any apparent relation to multiple myeloma.

FIG. 6E. 25 μL of blood were collected from the tail vein, lysed with IOTEST 3 Lysing Solution (Beckman Coulter) for 10 minutes and stained with a mixture of fluorescent recombinant human B-cell maturation antigen (BCMA) protein, anti-CD3, anti-CD8 and anti-CD4. The percent of H8BB CAR T cells in NSG blood (% of CD3+BCMA.CAR+ cells) was assessed by flow cytometry.

FIG. 6F. Average weight of the tumors excised from NCI-H929 multiple myeloma (MM) xenografts at Day 9 post T cell infusion. H8BB CAR T xenograft tumors are shown in the bottom panel; non-transduced (NT) xenograft control tumors are shown in the upper panel.

FIG. 6G. Infiltration of tumor tissue (depicted in FIG. 6F, upper panel) by CD3+ T cells was assessed by immunohistochemistry using anti-human CD3 antibody by Day 9 post H8BB CAR or NT T cell infusion. Marker bar represents 50-μm. Note: FIG. 6A-6D and 6E-6G refer to experiments performed on different cohorts of mice.

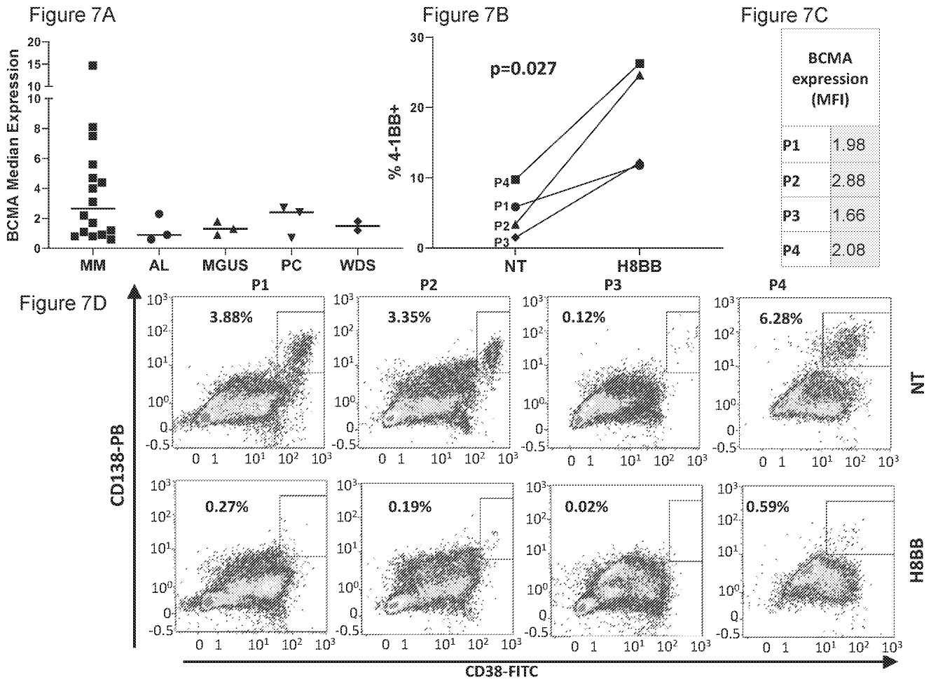

FIG. 7A-7D. H8BB CAR T cells target plasma cells of Multiple Myeloma patients FIG. 7A. Bone marrow (BM) aspirates of multiple myeloma (MM), amyloidosis (AL), monoclonal gammopathy of undetermined significance (MGUS), plasmacytoma (PC) and Walden-ström's macroglobulinemia (WDS) patients were assessed for B-cell maturation antigen (BCMA) expression by flow cytometry. The graph represents BCMA median expression in the bone marrow of patients, gated on CD38⁺⁺CD138⁺⁺ plasma cells. n=3 (AL, circles), N=16 (MM, squares), n=3 (MGUS, up triangles), n=3 (PC, down triangles), and n=2 (WDS, lozenges). Error bars show standard error of the mean (SEM).

FIG. 7B. Activation of CD3+ T cells was determined by assessing the expression of CD137 (4-1BB) by flow cytometry. H8BB CAR T and non-transduced (NT) T cells were incubated in the presence of bone marrow-derived mononuclear cells (BM-MCs) overnight. Statistical analysis was based on a Student's paired t-test.

FIG. 7C. Mean fluorescence intensity (MFI) of BCMA expression on the targeted CD38++CD138++ plasma cell population of patients P1 to P4 (p=0.01, by Student's t-test).

FIG. 7D. The presence of CD38⁺⁺CD138⁺⁺ plasma cells of MM-patients following co-culture with H8BB CAR T cells or NT control cells was assessed by flow cytometry (P1 to P4).

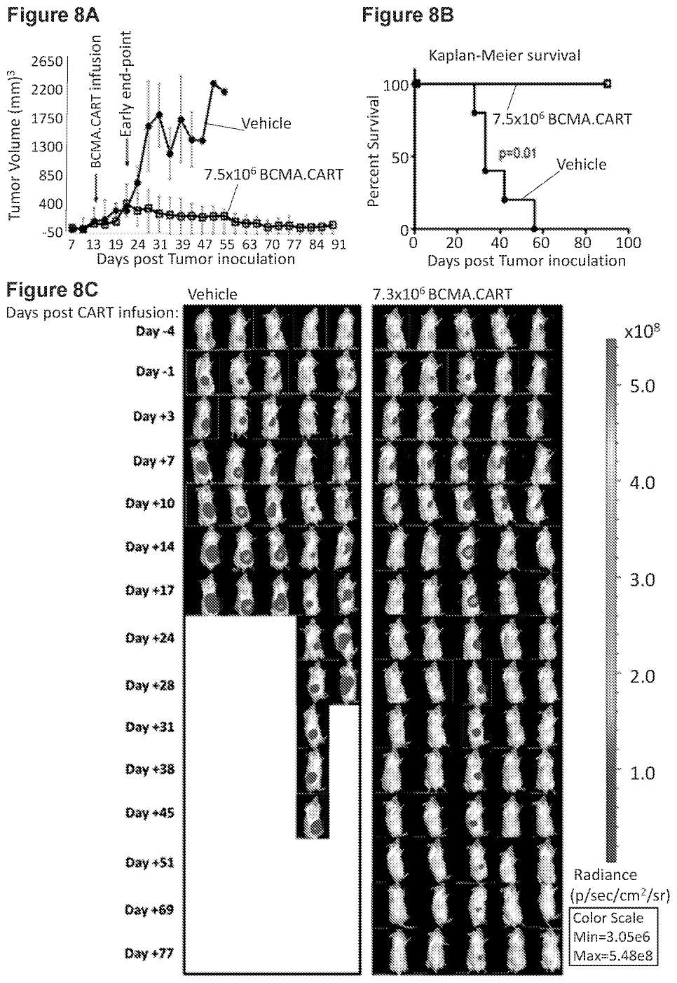

### FIG. 8A-8C. Animal Model

FIG. 8A. NSG mice (N=5 animals per group) with approximately 100 mm3 subcutaneous NCI-H929 tumors received a single intravenous (i.v.) administration of 7.5×10⁶ CAR+ T cells/mouse at Day 11 post tumor inoculation. Tumor size was measured by caliper twice weekly by personnel blinded to treatment conditions.

FIG. 8B. Kaplan-Meier survival curves of study.

FIG. 8C. Myeloma development was monitored by bioluminescence imaging (BLI). BLI measurement in photons per second per cm2 per steradian (p/s/cm2/sr) was translated to color to indicate disease activity in the mice by the legend shown.

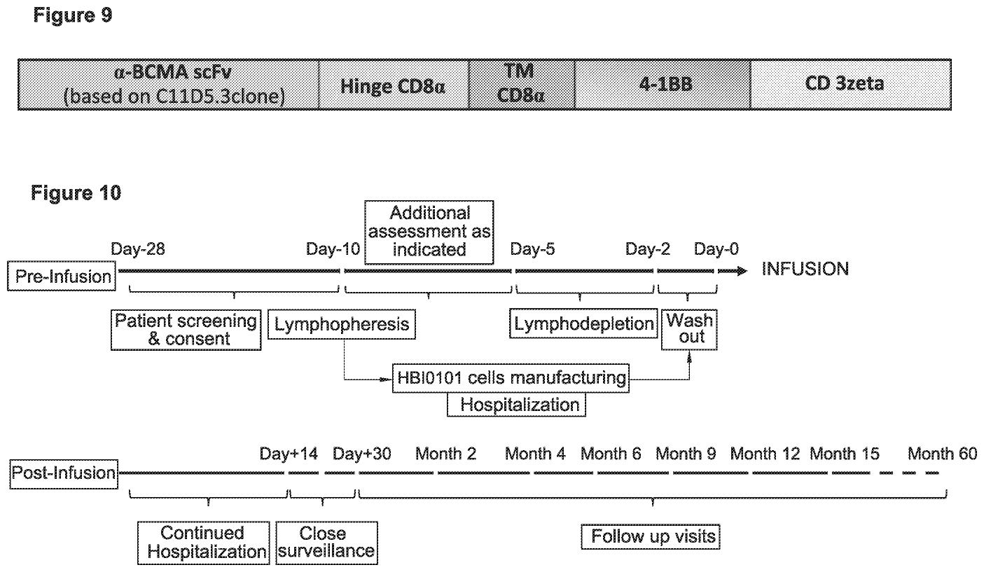

### FIG. 9. Schematic of HBI0101 CAR Construct

C11D5-3-CD8-41-BBZ encoding the anti-BCMA CAR is depicted. BCMA CAR sequence consists of a C11D5-3 anti-BCMA single chain variable fragment (scFv), CD8α hinge and transmembrane regions, the cytoplasmic portion of the 4-1BB costimulatory molecule, and the CD3ζ T-cell activation domain.

### FIG. 10. HBI0101-Clinical Study Outline

Before enrolment, candidates are screened for their eligibility. At day −10 prior to infusion, lymphocytes are collected using the Sprectra Optia apheresis instrument. Patients are then hospitalized for baseline assessment, and HBI0101 production from the fresh/frozen apheresis is initiated. On days −5 to −2, patients are T-cells depleted with fludarabine and cyclophosphamide, and after two days of “wash-out” from lymphodepletion, infused with the manufactured HBI0101 (day 0). After infusion, patients are hospitalized for two weeks for safety follow up. Routine follow-up at the indicated time points (1, 2, 4, 6, 9, 12, 15 . . . 60 months after HBI0101 infusion) is performed for five years or until patient's discontinuation from the study.

### FIG. 11. The “3+3” Dose Escalation Study Design

DLT, dose limiting toxicity; MTD, maximum tolerable dose.

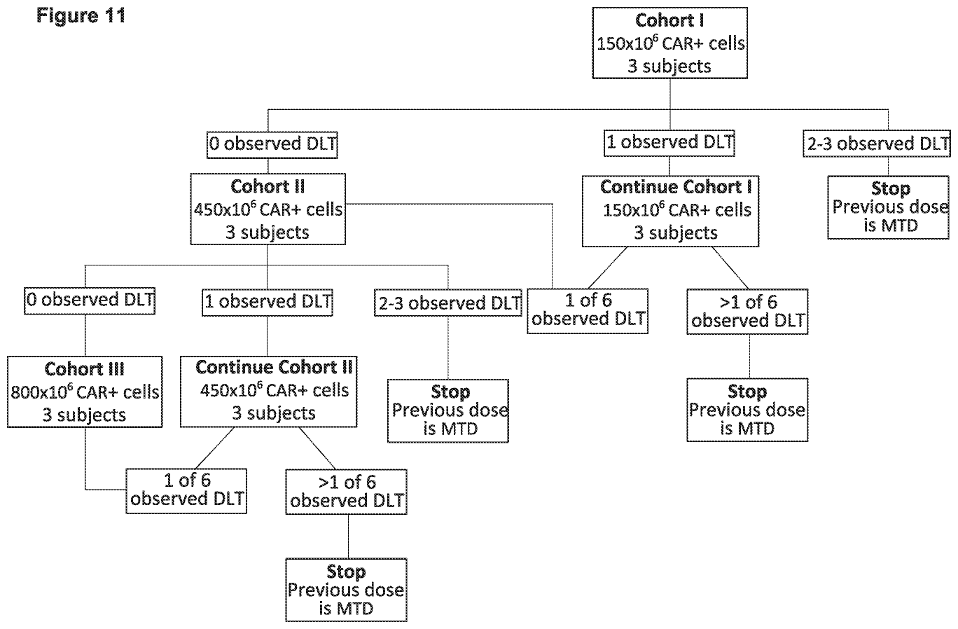

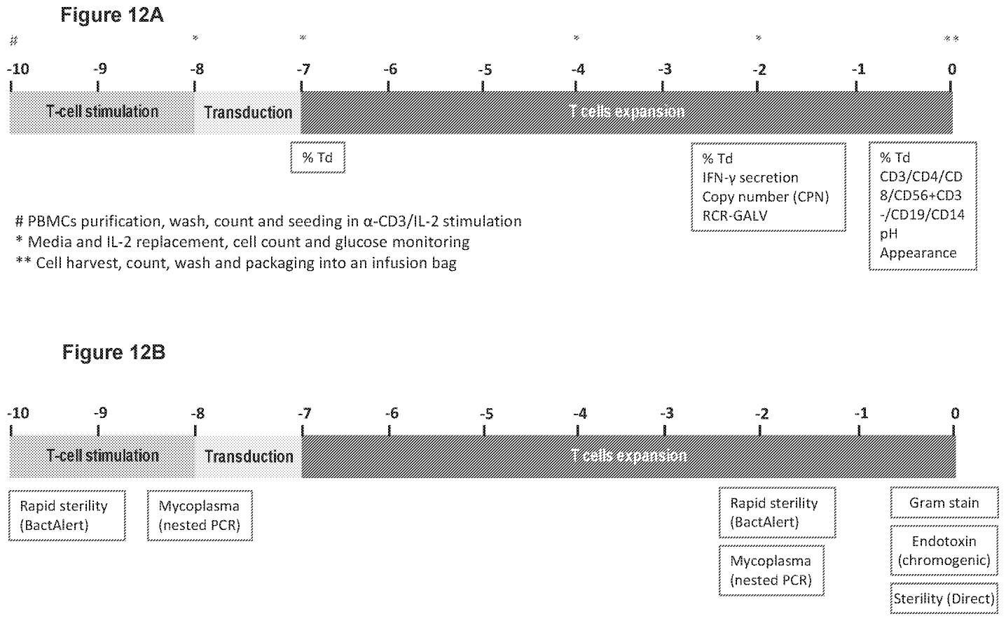

### FIG. 12A-12B. QC and Sterility Testing Along HBI0101 Manufacture

FIG. 12A. HBI0101 culture samples were collected at the indicated time points for QC. FIG. 12B. HBI0101 culture samples were collected at the indicated time points for sterility in-process (IE) and end-of-process (EOP) control. Td, transduction, CPN, copy number.

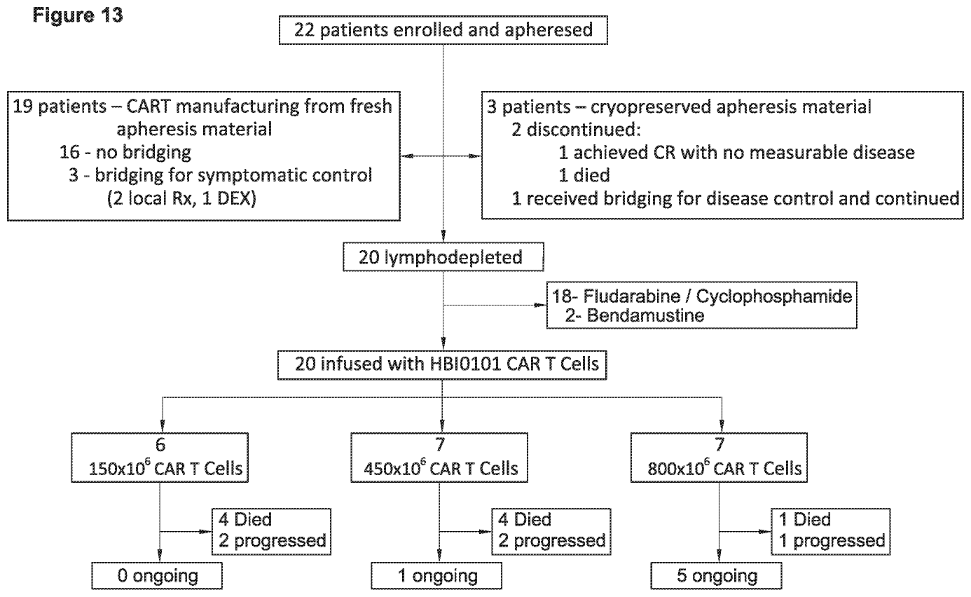

### FIG. 13. Consort Diagram

A total of twenty MM patients, out of the 22 patients that were enrolled and leukapheresed, underwent lymphodepletion, and were treated according to a “3+3” dose escalation study design: cohort 1 was administered with 150×10\hat{ }6 CAR+ cells (N=6), cohort 2 with 450×10\hat{ }6 CAR+(N=7) and cohort 3 with 800×10\hat{ }6 CAR+(N=7). DEX, dexamethasone; Rx, radiation.

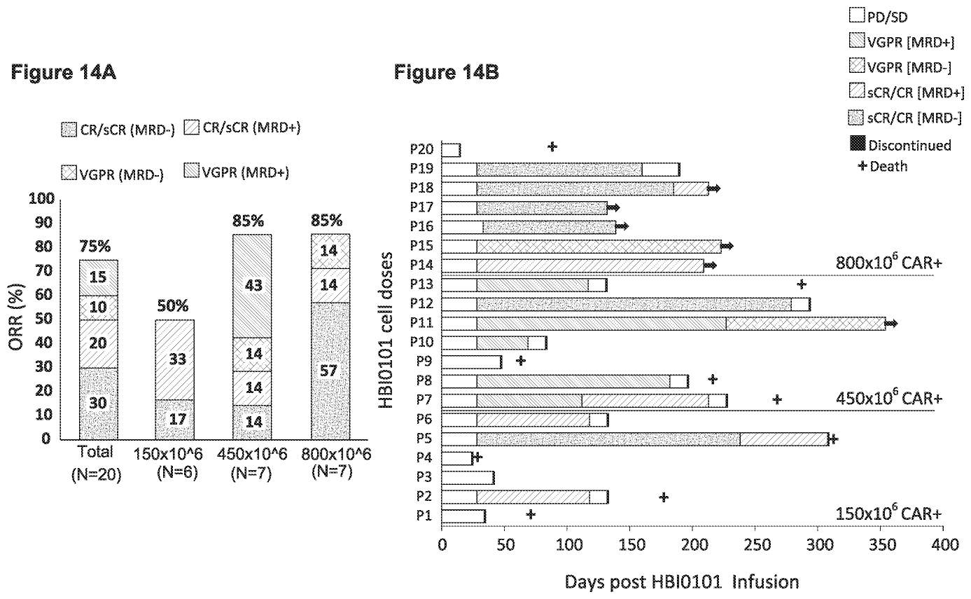

FIG. 14A-14B. Objective Responses Inpatients Treated with HBI0101 CART Cells

FIG. 14A. Overall response (OR) rate. The best responses for each patient are shown, grouped by dose cohorts (150-, 450- and 800×10⁶ CAR+ cells/dose). Disease response was determined according to the International Myeloma Working Group (IMWG) consensus criteria [Kumar S, et al. Lancet Oncol. 17(8):e328-e346 (2016)]. Minimal residual disease (MRD) is defined as the number of MM plasma cells detected in the bone marrow per 1×10⁵ total nucleated cells. An MRD of 1×10\hat{ }-5 or less is considered MRD-negative.

FIG. 14B. Response to HBI0101-treatment. Swimmer's plot of best responses among individual MM patient are shown according to cell dose (150- to 800×10\hat{ }6 CAR+). Response assessment according to IMWG criteria. Grey—progressive disease (PD); light grey—stable disease (SD); green—very good partial response (VGPR); light blue—stringent complete response/complete response with MRD positive (sCR/CR (MRD+)); blue—stringent complete response/complete response with MRD negative (sCR/CR (MRD−)).

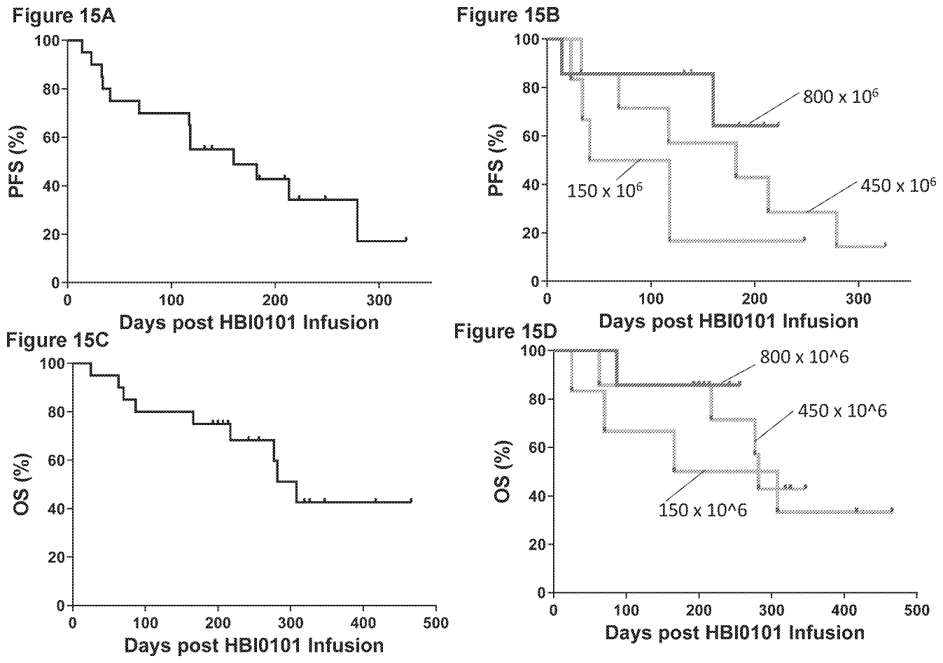

FIG. 15A-15D. Survival of Patients with R/R MM Administered with HBI0101 CAR T Cell

FIG. 15A. Kaplan-Meier analysis of progression-free survival (PFS) in all the patients.

FIG. 15B. Kaplan-Meier analysis of progression-free survival (PFS) grouped by dose cohorts.

FIG. 15C. Kaplan-Meier analysis of overall survival (OS) in all the patients overall.

FIG. 15D. Kaplan-Meier analysis of overall survival (OS) grouped by dose cohorts.

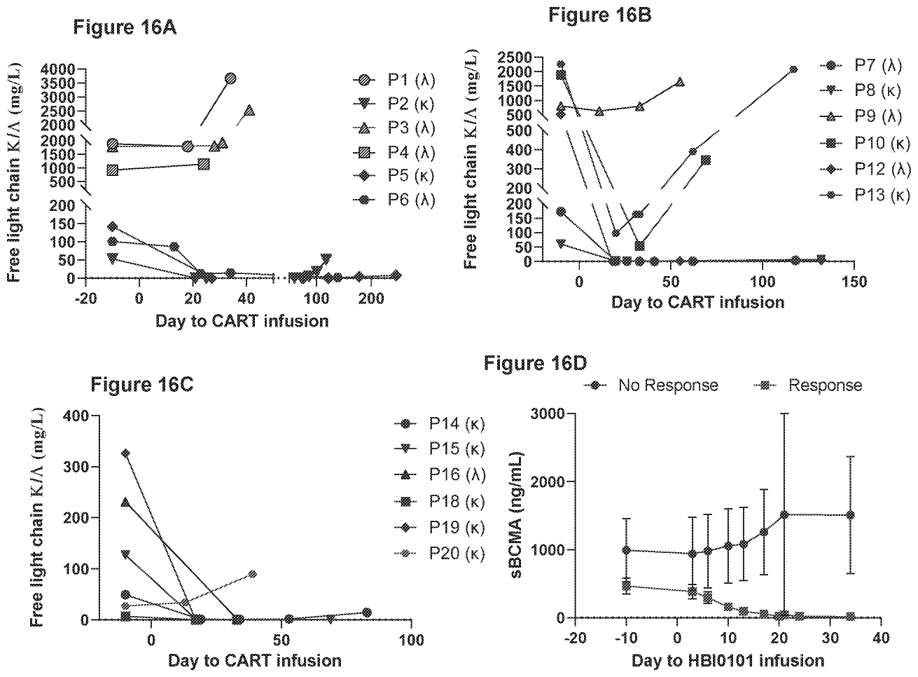

### FIG. 16A-16D. MM Disease Monitoring

FIG. 16A-16C. Free light chain levels were determined at the indicated time points prior to and following HBI0101-infusion in cohort 1 (N=6) (FIG. 16A), cohort 2 (N=6) (FIG. 16B) and cohort 3 (N=6) (FIG. 16C). Normal range for Kappa light chain: 6.7-22.4 mg/L, and Lambda light chain: 8.3-27 mg/L.

FIG. 16D. Soluble BCMA (sBCMA) levels prior to and following CART infusion were determined by ELISA in the “response” (blue squares) vs. “no response” (black dots) group.

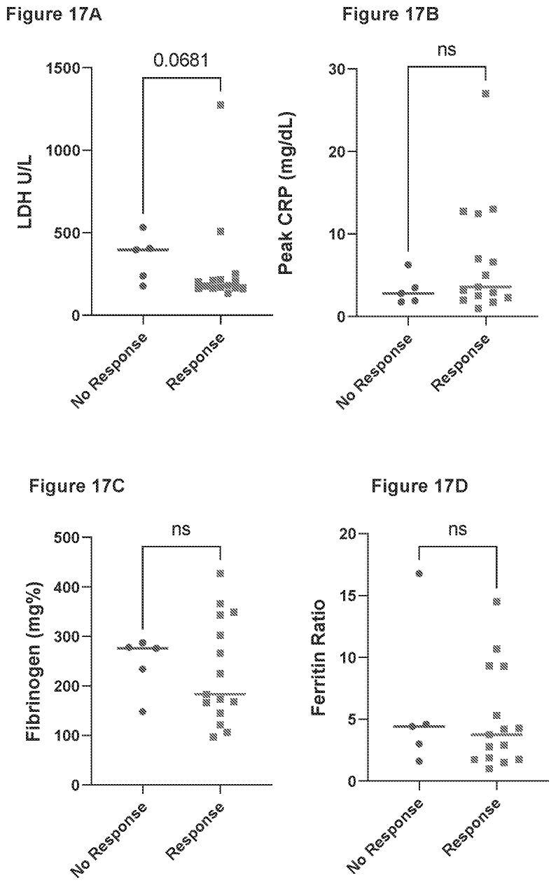

### FIG. 17A-17D. Correlation Between Laboratory Parameters and Response to HBI0101

FIG. 17A. shows Lactate dehydrogenase (LDH) levels assessed at day 14 post HBI0101 infusion.

FIG. 17B. shows Peak C-reactive protein (CRP) levels assessed at day 14 post HBI0101 infusion.

FIG. 17C. shows Fibrinogen levels assessed at day 14 post HBI0101 infusion.

FIG. 17D. shows Ferritin ratio assessed at day 14 post HBI0101 infusion.

Bars represent the median.

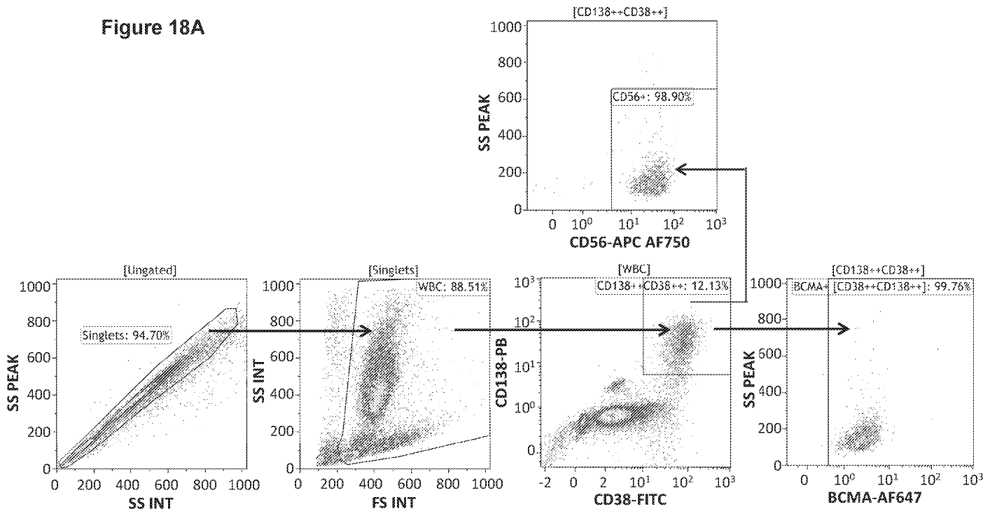

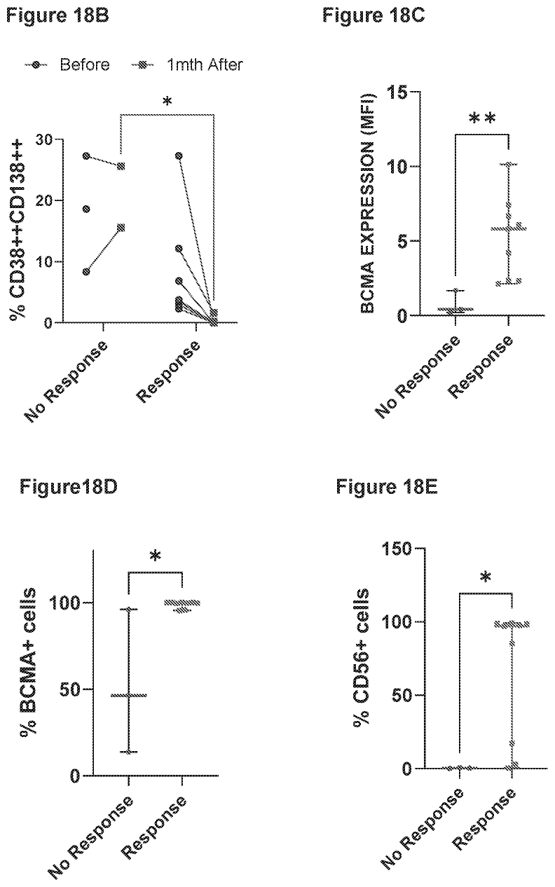

### FIG. 18A-18E. Efficacy of HBI0101 CART-Based Therapy in Eliminating CD38++CD138++ Malignant PCs

FIG. 18A. Flow cytometric gating strategy for the assessment of BCMA and CD56 expression on MM-PCs is illustrated by arrows.

FIG. 18B. BM samples prior to and one month following HBI0101-CART infusion were assessed for the presence of PCs (as % of CD38++CD138++ cells) by flow cytometry.

by gating on white blood cells (WBC), as illustrated in (FIG. 18A).

FIG. 18C-18E. Analysis of BCMA and CD56 expression on MM-PCs, by “response” (blue squares) vs. “no response” (black dots) group.

FIG. 18C-18D. BCMA expression levels in multiple myeloma patients (N=12). The mean fluorescence intensity (MFI) (FIG. 18C) and the percent of BCMA positive PCs (FIG. 18D) were determined by flow cytometry.

FIG. 18E. Percent of CD56-positive PCs in “response” (N=9) vs. “no response” (N=3) group. Samples were gated on CD38++CD138++ cells (as illustrated in (FIG. 18A)).

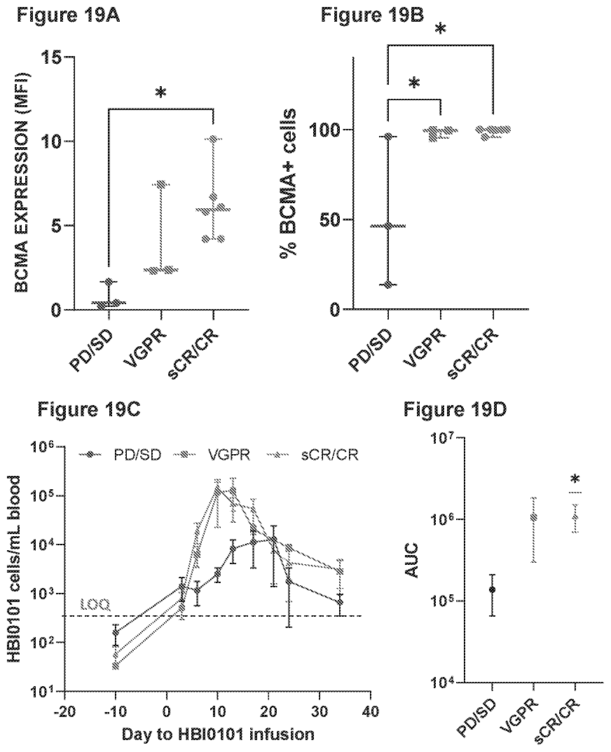

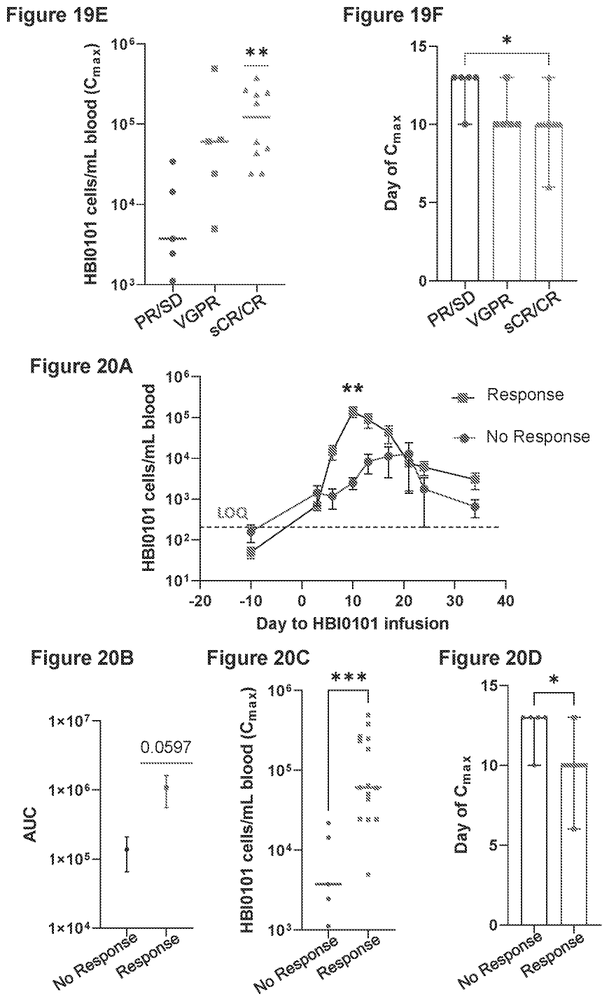

### FIG. 19A-19F. Analysis of BCMA Expression on MM-PCs; HBI0101 CART In Vivo Kinetics

FIG. 19A-19B. Analysis of BCMA expression on MM-PCs. HBI0101-treated MM patients were classified according to SD/PD (black dots, N=3)), VGPR (green squares, N=3)) and sCR/CR (blue circles, N=6) groups. The mean fluorescence intensity (MFI) (FIG. 19A) and the percent of BCMA-positive PCs (FIG. 19B) were determined by flow cytometry. (FIG. 19C-FIG. 19F) HBI0101 CART in vivo kinetics in MM patients treated with HBI0101 and classified by SD/PD (dots, N=5), VGPR (squares, N=5) and sCR/CR (circles, N=10).

FIG. 19C. The median number of HBI0101-CART cells per 1 mL blood in the SD/PD vs. VGPR and sCR/CR groups was determined by quantification of CAR transgene levels by qRT-PCR method following CART infusion at the indicated times and further adjusted to the copy numbers per transduced cell at the day of CART infusion. The limit of quantitation (LOQ) was 500 CART/mL blood.

FIG. 19D. HBI0101 CART cell overall expansion in the first month of CART therapy. Area under the curve (AUC) as a measure of CART overall expansion was calculated with Prism software (GraphPad).

FIG. 19E. HBI0101 cells in-vivo median concentration at peak (Cmax) in the SD/PD (dots) vs. VGPR (squares) and sCR/CR (triangles) groups.

FIG. 19F. Median time to Cmax (Tmax) in the SD/PD vs. VGPR and sCR/CR groups. Upper and lower bars I represent the maximal and minimal values, respectively. SD, stable disease; PD, progressive disease; VGPR, very good partial response; (s)CR, (stringent) complete response. * p<0.05, **p<0.01, by unpaired t-test.

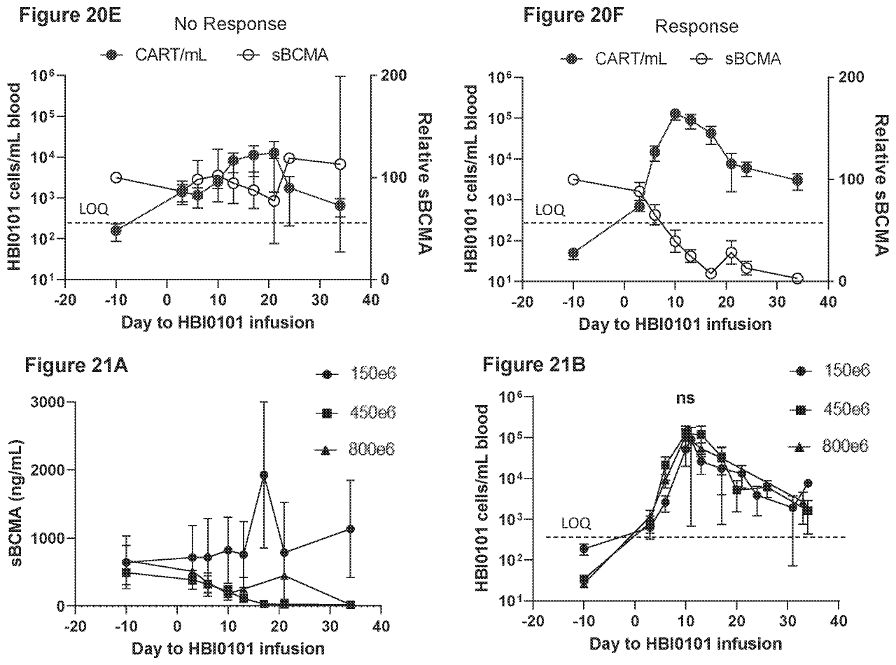

FIG. 20A-20F. sBCMA Clearance and HBI0101 CART In Vivo Kinetics

FIG. 20A. The median number of HBI0101-CART cells per 1 mL blood in the “response” vs. “no response” group was determined by quantification of CAR transgene levels by qRT-PCR method following CART infusion at the indicated times and further adjusted to the copy numbers per transduced cell at the day of CART infusion. The limit of quantitation (LOQ) was 500 CART/mL blood.

FIG. 20B. HBI0101 CART cell overall expansion in the first month of CART therapy. Area under the curve (AUC) as a measure of CART overall expansion was calculated with Prism software (GraphPad).

FIG. 20C. HBI0101 cells in vivo median concentration at peak (Cmax) in “response” (squares) vs. “no response” (dots) group.

FIG. 20D. Median time to Cmax (Tmax) in “response” (squares) vs. “no response” (dots) group. Upper and lower bars represent the maximal and minimal values, respectively.

FIG. 20E-20F. Soluble BCMA (sBCMA) levels prior to and following HBI0101 infusion determined by ELISA and further normalized to sBCMA concentration at baseline (right y-axis; empty circles), vs. HBI0101-cell expansion indicated by the CART/mL (left y-axis; filled circles) in the “no response” group (FIG. 20E), and in the “response” group (FIG. 20F). **p<0.01, by unpaired t-test.

FIG. 21A-21B. Analysis of HBI0101 Cells Persistence and sBCMA Levels by Dose Cohort

FIG. 21A. Soluble BCMA (sBCMA) levels prior to and following CART infusion determined by ELISA in the different dose-cohorts. Legend as follows: black circles, 150×10⁶ CAR+; black squares, 450×10⁶ CAR+ and black triangles, 800×10⁶ CAR+ cells. Statistical analysis by 2-way ANOVA.

FIG. 21B. HBI0101 in-vivo kinetics according to HBI0101 dose. The number of HBI0101 CART per 1 mL blood was determined by quantification of CAR transgene levels by qRT-PCR method following CART infusion at the indicated times, and further adjusted to the CPN of HBI0101 cells before infusion. The limit of quantitation (LOQ) was 500 CART/mL blood.

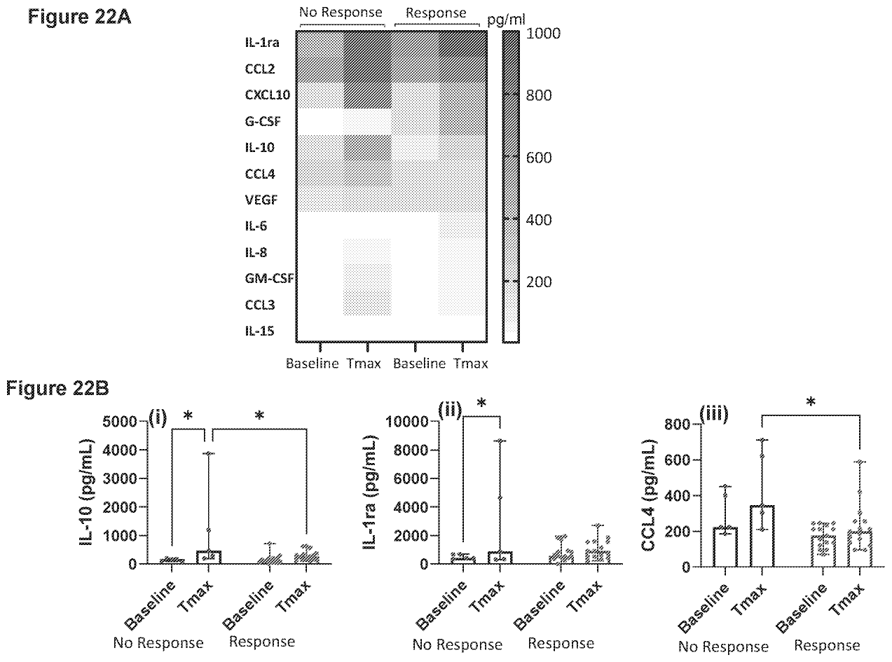

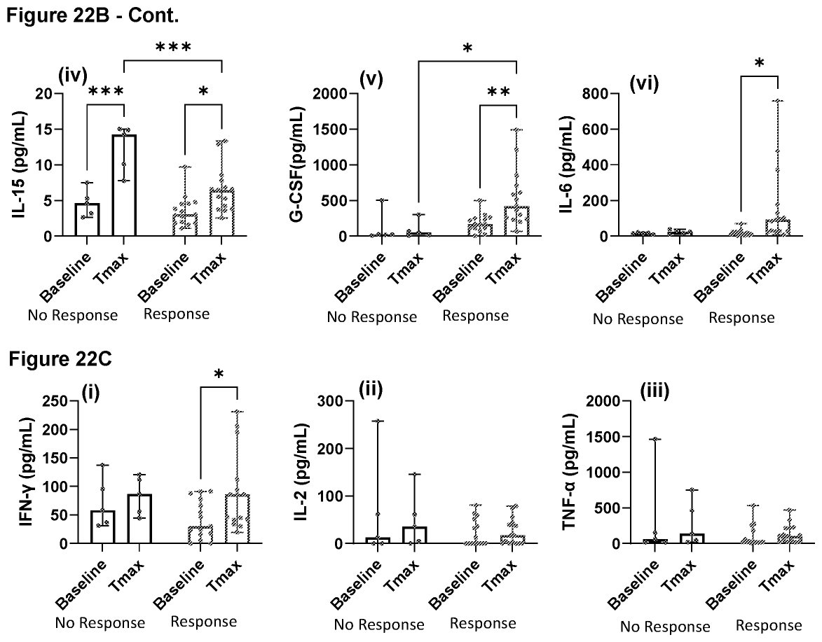

### FIG. 22A-22C. Comparison of Cytokine Profile for Predicting Response to HBI0101-Treatment

FIG. 22A. Heat map analysis of 12 cytokine detected in serum samples of “response” patients (N=15) vs. “no response” (N=5) patients, at baseline (at day −10) and at Tmax following HBI0101-treatment. Rows represent the cytokines that were affected by HBI0101-treatment in the, while columns represent the median of the cytokine values of the patients of the “response” and “no response” groups, “before” and “after” HBI0101-infusion. Cytokines values at day of Cmax following CART infusion in the “response” group were used as reference values. Key color legend is indicated, with intense dark colors for high cytokine values, and light color for low cytokine values. Median and range values of the represented cytokines are detailed in Table 6.

FIG. 22B(i)-22B(vi)-22C(i)-22C(iii). Graphical representation of the cytokines that were significantly differentially expressed between the “response” and “no response” groups, prior to and following HBI0101 infusion. More specifically, for FIG. 22B: IL-10 (22B(i)), IL-1ra (22B(ii)), CCL4 (22B(iii)), IL-15 (22B(iv)), G-CSF (22B(v)), and IL-6 (22B(vi)), for FIG. 22C: IFN-γ (22C(i)), IL-2 (22C(ii)) and TNF-α (22C(iii)). Black lines represent the median, upper and lower bars I represent the maximal and minimal values, respectively. R, response; NR, no response. Statistical analysis by 2-way ANOVA.

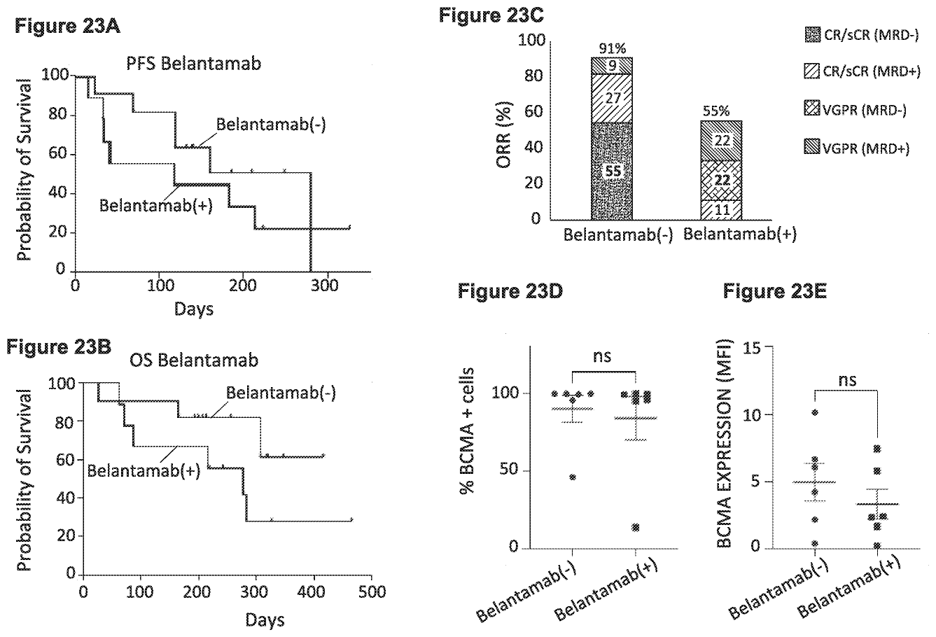

### FIG. 23A-23D. Effect of Belantamab Pre-Treatment on MM Patients' Response to HBI0101 Therapy

FIG. 23A-23B. Kaplan-Meier analysis of progression-free survival (PFS) (FIG. 23A) and overall survival (OS) (B) in “Belantamab(+)” vs. “Belantamab(−)” group.

FIG. 23C-23D. Effect of belantamab prior therapy on PCs BCMA expression. Percent of BCMA-expressing BM-PCs (FIG. 23C) and BCMA expression MFI (FIG. 23D) on BM-PCs were determined by flow cytometry prior to HBI0101 infusion and analyzed according to patient's preexposure to belantamab. Samples were gated on CD38++CD138++ cells.

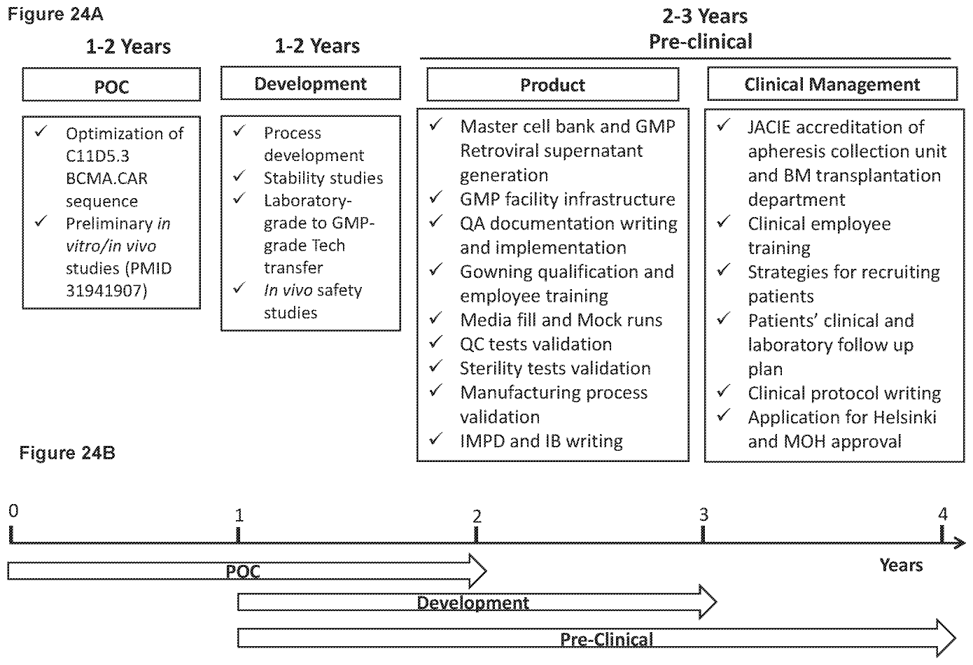

### FIG. 24A-24B. Main Steps in the Bench-to-Bedside Translation of HBI0101 CART-Based Therapy

FIG. 24A. POC, proof-of-concept; GMP, Good Manufacturing Practice; IMPD, Investigational Medicinal Product Dossier; IB, Inventor's Brochure; JACIE, The Joint Accreditation Committee ISCT-Europe & EBMT; MOH, Ministry of Health, QA, quality assurance; QC, quality control.

FIG. 24B. shows time-line for the different stages.

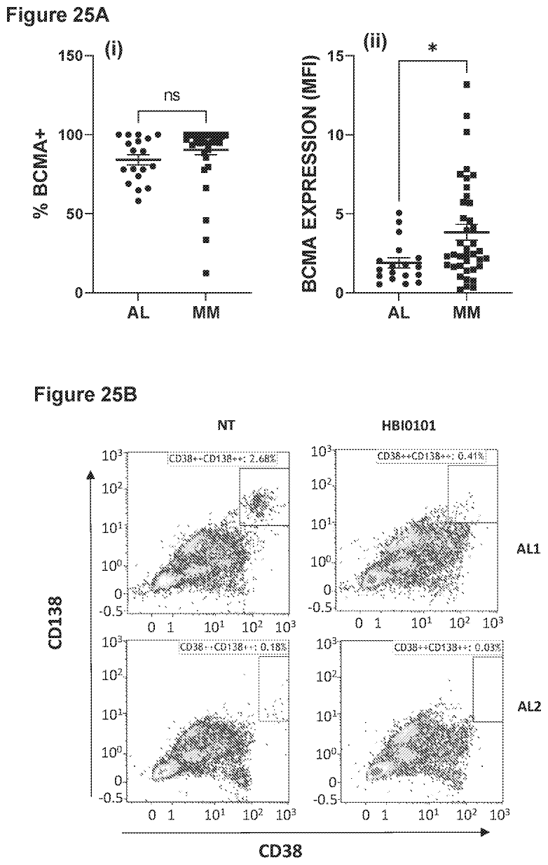

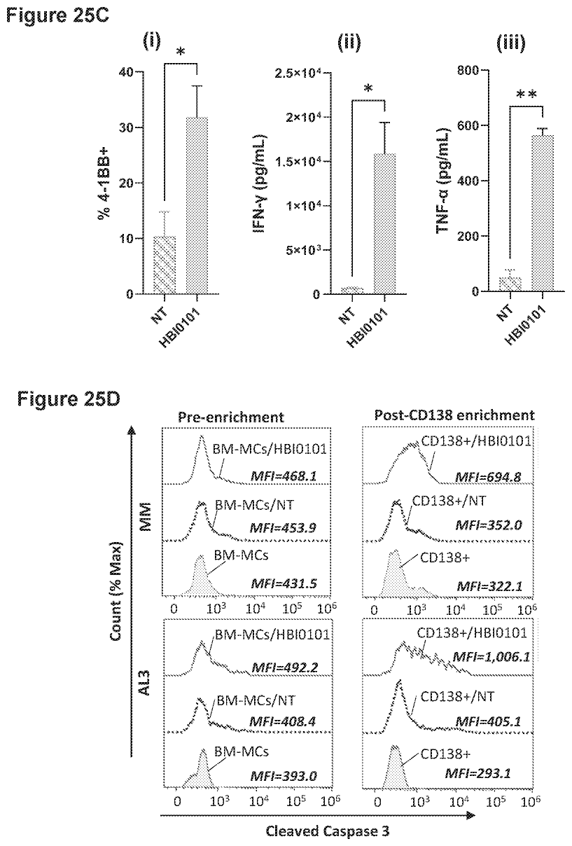

### FIG. 25A-25D. HBI0101 Preclinical Evaluation

FIG. 25A(i)-25A(ii). BCMA expression levels on amyloidosis- and multiple myeloma-PCs PCs and preclinical evaluation. BCMA expression levels in patients with AL (n=18) and multiple myeloma (n=39). BCMA % of expression (FIG. 25A(i)) and MFI (FIG. 25A(ii)) were determined by flow cytometry. Samples were gated on CD38++CD138++ cells.

FIG. 25B. Elimination of bone-marrow (BM)-derived primary plasma cells (PCs) (gated on CD38++CD138++) of AL donors following overnight co-incubation (1:1 E:T ratio) with autologous HBI0101 CART cells, in comparison with non-transduced (NT) cells.

FIG. 25C(i)-(iii). HBI0101 CAR T activation following overnight co-incubation of BM-derived primary plasma cells (PCs) of AL1 and AL2, as indicated by the percent of CD137+(4-1BB+) cells (gated on CAR+ cells). Co-culture with autologous NT cells serves as control. n=2, p<0.05 (FIG. 25C(i)). FIGS. 25C(ii)-(iii): Cytokines secretion by HBI0101 CART or NT cells following co-incubating with BM-MNCs (P<0.05).

FIG. 25D. Increased apoptosis of MM- and AL3-CD138+ magnetically enriched cells following co-incubation with autologous HBI0101 CAR T cells for two hours, comparing with autologous NT control cells. Co-cultures of CD138− cell fraction with autologous CAR T or NT cells, serve as control. Apoptosis was assessed by determination of the intracellular level of cleaved caspase-3 in CD3-BCMA+ plasma cells by flow cytometry.

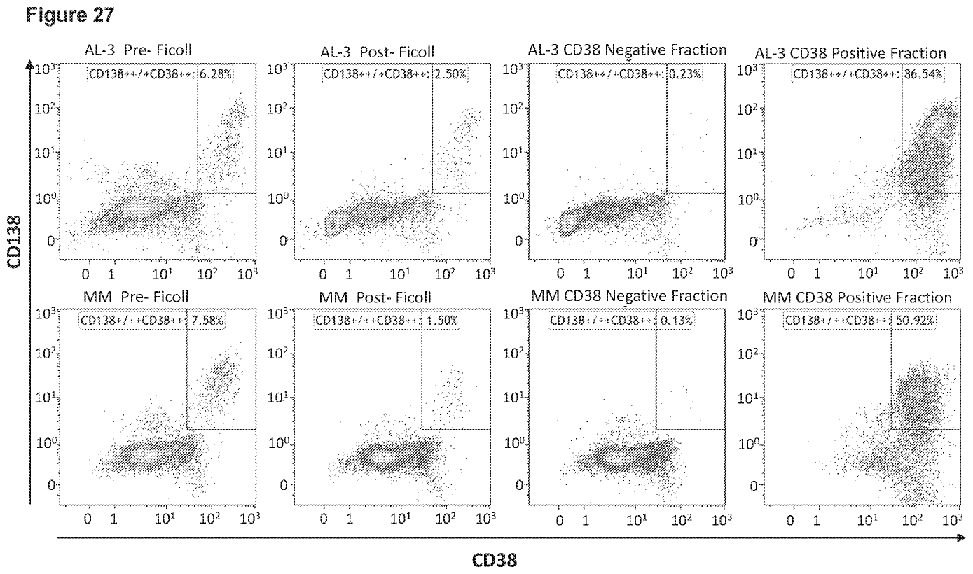

Of note, the percent of CD138+ cells prior to magnetic isolation were 1.50% in the patient with multiple myeloma and 2.50% in the patient with AL, and significantly increased following enrichment (50.9% and 86.5%, respectively; see FIG. 27.

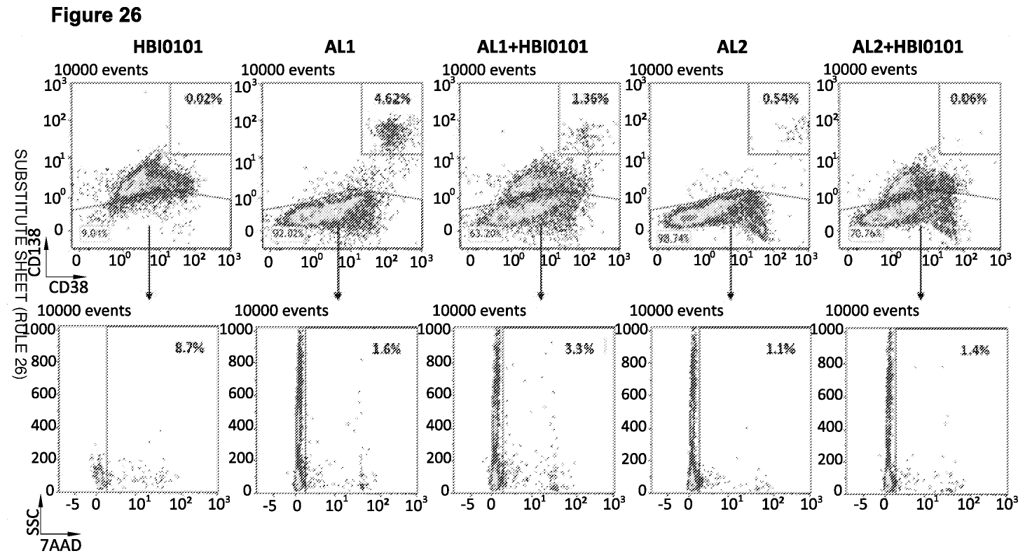

### FIG. 26. Non-Plasma Cell Viability

BM-MNCs derived from AL1 and AL2 were co-culture with HBI0101 or non-transduced (NT) cells of AL1 (autologous vs. allogeneic settings) for 1 hour. Non-plasma BM-MNCs viability was assessed by flow cytometry using 7-AAD staining (bottom panel). Samples were analyzed by gate out of CD38++CD138++ and CART cells, as indicated in the upper panel.

FIG. 27. Plasma Cells (PCs) Enrichment from AL- and MM-Bone Marrow Aspirate

Bone marrow-mononuclear cells (BM-MNCs) were isolated from fresh bone marrow samples of AL-Patient 3 (upper panels) and MM-patient (bottom panels) after Ficoll fractionation. CD138+ PCs were magnetically sorted using the EasySep™ Human CD138 Positive Selection Kit II. The percent of PCs prior and post CD138+ enrichment, in the negative and positive fractions, was determined by flow cytometry (samples were gated on CD38++ CD138++ cells).

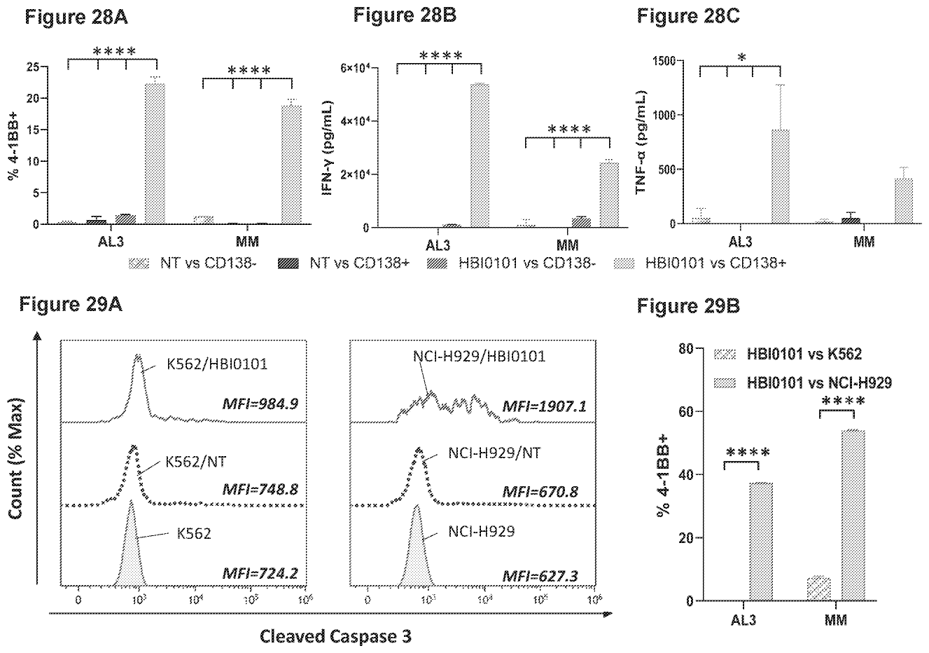

### FIG. 28A-28C. HBI0101 Preclinical Evaluation

FIG. 28A. 4-1BB upregulation in HBI0101 CART or NT cells following co-incubating with autologous CD138+ or CD138− cell fraction (gated on CAR+ cells). HBI0101 vs. NT, p<0.0001; HBI0101:CD138+vs. HBI0101:CD138−, p<0.0001.

FIG. 28B-28C. Cytokines secretion by HBI0101 CART or NT cells following co-incubating with autologous CD138+ or CD138− cell fraction (IFN-γ, TNF-α, respectively).

### FIG. 29A-29B. Specificity of BCMA.CART (HBI0101) Cells on BCMA-Expressing Cells Targeting

FIG. 29A. Increased apoptosis of BCMA-expressing NCI-H929 myeloma cells but not of the BCMA-negative K562 chronic myelogenous leukemia (CML) cells following 2 hours co-incubation with HBI0101 cells. Target cells were not affected by the presence of non-transduced (NT) cells, regardless of BCMA expression. Apoptosis is indicated as the percent of cleaved caspase-3 cells (gated on H929 or K562 target cells).

FIG. 29B. Upregulation of the 4-1BB activation marker at the surface of HBI0101 cells following an overnight incubation with BCMA-expressing H929 myeloma cells, but not following incubation with BCMA-negative K562 cells (p<0.0005). 4-1BB expression was barely detected at the surface of NT cells, regardless of whether they were in co-culture with H929 or K562 target cells. The percent of 4-1BB+ cells was determined by flow cytometry (samples were gated on CAR+ cells).

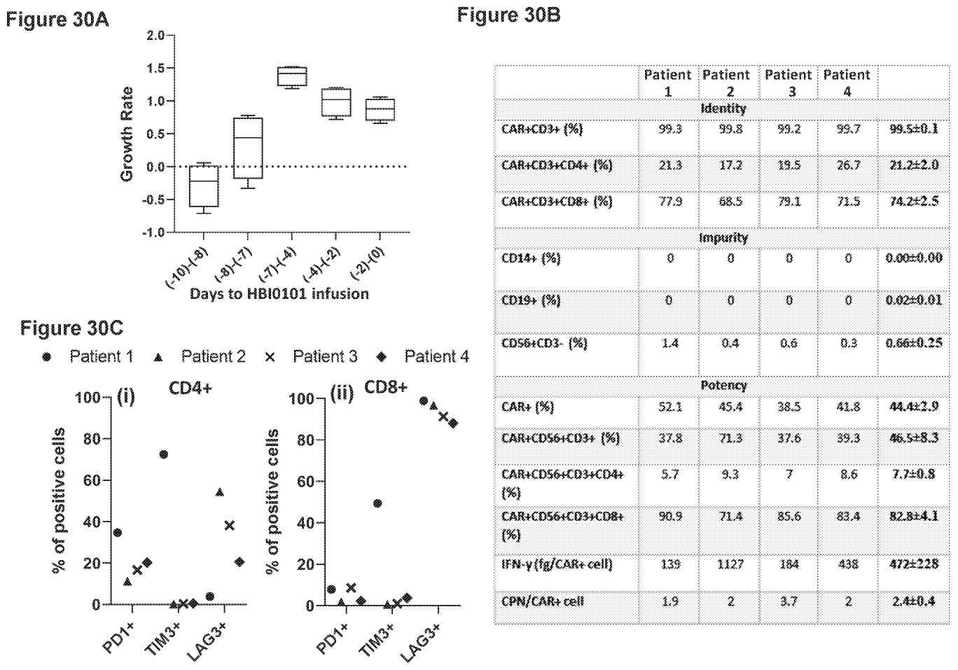

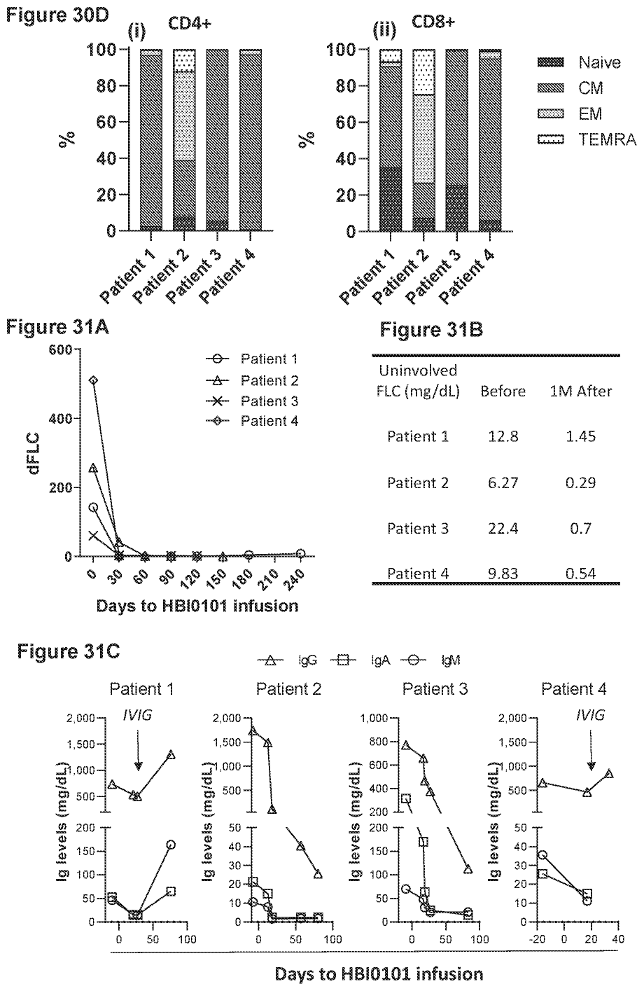

FIG. 30A-30D. Feasibility of HBI0101 CART Production from AL Amyloidosis Patients

FIG. 30A. Growth rate of HBI0101-transduced cells of AL patients along CART production process. Drug products (DPs) were generated from four AL patients' leukaphereses. The production process lasts 10 days for all four patients. The peak in cell growth was observed between days −7 to −4.

FIG. 30B. Characterization of HBI0101 drug products (DPs) at day of infusion (day 0). DP Identity. The percent of CD3+, CD4+ and CD8+ cells (gated on live CAR+ cells) was determined by flow cytometry. DP Impurity. The percent of “cell impurities”, as to CD19+, CD14+ and CD3-CD56+(gated on live cells) was determined by flow cytometry, and do not exceed 1% each in all four batches. The percent of BCMA.CAR+ cells (gated on live cells) was determined by flow cytometry using human recombinant BCMA protein. DP potency. The copy number (CPN) of HBI0101 inserts into every transduced cell was quantified by qRT-PCR and further adjusted to transduced cell as described in the supplementary material and method section. Potency of the drug product. The percent of activated cytotoxic T cells (CD3+CD56+) was determined by flow cytometry (gated either on CD4+ or CD8+ T cells). The secretion of IFN-γ by each HBI0101 transduced cell, was assessed by ELISA at day −2. IFN-γ was released into the co-culture supernatant of HBI0101-transduced cells with NCI-H929 (1:1 E:T ratio) following an overnight incubation. The total amount of IFN-γ in each culture well was quantified and then divided by the number of transduced cells introduced into the well.

FIG. 30C(i)-30C(ii). Exhaustion profile of the DPs at the day of infusion (day 0) was established by assessing the percent of PD-1+, LAG-3+ and TIM-3+ cells by flow cytometry (gated on CAR+ and CD4+[FIG. 30C(i)] or CD8+[FIG. 30C(ii)]T cells).

FIG. 30D(i)-30D(ii). Differentiation profile of DPs at the day of infusion was established by assessing the percent of CD45RA+/CCR7+ (Naive cells), CD45RA−/CCR7+(Central Memory-CM), CD45RA−/CCR7− (Effector Memory-EM) and CD45RA+/CCR7− (Terminally differentiated Effector Memory-TEMRA) by flow cytometry (gated on CAR+ cells and on CD4+[FIG. 30D(i)] or CD8+[FIG. 30D(ii)]T cells).

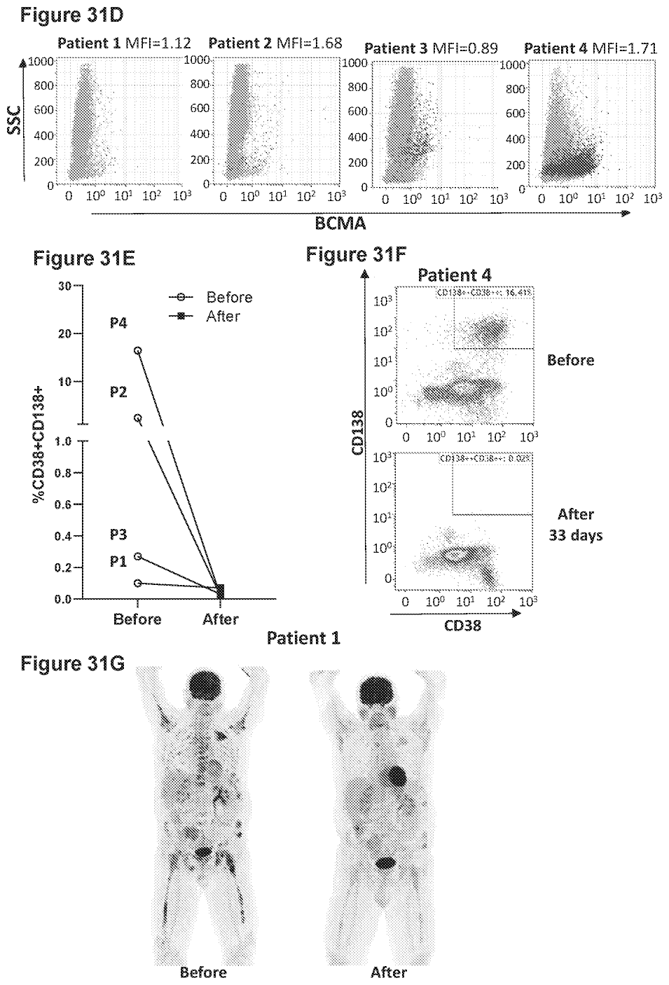

### FIG. 31A-31G. Clinical Outcome of HBI0101 CART-Based Therapy in the Treatment of AL Amyloidosis

FIG. 31A. Difference between involved (iFLC) and uninvolved FLC (dFLC) of 4 AL patients was assessed at baseline and post HBI0101 CART infusion.

FIG. 31B. Uninvolved FLC of 4 patients with AL was assessed at baseline and post HBI0101 CART infusion at day 30. Normal range for Kappa light chain: 6.7-22.4 mg/L, and Lambda light chain: 8.3-27 mg/L.

FIG. 31C. Immunoglobulins levels prior to and following HBI0101 CART infusion were monitored over time. The mark #mentions intravenous Ig (IVIG) intake by Patients 1 and 4 at day 26 post CART infusion.

FIG. 31D. BCMA median expression (mean fluorescence intensity; MFI) in AL patients' bone-marrow-derived plasma cells (gated on CD38++CD138++ cells) prior to HBI0101 CART infusion was assessed by flow cytometry. Red dots represent PCs (gated on CD38++CD138++ cells), while grey dots represent non-PCs.

FIG. 31E. Demonstrating efficacy of HBI0101 CART-based therapy in eliminating CD38++CD138++ AL primary plasma cells. Bone-marrow samples prior to and one month following HBI0101 CART infusion were assessed for the presence of plasma cells by flow cytometry (by gating on CD38++CD138++ cells).

FIG. 31F. Dot plot representation of Patient 4's flow cytometry data depicted in (FIG. 31E).

FIG. 31G. Positron emission tomography-computed tomography (PET-CT) scans from before and after HBI0101 CART treatment.

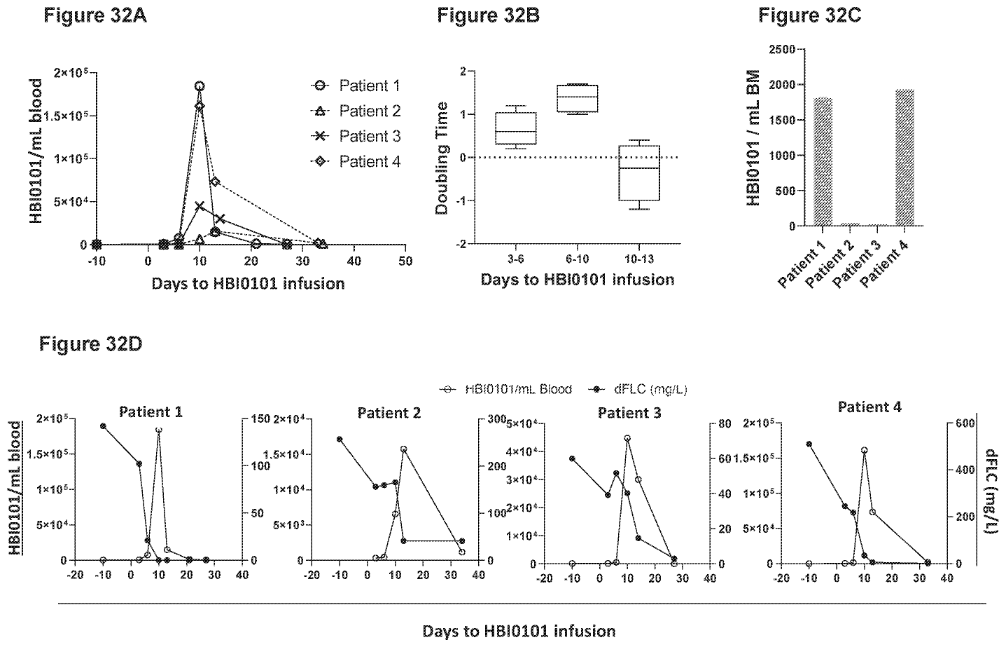

### FIG. 32A-32D. HBI0101 In Vivo Kinetic

FIG. 32A. The number of HBI0101 CART per 1 mL blood was determined by quantification of CAR transgene levels by qRT-PCR method following CART infusion at the indicated times, and further adjusted to the percent of transduction at the day of CART infusion. The limit of quantitation (LOQ) 10² CART/mL blood.

FIG. 32B. HBI0101 cells in vivo expansion rates. Black lines represent the median, upper and lower bars I represent the maximal and minimal values, respectively.

FIG. 32C. HBI0101 CART/mL in AL patients' bone marrow fluid, 1 month after CART infusion (determined as detailed in (FIG. 32A)).

FIG. 32D. Difference between involved and uninvolved FLC (dFLC) levels prior to and following CART infusion (right y-axis; filled circles) is compared with CART cell expansion indicated by the CART/mL (left y-axis; empty circles).

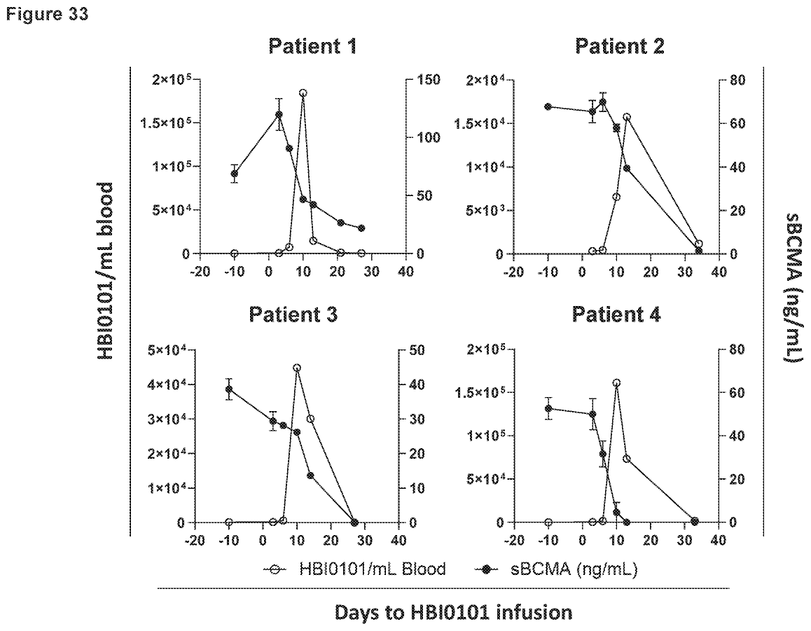

FIG. 33. HBI0101 Versus sBCMA In-Vivo Kinetics

Serum BCMA (sBCMA) levels prior to and following CART infusion determined by ELISA (right y-axis; filled circles) is described in comparison with CART cell expansion in blood (left y-axis; empty circles).

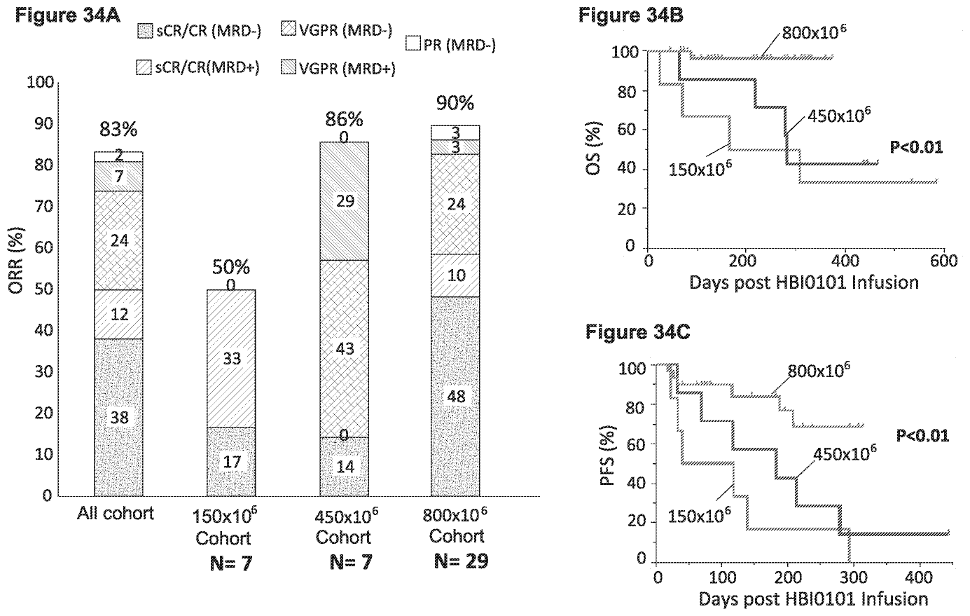

FIG. 34A-34C. Efficacy Clinical Results with HBI0101 CAR T Cell HBI0101 (N=29 for 800×10⁶ Cohort)

FIG. 34A. Overall response (OR) rate. The best responses for each patient are shown, grouped by dose cohorts (150-, 450- and 800×10⁶ CAR+ cells/dose). Disease response was determined according to the International Myeloma Working Group (IMWG) consensus criteria [Kumar S, et al. Lancet Oncol. 17(8):e328-e346 (2016)]. Minimal residual disease (MRD) is defined as the number of MM plasma cells detected in the bone marrow per 1×10⁵ total nucleated cells. An MRD of 1×10⁻⁵ or less is considered MRD-negative.

FIG. 34B. Kaplan-Meier analysis of progression-free survival (OS) grouped by dose cohorts.

FIG. 34C. Kaplan-Meier analysis of overall survival (PFS) grouped by dose cohorts.

### EXAMPLES

### Experimental Procedures

### PBMCs and Cell Lines

Peripheral blood mononuclear cells (PBMCs) were from healthy donors obtained from the Israeli Blood Bank (Sheba Medical Center, Tel-Hashomer, Israel, from the Pheresis Collection Unit, (IRB approval No. 0458-19-HMO) or from blood of patients with plasma cell malignancies. Blood was collected from patients with plasma cells malignancies in accordance with the IRB approval No. 0253-20-HMO.

Bone marrow (BM) aspirates from patients with plasma cell dyscrasias were obtained in accordance with Helsinki approval from the Ethical Committee of Hadassah Ein-Kerem Medical Center. BM-derived mononuclear cells were isolated by centrifugation over a density gradient medium (lymphocyte separation medium, Lonza).

BCMA⁺ cell lines are RPMI8226 (ATCC/CCL-155), (NCI)-H929 (ATCC/CRL-9068) and MM1.S (ATCC/CRL-2974). K562 (ATCC/CCL-243) is an erythroleukemia line BCMA^{neg}. K562-BCMA was engineered to overexpress BCMA. All cell lines were cultured in RPMI medium (Invitrogen, Carlsbad, CA), with 10% heat-inactivated Fetal Bovine Serum (Biological Industries, Beth Haemek, Israel) and were maintained in a 37° C. and 5% CO2 incubator.

Lymphocytes were cultured in BioTarget medium (Biological Industries, Beth Haemek, Israel) supplemented with 10% heat-inactivated fetal bovine serum (FBS), 1% L-Glutamine, 1% Penicilin/Streptomycin and 300 IU/ml IL-2 (Peprotech Asia, Israel) [Daniel-Meshulam I, et al. Int J Cancer. 13(12):2903-2913. (2013), Eisenberg V, et al. Front Immunol. 8:1212. (2017)., Meril S, et al. Mol Carcinog. 59(7):713-723 (2020)].

### Anti-BCMA Chimeric Receptors

The different chimeras ICBB, IC28 and H8BB (FIG. 1A) were created by overlapping polymerase chain reaction (PCR) based on the heavy chain followed by the light chain derived from the previously described C11D5.3 antibody [Carpenter R O, et al. Clin. Cancer Res. 19(8):2048-2060. (2013)]. ICBB and IC28 incorporated an optimized short IgG4-derived spacer and a Strep Tag [Liu L, et al., Nat. Biotechnol. 34:430-434 (2016)], while H828 and H8BB incorporated CD8α-derived hinge and transmembrane (TM) domains. The H828 CAR was a kind gift from Dr. Kochenderfer, NCI, NIH [Carpenter R O, et al. Clin. Cancer Res. 19(8):2048-2060. (2013)]. These chimeras were subcloned into the well-characterized retroviral vector backbone pMSGV1 (a kind gift from Dr Steven Rosenberg, NCI). Retroviral transduction, co-cultures, ELISAs and cytotoxicity assays were performed as previously described [Hoogi S, et al. J. Immunother. Cancer. 7:243. (2019); Shamalov K, et al., Oncoimmunology. 6:e1285990. (2017). Tal Y, et al. Oncotarget. 5:10949-10958. (2014)].

### Generation of Clinical Grade HBI0101 Retroviral Bank

PG13 HBI0101 were generated by transiently transfecting Phoenix-ECO cells (ATCC) with the plasmid encoding the gamma-retroviral vector MSGV1-HBI0101 using JetPrime reagent (Tamar) and subsequently transducing PG13 cells (ATCC) with HBI0101-Phoenix-ECO cell-free vector supernatants. PG13 transduced population was subsequently sub-cloned by limiting dilution, and the PG1-1-113-10101 expanded to generate a seed bank. The certified PG13 seed bank was sent to the Indiana University Vector Production Facility (IU-VPF) in Indianapolis that has generated a master cell bank (MCB) and a GMP-certified HBI0101 clinical grade retroviral supernatant for the transduction of MM patients' autologous T-cells.

### HBI0101 Cells Production

For ex vivo study: Peripheral blood mononuclear cells from AL and/or multiple myeloma patients' blood were isolated and CD3-activated peripheral blood mononuclear cells were used for further production. Blood was collected from patients with AL or/and multiple myeloma (0253-20-HMO) and processed on a Ficoll gradient (Lymphocyte Separation Medium, Lonza) to isolate peripheral blood mononuclear cells (PBMC). PBMCs were suspended at a concentration of 1×110\hat{ }6 cells per mL in T-cell medium (TCM), containing AIM-V (Gibco) supplemented with 5% human serum (Valley), 1% Glutamax (Gibco). IL2 (300 IU/mL; Proleukin, Novartis) and anti-CD3 monoclonal antibody OKT-3 (50 ng/mL; Miltenyi Biotech) were added to the TCM for 2 days of culture. Tissue culture non-treated 24-well plates were coated with 10 mg/mL RectroNectin (R/N; Takara) in PBS (Lonza) overnight at 4° C., followed by 30 minutes blocking with 2.5% human albumin in PBS, then washed. Retroviral supernatant was thawed, diluted 1:20 with TCM, added to wells, and centrifuged at 2,000 g for 2 hours at 32° C. The supernatant was then aspirated and 0.5×10⁶ CD3-activated PBMCs/mL were seeded into each well in TCM with 300 IU/mL IL2, centrifuged for 10 minutes at 1,000 g, and incubated at 37° C. overnight. Activated but non-transduced (NT) cells were generated and used as T-cell controls. Transduction efficacy was determined at days 6 and 10 of the culture via flow cytometry, by labeling BCMA CAR T cells with the human recombinant BCMA protein (Active; ACRO).

### For Clinical Grade Production:

The production of HBI0101 cells was carried out using the same protocol as described above for the production of the cells for ex vivo applications. Modifications as to the source of the starting material, the use of clinical grade medium and reagents, and production under GMP conditions, were made to generate CART cells suitable for the clinic.

Leucocytes are collected at day −10 by leukapheresis, using the Spectra Optia apheresis system, and then transferred to the Facility for Advanced Cellular Therapy-Hadassah (FACT-H). Cells are separated to peripheral mono-nuclear cells (PBMCS) and T-cell stimulated using anti-CD3 and IL-2 (FIG. 12). At day −8, stimulated T cells are transduced with 1/25 or 1/50 diluted HBI0101 retroviral supernatant overnight. Then, cells are seeded into GRex100 devices filled with AIM-V medium (Gibco) supplemented with 5% human AB serum (Access Cell Culture or Valley), 1% Glutamax (Gibco) and 300 IU/mL IL-2 for seven days of expansion (FIG. 12). Medium and IL-2 replenishment is performed every 2-3 days. At the day of patient's infusion, cells are washed 3 times with saline with 1% human albumin (Kedrion), and then formulated into the final drug product (DP) at the concentration of 15×10⁶ CART cells/mL in saline with 2.5% human albumin. DP infusion volume varied according to cell doses.

### Outline of QC and Sterility Tests for HBI0101 Production

Quality control testing of in-process (IP) and end-of-product (EOP) HBI0101 cells are performed along the manufacturing process as detailed in FIG. 12A, and include: i) determination of the percent of transduction, assessed by flow cytometry using BCMA-FITC recombinant protein (ACROBiosystems), and performed at days −7, −2 and 0; ii) in-vitro efficacy of CART cells, assessed by the release of interferon-γ by ELISA (R&D) following stimulation with myeloma cell line, and performed at day −2; iii) determination of the vector copy number (CPN) by real-time (RT)PCR of the transduced cells' genomic DNA at day −2; iv) the absence of replication competent retrovirus (RCR) at day −2 is confirmed by PCR analysis of the transduced cells' genomic DNA using GALV primers set, as detailed in 3; v) the characterization of the different cellular subsets was performed at day 0 by flow cytometry, using antibodies mixture as follows: anti-CD3 (Beckman Coulter), anti-CD4 (Biolegend), anti-CD8 (BD), anti-CD56 (Biolegend), anti-CD19 (Biolegend), and anti-CD14 (Beckman Coulter); vi) pH of the DP was assessed at day 0. All the tested parameters represent release criteria of the final DP, which specifications are further detailed in Table 5.

In addition, and in compliance with ANNEX 1 guidelines for Advanced Therapy Medicinal Products (ATMPs), IP and EOP HBI0101 cells are tested for sterility according to the timeline detailed in FIG. 12B. Sterility testing were performed by an outsourced GMP-accredited institution (HyLabs).

### HBI0101 CART In Vivo Detection

Blood was collected from MM or AL patients, prior to and following HBI0101 cells infusion, at the designated times. Genomic DNA was extracted and purified, using the Qiagen QIAamp DNA Blood Mini Kit. CAR copy number (CPN) was determined by quantitative real-time PCR using the Taqman-based primers as follows: MSGV1 primers (Forward: CGGCAGCCTACCAAGAACA, as denoted by SEQ ID NO: 29; reverse primer: TGTGTCGCCGACTCGGTAA as denoted by SEQ ID NO: 30; probe: CGGTGGTACCTCACC, as denoted by SEQ ID NO: 31), and the TaqMan Fast advance Master Mix (Applied Biosystems). Standard curves of MSGV1 plasmid (ranging from 10⁷-10⁰ copies) was generated by serial dilution of the MSGV1 plasmid. The number of circulating CART in 1 mL of blood was then calculated by extrapolating the number of retroviral copies inserted into each CART cell.

For the determination of vector CPN, DNA was extracted from 20×10⁶ expanded HBI0101-T cells at day 2 to CART-cell infusion (QIAamp DNA Blood Mini Kit). Unknown samples, no template controls and standards were run in triplicate. Average MSGV1-HBI0101 vector CPN per transduced cell (CPN/Td) was calculated by normalizing to the endogenous number of diploid albumin copies, and further adjusted to the percent of transduction.

For the Amyloidosis study, quantification of the MSGV1-based HBI0101 vector CPN was performed using the Applied Biosystems StepOne real-time PCR system. The PCR reaction mix contained 1× Taqman Fast advance Master Mix (Applied Biosystems), Taqman-based primer mix for MSGV1 insert or Albumin amplification, Taqman-labeled probes and sterile nuclease free water. MSGV1 primers/probe as follows: Forward: CGGCAGCCTACCAAGAACA, as denoted by SEQ ID NO: 29; reverse: TGTGTCGCCGACTCGGTAA, as denoted by SEQ ID NO: 30; probe: CGGTGGTACCTCACC, as denoted by SEQ ID NO: 31); Albumin primers/probe as follows: Forward: GAGTCACCAAATGCTGCACAGA, as denoted by SEQ ID NO: 32; reverse: GAACGTATGTTTCATCG, as denoted by SEQ ID NO: 33; probe: ACAGGCGACCATGCT, as denoted by SEQ ID NO: 34. Unknown samples, no template controls and standards were run in triplicate. Average MSGV1-HBI0101 vector CPN per transduced cell (CPN/Td) was calculated by normalizing to the endogenous number of diploid albumin copies, and further adjusted to the percent of transduction.

### Study Design and Patients' Evaluation—for the Amyloidosis Study

At the beginning of 2021, a phase I clinical trial for the treatment of MM was initiated and registered at clinical.gov.il (NCT04720313). This study aimed at evaluating HBI0101 safety and efficacy in MM patients and additional plasma cell dyscrasias, including AL. Patients enrolled had to be refractory to at least three lines of treatment including a proteasome inhibitor, an immuno-modulator (IMiD) and an anti-CD38 antibody, and to have no other available registered therapy. At study entry all patients had a progressive disease. The phase I first part of the trial consisted of the administration of HBI0101-transduced T cells, at escalating cell doses of 150-, 450- and 800×10⁶ CAR+ cells. The patients reported herein, each participated in a different safety cohort. The complete study protocol, outlined in FIG. 10, was authorized by the Hadassah Medical Center institutional review board (IRB) and by the Israeli Ministry of Health central ethics committee. A written informed consent from each of the patients was obtained, and the study was conducted and approved in accordance with the ethical guidelines of the Declaration of Helsinki, under the auspices of the Hadassah IRB. One patient was treated on a compassionate basis after obtaining informed consent and authorization of the IRB due to a concomitant active malignancy (myelodysplastic syndrome). This patient was treated with a dose of 450×10⁶ cells as at the time of treatment the safety of cohort III (800×10⁶ cells) was not yet assessed. Adverse events were graded according to National Cancer Institute Common Terminology Criteria for Adverse Events (NCI CTCAE), version 4.03. Cytokine release syndrome was graded according to the published criteria [Lee D W, et al. Biol Blood Marrow Transplant 2019; 25(4)].

### Bone Marrow Immunophenotyping

Bone marrow (BM) aspirates were collected from patients with multiple myeloma and/or AL before and a month after HBI0101 infusion. BM samples were labeled with α-hCD38 (A07778), α-hCD138 (B37788; Beckman Coulter) and α-hBCMA (19F2; Biolegend), and further analyzed using the 10-colors Navios flow cytometer (Beckman Coulter). Flow cytometry analyses were performed using KALUZA software. For the Amyloidosis study, the samples were lysed with IOTest 3 Lysing Solution ×1 (Beckman Coulter); washed before performing flow cytometry.

Serum dFLC and Serum Soluble BCMA (sBCMA) Quantification

Levels of involved and uninvolved FLCs (Siemens FLC assay) were determined in the serum of patients (in the MM and the AL studies) at the indicated time points.

Serum samples for serum BCMA (sBCMA) were diluted 1:1,000 and were analyzed by sBCMA ELISA kit (R&D Systems). The ELISA plates were analyzed using a plate reader set to 450 nm (Biotek Industries) with Gen5 3.05 software. Values represent the mean of duplicate samples of each specimen. Assay Range: 15.6-2000 μg/mL; Assay sensitivity validated by the user: 8.28 pg/mL.

### Multiplex Analysis

Sera from MM patients at baseline (day −10) and at Tₘₐₓ (day of CART peak concentration (Cₘₐₓ)) were processed with the Luminex Performance Human XL Cytokine Magnetic Panel (25-Plex) FCSTM18-26 (R&D), and further assessed by Luminex MAGPIX Instrument with xPONENT 4.2. The multiplex panel included the following cytokines/chemokines: CCL2, CCL3, CCL4, CXCL10, IL-8, G-CSF, GM-CSF, IFN-α, IFN-γ, IL-10, IL-12, IL-13, IL-15, IL-17, IL-1b, IL-1ra, IL-2, IL-4, IL-5, IL-6, IL-7, CCL5, TNF-α, VEGF and FGF. Precision and sensitivity data for these parameters is reported in the manufacturer's manual. Data was analyzed using MILLIPLEX® Analyst 5.1 Software. The concentration of IFN-γ, TNF-α and IL-2, was determined by ELISA (R&D).

### T-Cell Exhaustion/Activation Following Antigen Stimulation and at Rest

To evaluate T-cell exhaustion/activation following antigen stimulation, anti-BCMA chimeras were cultivated for 24 hours either in the presence of the H929-MM cell line, or BCMA-overexpressing K562 (K562-BCMA), or K562 cell line in T-cell medium without IL-2. Co-cultures were performed in a 96-round bottom wells plate. Co-culture of 0.05×10⁶ of CAR+ effector T cells were mixed with 0.05×10\hat{ }6 Target cells (E:T ratio of 10:1) in T-cell medium without IL-2 and incubated for 24 hours at 37° C. Following incubation, cells were washed, stained at 4° C. for 20 min with a mixture of the following fusion protein/antibodies: BCMA-FITC (Acro), anti-CD3 (Beckman Coulter), anti-PD-1 (Beckman Coulter), anti-LAG-3 (eBioscience), anti-TIM-3 (eBioscience), anti-TIGIT (eBioscience) for the assessment of T-cell exhaustion, and with a mixture of the following fusion protein/antibodies: BCMA-FITC (Acro), anti-CD137 (4-1BB) (Beckman Coulter), anti-CD69 (BD Pharmingen), anti-CD25 (Beckman Coulter) for the assessment of T-cell activation. Cells were washed twice with FACS buffer. Samples were acquired on a Navios cytometer. Data were analyzed using Kaluza software. Cells were gated on BCMA CAR+.

To assess T-cell exhaustion/activation at rest, anti-BCMA chimeras were cultured for 12 days post transduction in T-cell media supplemented with 3001 U/mL IL-2. Cells, without prior antigen stimulation, were stained every two-four days with T-cell exhaustion and activation antibodies mixture described above.

### Established Tumor Assay

About 6-12 weeks year-old NOD/SCID/Gamma mice were subcutaneous injected with 4×10⁶ NCI H929 cells resuspended in 100p HBSS medium (Biological Industries, Beth Haemek, Israel) and 100 μl Cultrex matrix (Trevigen, MD). Injections of transduced lymphocytes resuspended in 200 μl HBSS medium were performed after tumor inoculation. Tumor size was measured every 2-3 days using a caliper in a blinded fashion or assessed by injecting luciferin solution (5 mg/ml, 200 μl/mouse, Promega) and bioluminescence imaging (BLI) evaluation. Animals were humanely euthanized if the tumor exceeded 16 mm in diameter. All the procedures were performed according to the guidelines of the university committee for animal welfare.

### T-Cell Persistence in the Blood of NSG Mice

Twenty-five microliters of blood were collected from the tail vein of the mice, lysed with IOTEST 3 Lysing Solution ×1 (Beckman Coulter) for 10 mins, and stained with a mixture of recombinant human BCMA protein (Active) (FITC) (Abcam, ab246085), anti-CD3 (Beckman Coulter), anti-CD8 (BioLegend), and anti-CD4 (BioLegend), CD3+BCMA.CAR+ cells were referred to as “CAR T cells.”

### Immunohistochemistry

Four micron-thick Formalin-Fixed Paraffin-Embedded (FFPE) tumor sections were stained with the anti-human CD3 antibody (103A-76, 1507011D, CellMarque, diluted 1:1000), on a BenchMark ULTRA autostainer (Ventana Medical Systems) with standard antigen retrieval (CC1 buffer, 40 min). Detection was performed using the ultraView Universal DAB Detection Kit (Ventana Medical Systems).

Co-Culture of BM-MNCs with BCMA.CART Cells

Bone marrow aspirates from patients with different types and stages of plasma cell dyscrasias were obtained in accordance with Helsinki approval of the Ethical Committee of Hadassah Ein Kerem Medical Center. Bone marrow-derived mononuclear cells (BM-MNCs) were isolated by centrifugation over a density gradient medium (Lymphocyte Separation Medium, Lonza) immediately or following overnight incubation at room temperature after BM aspiration. BM-MNCs were co-cultured with H8BB CART effector cells or control non-transduced (NT) T cells, at 37° C. overnight and then analyzed by flow cytometer.

### In-Vitro PCs Elimination

BM mononuclear cells (BM-MNCs) from primary BM samples, isolated by ficoll density gradient centrifugation, were co-cultured 1:1 CART:BM-MNCs cells (10⁵ cells each) for overnight at 37° C. Cells were labeled as above and assessed by flow cytometry.

### In Vitro CD138 PCs Cytotoxicity

BM-MNCs were enriched in CD138 PCs using the EasySep human CD138 Positive Selection Kit II (STEMCELL Technologies). BM-MNCs, CD138, or CD138− fraction of BM-MNCs were cocultured either with HBI0101 or NT effector cells in TCM at a 1:1 E:T ratio for 2 hours (for caspase-3 killing assay) or overnight (for cytokines release assay and 4-1BB activation assay). Cellular fraction was labeled extracellularly with hBCMA recombinant protein, a-CD3, and a-CD137 for T-cell activation assay, or with a-BCMA, a-CD3, and a-CD138, fixed, permeabilized and intracellularly labeled with cleaved caspase-3 for cytotoxicity assay. Stained cells were analyzed by flow cytometer. Cell supernatants were collected and the secretion of IFNγ and TNFα was quantified by ELISA (R&D) according to the manufacturer's instructions.

### CD138 PCs Apoptosis

Cellular fraction of the in vitro PC culture cell suspension was labeled extracellularly with anti-BCMA (Bio-Legend), anti-CD3 (Beckman Coulter), and anti-CD138 (Beckman Coulter) at 4° C. for 20 minutes. Cells were then washed twice in PBS 2% FBS, fixed and permeabilized with Fix/Perm solution (BD), and then intracellularly stained with anti-active caspase-3 antibody (BD Pharmingen). The percent of cleaved caspase-3 in CD3-BCMA gated cells was determined using flow cytometry.

### Non-PCs Viability by 7AAD Staining

BM-MNCs were cocultured either with HBI0101 or NT cells at a E:T ratio of 1:1 for 1 hour at 37° C. in AIM-V (Gibco) supplemented with 5% human AB serum (Access Biological LLC) and 1% Glutamax (Gibco). Following incubation, cells were washed with PBS and stained with anti-CD38 and anti-CD138 (Beckman Coulter) for 20 minutes. 7-AAD (Tonbo Bioscience) was added 5 minutes prior to samples acquisition (10-color Navios flow cytometer). Samples were gated on CD38−CD138− “AND NOT” CART. Samples were analyzed using Kaluza 2.1 software.

4-1BB Activation Assay HBI0101-transduced or NT T cells were cocultured either with BMMNCs, or with the CD138-positive or CD138-negative fractions of BM-MNCs (1:1 E:T ratio), in a U-bottom 96-well plate. Following an overnight incubation at 37_C, cells were labeled with human BCMA recombinant protein (ACRO), anti-CD3 (Beckman Coulter), and anti-CD137 (4-1BB; BioLegend), and then analyzed by flow cytometry. To assess the percentage of 4-1BB CAR cells, samples were gated on live CD3CAR cells, while NT cells samples were gated on live CD3 cells.

### Clinical Study, Inclusion and Exclusion Criteria

### Inclusion Criteria:

≥18 years of age; Voluntarily signed informed consent form (ICF); Eastern Cooperative Oncology Group (ECOG) performance status 0-2; Diagnosis of MM with relapsed or refractory disease and have had at least 3 different prior lines of therapy; including proteasome inhibitor, immunomodulatory therapy and at least one antibody therapy.

Subjects must have measurable disease, including at least one of the criteria below:
Serum M-protein greater or equal to 0.5 g/dL;
Urine M-protein greater or equal to 200 mg/24 h;
Serum free light chain (FLC) assay: involved FLC level greater or equal to 5 mg/dL (50 mg/L) provided serum FLC ratio is abnormal;
A biopsy-proven evaluable plasmacytoma;
Bone marrow plasma cells >20% of total bone marrow cells;
Non secretory patient will be allowed provided they have measurable disease by PET-CT or bone marrow aspiration, as designated.
Women of child-bearing potential (WCBP), must have a negative serum pregnancy test prior to treatment. All sexually active WCBP and all sexually active male subjects must agree to use effective methods of birth control throughout the study;
Recovery to ≤Grade 2 or baseline of any non-hematologic toxicities due to prior treatments, excluding alopecia and Grade 3 neuropathy;
Ability and willingness to adhere to the study visit schedule and all protocol requirements.

### Exclusion Criteria:

Any prior systemic therapy for MM within 14 days prior to leukapheresis.
Long-acting growth factors or drugs used for cell mobilization within 14 days prior to leukapheresis.
Short-acting growth factors or drugs used for cell mobilization within 5 days prior to leukapheresis.
Therapeutic doses of steroids within 3 days prior to leukapheresis (Physiological replacement doses of steroids are allowed up to 12 mg/m2/d hydrocortisone or equivalent).
Any prior systemic therapy for MM within 14 days prior to the start of lymphodepletion.
CNS directed radiation within 8 weeks prior to lymphodepletion.
Long-acting growth factors or drugs used for cell mobilization within 14 days prior to lymphodepletion.
Short-acting growth factors or drugs used for cell mobilization within 3 days prior to lymphodepletion.
Investigational cellular therapies within 8 weeks prior to lymphodepletion.
Subjects with known bulky central nervous system disease.
Inadequate hepatic function ALT>3 normal value.
Inadequate renal function CRCL<20.
International ratio (INR) or partial thromboplastin time (PTT)>1.5×ULN, unless on a stable dose of anticoagulant for a thromboembolic event consistent with exclusion.

Inadequate bone marrow function defined by absolute neutrophil count (ANC)<1000 cells/mm3, platelet count <30,000 mm3, or
hemoglobin <8 g/dL (Blood transfusions are allowed), absolute lymphocyte count <500 cells/mm3.
Left ventricular ejection fraction <40%.
Ongoing treatment with chronic immunosuppressants.
Presence of active infection within 72 hours prior to lymphodepletion.
Previous history of an allogeneic bone marrow transplantation or treatment with any gene therapy-based therapeutic for cancer.
Significant co-morbid condition or disease which in the judgment of the Inventors would place the subject at undue risk or interfere with the study.
Known human immunodeficiency virus (HIV) positive status.
Active Hepatitis B or Hepatitis C infection (see acceptance criteria for HBV, HCV and HIV).
Subjects with a history of stroke, unstable angina, myocardial infarction, or ventricular arrhythmia requiring medication or mechanical control within 3 months.
Subjects with second malignancies in addition to myeloma, if the second malignancy has required therapy in the last 2 years or is not in complete remission; exceptions to this criterion include successfully treated non-metastatic basal cell or squamous cell skin carcinoma, or prostate cancer that does not require therapy.
Subjects who have had a venous thromboembolic event requiring anticoagulation and who meet any of the following criteria:
Have been on a stable dose of anticoagulation for <1 month (except for acute line insertion induced thrombosis);
Have had a Grade 2, 3, or 4 hemorrhage in the last 30 days
Are experiencing continued symptoms from their venous thromboembolic event (e.g. continued dyspnea or oxygen requirement);
Pregnant or lactating women.

TABLE 1
Patients' previous treatments: exposures and refractoriness

| | | | |
|---|---|---|---|
| Total | Cohort 1 | Cohort 2 | Cohort 3 |
| N = 20 | n = 6 | n = 7 | n = 7 |

PROTEASOME INHIBITOR
BORTEZOMIB

| | | | | |
|---|---|---|---|---|
| EXPOSED | 19(95%) | 6(30%) | 6(30%) | 7(35%) |
| REFRACTORY | 17(85%) | 6(30%) | 4(20%) | 7(35%) |

CARFILZOMIB

| | | | | |
|---|---|---|---|---|
| EXPOSED | 11(55%) | 4(20%) | 5(25%) | 2(10%) |
| REFRACTORY | 10(50%) | 4(20%) | 5(25%) | 1(5%) |

IXAZOMIB

| | | | | |
|---|---|---|---|---|
| EXPOSED | 2(10%) | 0(0%) | 2(10%) | 0(0%) |
| REFRACTORY | 2(10%) | 0(0%) | 2(10%) | 0(0%) |

IMIDS

| | | | | |
|---|---|---|---|---|
| LENALIDOMIDE | | | | |
| EXPOSED | 18(80%) | 6(30%) | 5(25%) | 7(35%) |
| REFRACTORY | 18(80%) | 6(30%) | 5(25%) | 7(35%) |

THALIDOMIDE

| | | | | |
|---|---|---|---|---|
| EXPOSED | 1(5%) | 0(0%) | 19(5%) | 0(0%) |
| REFRACTORY | 1(5%) | 0(0%) | 1(5%) | 0(0%) |

POMALIDOMIDE

| | | | | |
|---|---|---|---|---|
| EXPOSED | 17(85%) | 5(25%) | 5(25%) | 7(35%) |
| REFRACTORY | 17(85%) | 5(25%) | 5(25%) | 7(35%) |

ANTI-CD38
DARATUMUMAB

| | | | | |
|---|---|---|---|---|
| EXPOSED | 19(95%) | 6(30%) | 6(30%) | 7(35%) |
| REFRACTORY | 19(95%) | 6(30%) | 6(30%) | 7(35%) |

ISATUXIMAB

| | | | | |
|---|---|---|---|---|
| EXPOSED | 1(5%) | 0(0%) | 1(5%) | 0(0%) |
| REFRACTORY | 1(5%) | 0(0%) | 1(5%) | 0(0%) |

ANTI-BCMA (BELANTAMAB
MAFODOTIN)

| | | | | |
|---|---|---|---|---|
| EXPOSED | 9(45%) | 2(10%) | 5(25%) | 2(10%) |
| REFRACTORY | 9(45%) | 2(10%) | 5(25%) | 2(10%) |

All data are presented as number of patients, percent of patients (no., %)

### Statistical Analysis

Statistical analyses were performed using GraphPad Prism (V9.1.0). Unless otherwise stated, paired Student's t-test or two-way ANOVA tests were used for normal data at equal variance. P<0.05 was considered significant and is marked as * in the figures. T cells from at least three different healthy donors were used for all in vitro and in vivo experiments. Kruskal-Wall is analysis with Dunn's post hoc analysis was performed for mouse tumor volumes. Analysis of variance or log-rank (Mantel-Cox) test for survival data were performed.

For the purpose of comparison between the different HBI0101 doses for both safety and short-term efficacy (clinical trial NCT04720313), a sample size of 20 patients was planned. Descriptive statistics was performed using median with range for continuous variables and counts and percentages for categorical variables. OS was defined as the time from randomization until death from any cause and is measured in the intent-to-treat. PFS was defined as the time from CART infusion until disease progression or death from any cause. OS and PFS were estimated using the Kaplan-Meier method. Overall response rate (ORR) was defined as the proportion of patients achieving either a stringent complete response (sCR), a complete response (CR), a very good partial response (VGPR) or a partial response (PR). Descriptive statistics were performed using percentages. For the purpose of this phase I data analysis, data was collected until February 3rd 2022. Data were compiled using Excel software and all statistical analyses were performed using GraphPad Prism (v9.2.0) software. Appropriate statistical methods were used to calculate significance, as described in the brief description of the figures (two-way ANOVA and paired or unpaired t-test).

### Example 1

### Generation of Different Anti-BCMA CARs and Preliminary Evaluation

Based on an identical scFv derived from the C11D5.3 antibody [2], three second-generation anti-BCMA CARs were constructed. As co-stimulatory molecules, these CARs incorporate either an intra-cellular domain derived from 4-1BB (for H8BB and ICBB) or CD28 (for IC28) (FIG. 1A). More specifically, H8BB includes hinge and transmembrane (TM) domains derived from CD8α, while these were replaced by an IgG4 hinge and a CD28 TM in the ICBB. IC28 shares the same hinge and CD28 TM domain as ICBB CAR, but its co-stimulatory domain was switched from 4-1BB to CD28 (FIG. 1A). As a reference, the inventors used the previously described [2] and in clinically tested [3]H828 CAR, which incorporates a CD8α-derived hinge-TM and CD28 as costimulatory domain.

The function of the BCMA-specific CARs was next evaluated. To that end, anti-CD3-stimulated human PBMCs were transduced with these different CARs. As shown in FIG. 1B, BCMA CAR molecules were expressed at high levels, ranging from 66% for ICBB CAR to 81% for H8BB CAR. Then, to evaluate BCMA CARs basic function, an overnight co-culture of CAR-transduced T-cells with target cells (K562 vs. K562-BCMA) were performed and the IFNγ secretion was measured by ELISA. FIG. 1C shows that all four anti-BCMA CARs were able to mediate IFNγ release. These results confirm that the different anti-BCMA CARs can be expressed at the T-cell surface (FIG. 1B) and mediate antitumoral function in those cells (FIG. 1C). Yet, although IC28 was able to induce cytokine release upon specific stimuli, this molecule was less efficient than ICBB-, H8BB- and H828 CARs in mediating cytokine release (FIG. 1C, IC28 vs. CD34t, ns), in killing assays and at upregulating activation markers upon antigen stimulation (data not shown). In addition, FIG. 1C shows that H8BB CAR T cells secrete less IFNγ than corresponding ICBB CAR T cells (p<0.05), when cultured in the presence of the BCMA-negative control cell line K562. Since both chimeras share identical scFv and co-stimulatory domains, this observation may indicate that the hinge and TM moieties incorporated into these CARs may affect their “off-target” specificity. Given these preliminary data and based on emerging studies demonstrating 4-1BB co-stimulation benefits [11, 12], the inventors therefore focused on 4-1BB-based CARs, namely ICBB and H8BB.

### Example 2

Growth and Viability of T Cells Transduced with the Different Anti-BCMA Chimeras

The ability of the different anti-BCMA CARs to promote T-cell proliferation over the weeks in culture was next assessed. To this end, the growth of the different BCMA CAR-transduced T cells was monitored over 18 days of culture. FIG. 1D shows that ICBB CAR T cells display a slower expansion profile in comparison with H828-, H8BB- and CD34t-CAR T cells (ICBB vs. H8BB/H828/CD34t, p<0.05). Additionally, BCMA CAR T cells viability was also assessed by determining the proportion of dead cells by staining transduced lymphocytes with propidium iodide (PI). FIG. 1E indicates that a large proportion of ICBB-transduced cells were positive for PI (61.4%±2.0) compared to the significantly reduced number of PI-positive cells detected in all the other groups (from 5.3-9.1%; p<0.0001). This data indicates that H8BB-transduced cells have a similar ability to expand in vitro over time as H828-transduced cells, while ICBB-transduced cells exhibit an increased cell death (FIG. 1E) and a subsequent reduced ability to expand (FIG. 1D).

### Example 3

### Cytokine Secretion by BCMA-Specific CARs

Following the initial evaluation of CAR functionality, wider cytokine secretion assays were conducted by co-culturing BCMA-CAR transduced T-cells with different plasma and MM cell lines. In general, BCMA-CAR T cells could specifically secrete high levels of cytokines (IFNγ, TNFα and IL-2) important for anti-tumor immunity [Wilde S, et al. J Immunol. 189(2):598-605. (2012)], when compared to CD34t negative control CAR T cells (FIG. 2; p<0.05). More precisely, when performing a co-culture with target RPMI8226, an average secretion of 6721 μg/ml of IFNγ mediated by the H8BB CAR, 4520 μg/ml by ICBB CAR and 5055 μg/ml by H828 CAR, was observed. Similar results were observed when measuring TNFα and IL-2 secretion (for example, an average of 970 μg/ml of IL-2 for ICBB CAR and 1450 μg/ml for H8BB CAR and 260 pg/ml for H828 CAR using RPMI-8226 target). The difference between H8BB and H828 was found statistically significant (for IFNγ, p=0.00098, for IL-2 p=0.01 and for TNFα-p=3.7×10⁻⁵; calculated using a paired Student's t-test). As previously described herein (FIG. 1B), it is noteworthy that H8BB- and H828-CAR T cells displayed the lowest non-specific cytokine secretion in control co-cultures with an antigen-negative target (K562) or without any target (FIG. 2A-2C). This observation reinforces the idea that “off-target” (i.e., unspecific target) and tonic (i.e., without antigen stimulation) signaling may be influenced by the CAR configuration.

### Example 4

### Phenotypic Analysis of BCMA-Engineered CAR T-Cells

The inventors then sought to characterize the phenotype of BCMA-CAR T-cells. The inventors focused on the CD4/CD8 ratio and on the memory differentiation pattern. To this end, BCMA-CAR transduced T-cells were sampled three weeks following PBMCs' activation and analyzed for marker expression by flow cytometry. As seen in FIG. 3A, no substantial differences were noticed between the different CAR-expressing populations, as to the CD4/CD8 ratio, which was approximately 1:2. Moreover, when analyzing the expression of the memory markers CD45RO and CCR7, a proportion of 44/40% of central memory (CM—CD45RO⁺/CCR7⁺) and 33/39% of effector memory (EM—CD45RO⁺/CCR7⁻) T-cells was observed in the ICBB- and H8BB-CAR T-cells, respectively (FIG. 3B). However, H828-transduced T cells displayed a higher proportion of effector memory T-cells compared to 4-1BB-based CARs (57% vs 36%; p=0.037) (FIG. 3B). It has been reported that CAR T cells displaying a “less differentiated profile” (namely, central memory cells), persist and, thus, perform better in vivo [Ren H, et al. Front Immunol. 12:745109. (2021)]. In addition, it has been also reported that exhausted T cells may become progressively dysfunctional, and that loss of function is mediated by the upregulation of inhibitory receptors such as, programmed death receptor-1 (PD-1), lymphocyte activation gene-3 (LAG-3), T cell immunoglobulin-3 (TIM-3), and T cell immunoreceptor with Ig and ITIM domains (TIGIT) [Anderson A C, et al. Immunity. 44(5):989-1004. (2016)]. Therefore, the expression of these exhaustion markers was monitored in the different BCMA-CAR T cells over time in culture. FIG. 3C(i)-(iv) shows that there is a marked increase in the expression level of PD-1 (FIG. 3C(i)), LAG-3 (FIG. 3C(ii)), TIM-3 (FIG. 3C(iii)) and TIGIT (FIG. 3C(iv)) on the surface of H828-transduced cell when compared to 4-1BB-based CARs over time in culture (p<0.001). It is noteworthy that H828-transduced T cells exhibit significantly high levels of exhaustion markers at very early stages of the culture following transduction. This elevation in the basal expression of exhaustion markers may be mediated by tonic signaling augmented by the CD28 co-stimulatory portion of the CAR, and, importantly, may impede with CAR T-cell function in vivo.

### Example 5

### Upregulation of Activation Markers and Exhaustion Profile of Anti-BCMA Chimeras Following Antigen Stimulation

To further investigate whether the observation that high basal expression of exhaustion markers on the surface of H828-based CAR T cells may affect their functionality, while a reduced expression level of these markers could ameliorate T-cell function of the 4-1BB-based CAR T cells, the expression of PD-1, LAG-3, TIM-3 and TIGIT on the surface of the different BCMA CAR T cells was analyze following overnight co-culture with BCMA-positive targets. FIG. 4A(i)-(iv) indicates that all the four exhaustion markers are highly expressed on the surface of H828 CAR-T cells, even in the absence of target, as previously shown in FIG. 3C. On the contrary, 4-1BB-based CAR T cells responded specifically to the different stimulations, by upregulating PD-1 (FIG. 4A(i)), LAG-3 (FIG. 4A(ii)) and TIM-3 (FIG. 4A(iii)) upon BCMA-mediated specific stimulation. The only exception is for the inhibitory receptor TIGIT (FIG. 4A(iv)) which was found basically lower in 4-1BB-based CAR T cells in comparison with H828-CAR T cells but did not respond to antigen stimulation. It is possible that this marker requires a longer stimulation. Interestingly, the basal expression of TIM-3 in ICBB-CAR T cells was significantly higher than in H8BB CAR T cells (p<0.05). Given the fact that TIM-3 expression can also be induced upon activation [Han G, Front Immunol. 4:449 (2013)], this observation may account for the non-specific activity observed in cytokine release (FIGS. 1C and 2A-2B).

Next, to test the hypothesis that high basal expression of exhaustion markers may impede on CAR T cells function (FIG. 3C), the inventors examine whether these CARs could mediate the upregulation of T-cell activation markers upon antigen stimulation. Following an overnight or a 4-hour co-culture with BCMA-positive target cells, BCMA-CAR T-cells were analyzed for surface expression of activation markers including 4-1BB (i.e., CD137), CD25 and CD69. As anticipated, a correlation between the activation status and the exhaustion profile of CAR T cells was found. Indeed, FIG. 4B ((iii)), indicates that H828-CAR T cells upregulate their 4-1BB molecules following stimulation with BCMA-expressing targets but to a lesser extent than 4-1BB-based CAR T cells (H828 vs. ICBB and H8BB, p<0.05). Similar results were achieved when measuring CD69 expression following short co-cultures (4 hours) of myeloma target cells (FIG. 4B (ii)). CD25 expression (FIG. 4B (i)) shows, that H828 CAR T cells are highly activated in comparison with H8BB CAR T cells (H828 vs. H8BB, p<0.001), even when they are unstimulated (“No target”) or stimulated with BCMA-negative cells (K562). In addition, following stimulation with specific target, H8BB CAR T cells significantly upregulate CD25 (p<0.01), while ICBB- and H828 CAR T cells did not respond significantly to antigen stimulation (FIG. 4B (i)). For example, whereas CD25 ex-pression levels were generally higher following co-culture with BCMA-positive targets in H828-CAR T cells (between 62-65%) in comparison with H8BB-CAR T cells (52.9-58.6%—H828 vs. H8BB, P<0.001), they did not increase significantly from those observed for unstimulated cells or in co-culture with BCMA-negative cells (59.3-60.8%). Furthermore, and in line with TIM-3 elevation detected in ICBB CAR T cells (FIGS. 3C and 4A), FIG. 4B shows that those cells express high levels of 4-1BB (p<0.001) when stimulated with the non-specific cell line K562. These results indicate that H8BB exhibited the lowest levels of exhaustion receptors and increased upregulation of activation markers which may facilitate a better specific and long-term in vivo activity. H8BB CAR therefore appears to be the safer and more effective configuration in the context of MM-targeting.

### Example 6

### H8BB-CAR Mediates Improved In Vitro Cytotoxicity and In Vivo Biological Activity

To assess the assumption that H8BB CAR is safe and efficient at mediating tumor eradication, the cytotoxic activity of T-cells transduced with the different BCMA-CARs was next examined. To that end, tumor target cells were labeled with Carboxyfluorescein succinimidyl ester (CFSE) and co-cultured with BCMA CAR- or CD34t control T cells for 4 hours. Then, CFSE-positive cells were analyzed for PI staining, as a surrogate for cytotoxic activity. As observed in FIG. 5A, all CAR constructs mediated significant cytotoxicity against the H929 and RPMI-8226 myeloma cell lines, in comparison with CD34t control T-cells (p<0.0001). Moreover, 4-1BB-based CAR T cells demonstrated significantly higher cytotoxicity against RPMI-8226 myeloma cells even at the lowest ratio 2.5:1 (ICBB or H8BB vs. CD34t, p<0.001). Similar results were obtained when H8BB CAR T cells were co-cultured with H929 (FIG. 5A(iii); H8BB vs. CD34t, p<0.05). Additionally, H8BB CAR, but not ICBB, was more efficient than H828 CAR at mediating cytotoxicity against RPMI-8226 at escalating E:T ratios (FIG. 5A(ii); H8BB vs. H828 both at 5:1 and 10:1 ratios, p<0.05). As expected, CAR T-cells did not show any significant cytotoxicity against the BCMA-negative cell line K562 (FIG. 5A(i)) when compared to corresponding control T-cells. These results suggest that H8BB CAR displays an advantage over H828 and ICBB CARs at mediating myeloma cells elimination in vitro, even at low E:T ratio. Since cytotoxicity is mediated via the spatial recognition between the CAR molecule on the effector cells and cognate antigen at the surface of the target cells, it can be speculated that the configuration of H8BB CAR at the T-cell surface is optimal to allow BCMA recognition and induce myeloma killing.

Finally, the BCMA-CARs' function in vivo, was analyzed using a xenograft model of human tumors. Immunodeficient NSG mice were inoculated with 4×10⁶ NCI-H929 cells. Five days later, these mice were adoptively transferred intravenously either with BCMA-CAR or control T-cells (CD34t-transduced T cells) or were left untreated (“No treatment”). Tumor volume was blindly evaluated every 2-3 days. FIG. 5B indicates that the mice infused with H8BB- or H828-CAR T cells (FIG. 5B(v), 5B(iv), respectively) display a significant delay and/or a marked regression in the tumor volume (H8BB vs. control groups, p<0.0001 and H828 vs. control groups, p<0.001), when compared to the control groups (“No treatment” and or CD34t groups (FIG. 5B(i), 5B(ii), respectively)) or ICBB group (FIG. 5B(iii)). This observation is also reflected by the improved overall survival (FIG. 5C; H8BB and H828 groups vs. control groups, p<0.0001; by LogRank analysis). Furthermore, a significant reduction was found in the tumor volume of H8BB CAR-treated mice in comparison with the tumor volume of H828 CAR-treated cohort (H8BB vs. H828, p<0.05; by Kruskal-Wallis test, with Dunn's post hock analysis). Puzzlingly, ICBB CAR T cells showed no significant anti-tumor activity in comparison with the control groups. Thus, the inventors conclude that, while ICBB CAR could not mediate myeloma eradication in vivo, H8BB CAR was proven statistically more efficient than H828 CAR in mediating significant anti-tumor cytotoxicity both in vitro and in vivo. It is noteworthy that out of the 9 mice that were administered either with H8BB- or H828-CAR T cells, 4 mice in the H828 CAR group relapsed, and two additional mice showed fluctuations in their tumor volumes, while only 2 mice in the H8BB group experienced relapses (FIG. 5B). Although this data is not directly reflected in the median survival between the H8BB and H828 groups (FIG. 5C), it implies that at long term, H8BB CAR T cells may persist longer than H828 counterparts (i.e., less exhausted, less non-specific activation, less tonic signaling), and thereby prevent relapses.

### Example 7

### Further Evaluation of the Antitumor/Off-Target Activity of H8BB CAR T Cells In Vivo

Given the several advantages of H8BB CAR on the other BCMA CARs presented here ((i.e., appropriate optimal cell growth (FIG. 1C), antitumor cytokines production with a minimal non-specific secretion (FIG. 2A-2C), reduced exhaustion profile (FIG. 4A), reduced tonic signaling and non-specific activation (FIG. 4B), high antitumoral cytotoxic activity both in vitro and in vivo (FIG. 5A-5C), all efforts were therefore focused at evaluating H8BB CAR potential therapeutic value to target BCMA in the treatment of plasma cells malignancies. In this attempt, the inventors opted to study the anti-tumor function mediated by H8BB CAR T cells and aimed at determining the optimal effective dose of H8BB CAR T cells for infusion to achieve a desired therapeutic effect. To that end, an additional model was established in which NSG mice were injected with 4×10⁶ luciferase-expressing NCI-H929 myeloma cells. In the experiment described in FIG. 6, it was decided to challenge H8BB CAR T cells by subjecting them to a higher tumor load than detailed above in FIG. 5B-5C. Only once the tumors were well-established (164±30 mm³, about two weeks following myeloma inoculation), the mice were infused with escalating doses of H8BB CAR T cells (5, 10 or 15×10⁶ cells) or non-transduced (NT) T cells as control. As shown in FIG. 6, H8BB CAR T cells exhibit a strong anti-myeloma effect. Specifically, the mice groups that received a single injection of 10 or 15×10⁶ H8BB CAR T cells, showed an initial increase in tumor volume followed by a rapid and complete tumor regression by 2 to 3 weeks post infusion (FIGS. 6A(i)-6A(iv), 6C). In the mice group that received 5×10⁶ CAR T-cells, 4/5 mice showed complete tumor regression except for one that initially displayed higher tumor load at the day of CAR T injection (FIGS. 6A, 6C). Survival curve is shown in FIG. 6D. These results indicate that the minimal effective dose for therapeutic effect against NCI-H929 ranges between 5- to 10×10⁶ cells. As for the control group (treated with NT T cells), the mice were all sacrificed within a month post tumor inoculation for ethical reasons. In parallel, the concentration of soluble BCMA was evaluated in the serum from mice treated in this experiment as a biomarker for MM [Ghermezi M. Haematologica (2017); 102:785-795] and as evidence for tumor eradication. As seen in FIG. 6B(I)-6B(iv), sBCMA levels rapidly declined in concomitance with tumor regression following H8BB CAR T-cell infusion. Also, this data confirms that high concentrations of sBCMA in the blood of NSG xenografts did not seem to block the anti-myeloma activity of H8BB CAR T cells in vivo.

To investigate the persistence of the H8BB CAR T cell treatment in NSG myeloma xenografts, the presence of CAR T cells both intratumorally and in the blood was evaluated. FIG. 6E shows that H8BB CAR+ T cells were detected in the peripheral blood of myeloma NSG xenografts three days after T-cell injection, significantly increased 13-22 days after adoptive transfer and then declined over the next three weeks. In a parallel cohort of NSG MM xenografts, mice were treated with 7.5×10⁶ H8BB-CAR T-cell or control (NT) T-cell and sacrificed at day 9 following T-cell administration (i.e., day 20 following tumor inoculation). Tumors were excised and weighed. FIG. 6F indicates that the average weight of the tumors from H8BB CAR T-treated mice was significantly lower than in the NT control group (p=0.03). Immunohistochemistry analysis performed on tumor sections for the human T cell marker CD3, shows that human T cells were abundantly present in the H8BB sections (FIG. 6G, bottom panel). In contrast, staining of the tumor sections of the control NT mice group revealed only sparse stained areas (FIG. 6G, upper panel). Similar results are also shown in FIG. 8A-8C. Altogether, these data demonstrate that, following their infusion into the NSG mice tail vein, H8BB CAR T actively proliferate and traffic to the tumor site where they exert a strong antitumor effect leading to tumor eradication.

### Example 8

Anti-Myeloma Efficacy of H8BB CAR T Cell Against Primary Cells from Multiple Myeloma Patients

As a preclinical evaluation of H8BB CAR T cells in myeloma patients, BCMA expression was assessed by flow cytometry on plasma cells from the bone marrow of patients suffering from different plasma cells pathologies (e.g., multiple myeloma—MM, amyloidosis—AL, Monoclonal gammopathy of undetermined significance—MGUS, plasmacytoma—PC and Waldenstrom's macroglobulinemia—WDS). In line with the data reported previously [Bal S, Sigler A, Blood. 134(Suppl 1):S5452 (2019)], a differential median expression of BCMA by the plasma cells (gated on CD38⁺⁺CD138⁺⁺) derived from the bone marrow of MM, AL, MGUS, PC and WDS patients, with an MFI of 3.8±3.8, 1.2±0.9, 1.3±0.4, 1.9±1.1 and 1.5±0.4 respectively (FIG. 7A), was observed. Although the average of BCMA mean fluorescence intensity (MFI) in MM patients is markedly higher in comparison with other plasma cell disorders, p values do not meet significance. Given the efficacy of the BCMA CAR T therapy described herein in a xenograft model of multiple myeloma cell line, the inventors sought to investigate whether H8BB-CAR T-cells will be efficient in targeting primary myeloma cell co-cultures. To this end, bone marrow-derived mononuclear cells (BM-MNCs) from MM patients were co-cultured with either H8BB CAR T cells or NT (control) in an allogeneic system (FIG. 7B-7D). FIG. 7B indicates that H8BB T cells significantly upregulate their 4-1BB expression compared to NT cells, when co-cultured with BM-MCs of MM patients (p=0.027). FIG. 1C shows the level of BCMA expression on patients' BM-MNCs (gated on CD38⁺CD138⁺). Overnight co-incubation of BM-MNCs isolated from MM patients with H8BB CAR T cells resulted in the almost complete elimination of the plasma cells (gated on CD38++CD138++; FIG. 7D, lower panel). In contrast, plasma cells were not affected by the presence of control NT cells (FIG. 7D, upper panel), demonstrating the specificity of BCMA-CAR targeting. In addition, the expression of CD137 (4-1BB), as a marker of T cell activation was examined. Altogether, these data confirm the potential efficacy of H8BB CAR T-based therapy for the treatment of multiple myeloma.

### Example 9

### Clinical Study in MM Patients—Evaluation of the Clinical Applicability of the CAR T of the Invention

A clinical study has been initiated in February 2021, enrolling R/R malignant plasma cell patients to be treated with autologous H8BB-CAR T-cells (NCT04720313) in a dose-escalation study evaluating first the safety and then the efficacy of this approach.

A non-randomized, open label, single-site Phase 1 clinical study is therefore conducted at the Department of Bone Marrow Transplantation and Cancer Immunotherapy, Hadassah Medical Center (HMO), to evaluate the therapeutic applicability of the CAR T (H8BB, the CAR molecule of the present disclosure is also indicated herein in all clinical trials as HBI0101) molecule of the present disclosure. The structure of the HBI0101/H8BB CAR molecule is disclosed in FIG. 9. The complete study protocol and design are detailed in FIGS. 10 and 11.

Subjects elected in accordance with the exclusion and inclusion criteria specified in the experimental procedures undergo lymphopheresis at day −10 prior to infusion, to provide starting material for the preparation of the CAR T cells. Collected cells are delivered to the GMP facility for further stimulation, transduction and expansion (FIG. 12A-12B). Patients' lymphodepletion before HBI0101 infusion is achieved by the administration of fludarabine 25 mg/m2 and cyclophosphamide 250 mg/m2 on days −5 to −3 (FIG. 10). Patients with creatinine clearance <30 ml/min receives bendamustine at a dose of 90 mg/m2 on days −4 and −3. Fresh HBI0101 cells are administered at escalating doses of 150—(cohort 1), 450—(cohort 2) and 800×10\hat{ }6 (cohort 3) CAR+ cells. Following the infusion, patients remain hospitalized for at least 10 days as a protocol requirement. Patients are followed for adverse events (AEs) daily during the hospitalization period and for safety and efficacy at a predefined schedule detailed in FIG. 10.

### Multiple Myeloma Clinical Monitoring

Response to HBI0101 is evaluated at a predefined time schedule according to IMWG criteria [Kumar S, et al. Lancet Oncol. 17(8):e328-e346 (2016)]. Bone marrow biopsies and Computed Tomography/Positron Emission Tomography (PET/CT) are utilized to assess response in patients with non-secretory disease [Kumar S, et al. Lancet Oncol. 17(8):e328-e346 (2016)]. Minimal residual disease is evaluated by flow cytometry, in accordance with the Euroflow standards [Theunissen P, et al. Blood. 129(3):347-357. (2017)]. Patients are grouped into “no response” and “response” as follows: “no response”; stable disease (SD)/progressive disease (PD) and “response” ≥VGPR. Analysis of patients' data grouped by PD/SD, VGPR and CR is also provided.

### Primary and Secondary Endpoints

The primary end points of the study are safety and the determination of the maximum tolerated dose (MTD) of HBI0101. Hematological and non-hematological adverse events are graded according to National Cancer Institute Common Terminology Criteria for Adverse Events (NCI CTCAE), version 5.00. Cytokine Release Syndrome (CRS) and Immune effector Cell Associated Neurotoxicity Syndrome (ICANS) adverse events are graded according to the 2019 American Society for Transplantation and Cellular Therapy (ASTCT) criteria [Lee D W, et al., Biol Blood Marrow Transplant. 2019; 25(4):625-638]. Secondary end points included: overall response rate (ORR), and progression-free survival (PFS) and overall survival (OS).

A positive correlation between the level of BCMA expression at the BM-plasma cell surface of MM patients and responsiveness to HBI0101 treatment was established (FIGS. 19A and 19B). In addition, the pharmacokinetics of HBI0101 cells is assessed at serial time-points in the peripheral blood (FIG. 19C). The level of BCMA+ plasma cells in the bone marrow, and the level of circulating soluble BCMA in peripheral blood is also evaluated before and after treatment.

Still further, cytokine/chemokine secretion is evaluated in the blood of subjects before and after infusion of the CART of the invention.

### Example 10

### Patients and Disease Characteristics

Between Jan. 24, 2021, and Dec. 23, 2021, 22 patients were screened, enrolled and leukapheresed. For three patients, apheresis material was cryopreserved; two patients were discontinued (one patient achieved CR, one patient died) before CART cell manufacture (FIG. 13). HBI0101 drug products (DPs) were successfully generated from fresh (n=19) and cryopreserved (n=1) raw materials (no production failure occurred) and infused back to patients.

Three patients received bridging therapy during the manufacturing period. Two patients treated with localized radiotherapy for pain control and one patient received high dose dexamethasone and plasmapheresis due to hyperviscosity. The majority of patients (n=18) underwent lymphodepletion with fludarabine-cyclophosphamide, two patients received bendamustine due to renal impairment (FIG. 13). All the patients were infused with fresh DPs after ten days of production. The median duration of hospitalization post CART infusion was 17 days (range 11-166), with the majority of the patients discharged after a maximum of 25 days, except for P2 who had prolonged neutropenia and thrombocytopenia, and thus remained hospitalized for 166 days.

Patients' characteristics are detailed in Table 2. The median age was 62 years (range, 44-75), with a median time of 55 months from initial disease diagnosis (range, 8-241) and a median number of 6 previous treatment lines (range, 3-13). The majority of patients underwent previous autologous bone marrow (BM) transplantation (85%) and all were refractory to proteasome inhibitor (PI), immunomodulatory agent (IMiD), anti-CD38 antibody (daratumumab) and their last treatment line. Seven of twenty patients (35%) were penta-refractory. Nine patients (45%) were previously exposed and refractory to an anti-BCMA conjugated antibody (belantamab mafodotin) (Table 2). A detailed description of previous treatment lines and refractoriness is presented in Table 1. Patients in cohort 1 had a higher rate of extramedullary disease and penta-refractory disease, high LDH and extensive BM involvement (as defined by >50% plasma cells in BM biopsy), while patients in cohort 3 had a worse ECOG performance score and a lower incidence of cytogenetic high-risk disease (Table 2).

TABLE 2
Patients and disease characteristics

| | Total | Cohort 1 | Cohort 2 | Cohort 3 |
|---|---|---|---|---|
| Variables | N = 20 | n = 6 | n = 7 | n = 7 |
| Age (Years) ^{a} | 62 (44-75) | 58.5 (44-75) | 62 (54-73) | 62 (50-72) |
| Gender ^{b} F/M | 12/8 | 4/2 | 4/3 | 4/3 |
| Time Since Diagnosis (months) ^{a} | 55 (8-241) | 42 (20-123) | 78 (44-241) | 43 (8-104) |
| Extra-medullary Disease (%) ^{b} | 6 (30%) | 3 (50%) | 1(14%) | 2 (29%) |
| LDH above normal (%) ^{b} | 8 (40%) | 3 (50%) | 2 (29%) | 2 (29%) |

ECOG performance status ^{b}

| | | | | |
|---|---|---|---|---|
| 0 | 7 (35%) | 3 (50%) | 2 (29%) | 2 (29%) |
| 1 | 4 (20%) | 1 (17%) | 2 (29%) | 1 (14%) |
| 2 | 9 (45%) | 2 (33%) | 3 (43%) | 4 (57%) |
| Creatinine Clearance | 7 (35%) | 1(17%) | 3 (43%) | 3 (43%) |
| (<60 ml/min) ^{b} | | | | |
| Ejection Fraction (<55%) ^{b} | 3 (15%) | 0 | 2 (29%) | 1 (14%) |

Involved Heavy Chain ^{b}

| | | | | |
|---|---|---|---|---|
| IGG | 4 (20%) | 1 (17%) | 1 (14%) | 2 (29%) |
| IGA | 5 (25%) | 1 (17%) | 2 (29%) | 2 (29%) |

Involved Light Chain ^{b}

| | | | | |
|---|---|---|---|---|
| kappa | 11 (55%) | 2 (33%) | 4 (57%) | 5 (72%) |
| lambda | 8 (40%) | 4 (67%) | 3 (43%) | 1 (14%) |
| Non-Secretory | 1 (5%) | 0 | 0 | 1 (14%) |
| BM Involvement ^{b} | 16 (80%) | 5 (83%) | 6 (86%) | 5 (72%) |
| ≥50% | 6 (30%) | 3 (50%) | 2 (29%) | 1 (14%) |
| PET Positivity ^{b} | 11 (55%) | 4 (67%) | 2 (29%) | 5 (72%) |

R-ISS ^{a}

| | | | | |
|---|---|---|---|---|
| I | 1(5%) | 1(17%) | 0 | 0 |
| II | 11(55%) | 0 | 4(57%) | 7(100%) |
| III | 2(10%) | 2(33%) | 0 | 0 |

Cytogenetic Abnormalities ^{b}

| | | | | |
|---|---|---|---|---|
| High Risk* | 10 (50%) | 4 (67%) | 5 (72%) | 1 (14%) |
| Standard risk | 12 (60%) | 5 (83%) | 5 (72%) | 4 (57%) |
| Unknown | 1 (5%) | 1 (17%) | | |
| Previous Auto HSCT ^{b} | 17 (85%) | 5 (83%) | 7 (100%) | 5 (72%) |
| >1 | 8 (40%) | 2 (33%) | 4 (57%) | 1 (14%) |
| No. of Previous Lines | 6 (3-13) | 5 (4-7) | 9 (3-13) | 5 (3-7) |

^{a} Median (range);
^{b} no. (%)
Abbreviations: ECOG- Eastern Cooperative Oncology Group, R-ISS- Revised International Staging System;
*High-risk cytogenetic abnormalities included: del(17p), t(4; 14), and t(14; 16).

### Example 11

### HBI0101 CART Cells Manufacture

HBI0101 CART cells were locally produced at the good manufacturing procedure (GMP)-accredited facility for advanced cellular therapy, at Hadassah Medical Center, with successful manufacture for all the patients. The median lymphocyte count at the time of apheresis collection was 1.0×10⁶/mL (range, 0.5-2.1), with no impact on CART cell successful manufacture. All final DPs were released in compliance with the criteria specified in Table 3. No significant differences in the production data or in vitro functionality of CART cells of the “response” and the “no response” groups (Table 3) were observed, attesting to the robustness of the production process, despite the high variability in the starting materials of MM patients.

TABLE 3
HBI0101 final product characteristics and release criteria

| | | |
|---|---|---|
| No Response | Response | Release criteria |

DP Identity

| | | | |
|---|---|---|---|
| % Viability | 94.2 ± 1.3 | 96.2 ± 0.3 | ≥70.0% |
| CAR+CD3+ cells (%) | 99.7 ± 0.0 | 99.6 ± 0.1 | ≥70.0% |
| CAR+CD3+CD4+ cells (%) | 31.6 ± 9.1 | 16.7 ± 3.1 | ND* |
| CAR+CD3+CD8+ cells (%) | 66.6 ± 8.5 | 81.4 ± 2.9 | ND* |

DP Potency

| | | | |
|---|---|---|---|
| CAR+ cells (%) | 47.8 ± 2.7 | 52.8 ± 2.8 | ≥10.0% |
| CAR+CD3+CD4+CD56+ | 11.4 ± 5.3 | 19.3 ± 4.3 | ND* |
| cells (%) | | | |
| CAR+CD3+CD8+CD56+ | 39.0 ± 10.8 | 51.8 ± 4.6 | ND* |
| cells (%) | | | |
| IFN-γsecretion (fg/CAR + | 246.2 ± 54.1 | 336.1 ± 49.9 | 2.0-1000.0 fg**/ |
| cell) | | | CAR+ cell |
| CPN/Td cell | 2.4 ± 0.2 | 2.9 ± 0.2 | ND* |

DP Impurity

| | | | |
|---|---|---|---|
| CD19 (%) | 0.0 ± 0.0 | 0.0 ± 0.0 | ≤1.00% |
| CD14 (%) | 0.0 ± 0.0 | 0.0 ± 0.0 | ≤1.00% |
| CD56 + CD3−(%) | 0.5 ± 0.2 | 0.8 ± 0.1 | ND* |

The production process lasts 10 days for all the patients. HBI0101 drug products (DPs) at day of infusion (day 0) was characterized as follows: DP Identity. The percent of CD3+, CD4+ and CD8+ cells (gated on live CAR+ cells) was determined by flow cytometry. DP Potency. The percent of BCMA.CAR+ cells (gated on live cells) was determined by flow cytometry using human recombinant BCMA protein. The percent of activated cytotoxic T cells (CD3+CD56+) was determined by flow cytometry (gated either on CD4+ or CD8+ T cells). The secretion of TIFN-γ by each H1130101 transduced cell, was assessed by ELISA at day −2. IFN-γ was released into the co-culture supernatant of HBI0101-transduced cells with NCI-H929 (1:1 E:T ratio) following an overnight incubation. The total amount of IFN-γ in each culture well was quantified and then divided by the number of transduced cells introduced into the well. The copy number (CPN) of HBI30101 inserts into every CAR transduced cell was quantified by qRT-PCR as described in Materials and Methods and further adjusted to transduced cell. Release criteria specifications are described in the right column of the table. No statistical significance was observed in none of the released parameters, attesting to the robustness of the production process. DP Impurity. The percent of “cell impurities”, as to CD19+, CD14+ and CD3-CD56+ (gated on live cells) was determined by flow cytometry, and do not exceed 10% each in all twenty batches. *ND, not determined. **fg, femtogram.

### Example 12

### Clinical Study—Safety Evaluation

### Hematological Toxicities

All patients developed a grade 3-4 neutropenia, two thirds of whom had grade 3 febrile neutropenia (FN) (Table 4). The entire cohort developed grade 3-4 lymphopenia, with ˜60% developing grade 3-4 thrombocytopenia and anemia. There was a higher incidence of thrombocytopenia and FN in cohort 3 compared to cohorts 1 and 2. The median duration of grade 3-4 neutropenia and grade 3-4 thrombocytopenia were 15 days (range, 1-65) and 24 days (range, 8-138), respectively (excluding 3 patients which progressed and died without counts recovery). Less than a third of patients had grade 3-4 cytopenia persisting for more than 28 days after CART cell infusion (Table 4)

TABLE 4
Adverse events and CART-related toxicity

| | | COHORT | COHORT | COHORT |
|---|---|---|---|---|
| TOTAL | | 1 | 2 | 3 |
| ALL | TOTAL | N = 6 | N = 7 | N = 7 |
| GRADES | GRADE | GRADE | GRADE | GRADE |
| N = 20 | 3-4 | 3-4 | 3-4 | 3-4 |

ADVERSE EVENTS

| | | | | | |
|---|---|---|---|---|---|
| Hematologic ≤28 d | | | | | |
| Neutropenia | 20(100%) | 20(100%) | 6(100%) | 7(100%) | 7(100%) |
| Thrombocytopenia | 14(70%) | 12(60%) | 3(50%) | 4(57%) | 5(71%) |
| Anemia | 19(95%) | 13(65%) | 3(50%) | 6(86%) | 4(57%) |
| Lymphopenia | 20(100%) | 20(100%) | 6(100%) | 7(100%) | 7(100%) |
| Febrile Neutropenia | 13(75%) | 13(75%) | 3(50%) | 4(57%) | 6(86%) |
| Hematologic > 28 d | | | | | |
| Neutropenia | 12(60%) | 6(30%) | 1(17%) | 2(29%) | 3(43%) |
| Thrombocytopenia | 15(75%) | 7(35%) | 2(34%) | 2(29%) | 3(43%) |
| Anemia | 14(70%) | 0 | 0 | 0 | 0 |
| Lymphopenia | 15(75%) | 6(30%) | 3(50%) | 1(14%) | 2(29%) |
| Hypogammaglobulinemia | 14(70%) | 5(25%) | 2(34%) | 1(14%) | 2(29%) |

Other

| | | | | | |
|---|---|---|---|---|---|
| ≤28 d | | | | | |
| Renal Failure | 3(15%) | 0 | 0 | 0 | 0 |
| Elevated Liver Enzymes | 2(10%) | 2(10%) | 1(17%) | 0 | 0 |
| Sepsis | 3(15%) | 3(15%) | 0 | 2(28%) | 1(14%) |
| Infectious Gastroenteritis | 1(5%) | 1(5%) | 0 | 1(14%) | 0 |
| Atrial Fibrillation | 1(5%) | 0 | 0 | 0 | 0 |
| Pulmonary Edema | 1(5%) | 1(5%) | 0 | 1(14%) | 0 |
| Urinary Tract Infection | 1(5%) | 0 | 0 | 0 | 0 |
| Deep Vein Thrombosis | 2(10%) | 0 | 0 | 0 | 0 |
| >28 d | | | | | |
| Atrial Fibrillation | 1(5%) | 1(5%) | 1(17%) | 0 | 0 |
| Pulmonary Edema | 1(5%) | 1(5%) | 1(17%) | 0 | 0 |
| Elevated Liver Enzymes | 1(5%) | 1(5%) | 0 | 0 | (14%)1 |
| Pneumonia | 1(5%) | 0 | 0 | 0 | 0 |
| Pulmonary Embolism | 1(5%) | 1(5%) | 1(17%) | 0 | 0 |

Abbreviations: CRS—Cytokine release syndrome, ICANS—Immune cell associated Neurotoxicity

### CRS and ICANS

Ninety percent of patients developed any grade cytokine release syndrome (CRS), mostly at the day of CART infusion at a median of 0 (range, 0-21), with a median duration of two days (range, 1-5), and none developed grade 3 or more CRS (Table 5). However, a higher rate of CRS grade 2 was noted in cohorts 3 and 2 as compared with cohort 1 (3/7, 4/7, 1/6, respectively) (Table 5). Tocilizumab was used in 40%) percent of patients with a median of one dose administered 1 (range, 1-4). There was no event of immune effect or cell-associated neurotoxicity syndrome (ICANs), and none required glucocorticoids.

TABLE 5
CAR T associated CRS and ICANS

| | | | |
|---|---|---|---|
| All (N = 20) | Cohort 1 (n = 6) | Cohort 2 (n = 7) | Cohort 3 (n = 7) |

CRS (Grade 0-2)

| | | | | |
|---|---|---|---|---|
| Start day ^{a} | 1 (0-21) | 6 (0-21) | 0 (0-1) | 1 (0-3) |
| Duration days ^{a} | 2 (1-5) | 3 (0-5) | 2 (1-3) | 2 (1-5) |
| Tocilizumab given ^{b} | 8 (40%) | 1 (17%) | 4 (57%) | 3 (43%) |
| Grade 2 events ^{b} | 18 (90%) | 1 (17%) | 4 (57%) | 3 (43%) |

ICANS (Any Grade)

| | | | | |
|---|---|---|---|---|
| Events | 0 | 0 | 0 | 0 |

^{a} Median (range);
^{b} number of patients (range).

### Non-Hematological Toxicities

Non-hematological toxicities (Table 4) were reported in 80% (n=16) of patients. Bacteremia was documented in 15% of patients. Grade 3-4 reported toxicities were sepsis (n=3), elevated liver enzymes (n=2), atrial fibrillation (n=1), infectious gastroenteritis (n=1), pulmonary edema which was cardiac secondary to fluid overload and sepsis (n=2), all of which resolved. One patient developed grade 3 pulmonary embolism (PE) while in remission, 10 months after CART therapy. PE developed due to patient's immobilization following infection with pseudomonas pneumonia, which was successfully treated with anticoagulation. Five patients were re-hospitalized during the subsequent follow-up period, due to COVID19 infection for observation (n=1), 9 months after infusion of cells; pulmonary embolism, pneumonia and atrial fibrillation, treated with anticoagulation, antibiotics and cardioversion, 8 months after infusion of cells (n=1); infectious gastroenteritis due to salmonella and adenovirus in stool treated with antibiotics and fluids (n=1), 27 days after infusion of cells; fever treated with broad spectrum antibiotics and 2 doses of tocilizumab (n=1), 21 days after infusion of cells; nausea, vomiting and dyspnea resolved with fluids and antiemetics (n=1), 19 days after infusion of cells (Table 4). No other severe adverse effects (SAEs) were observed in any of the cohorts. One patient died within 30 days of infusion due to disease progression. There were no treatment-related mortalities.

### Example 13

### Short-Term Efficacy

At a median follow-up of 136 days (80, 182 and 160 for cohort 1, 2 and 3, respectively), ORR was 75% for the entire cohort with 50% (3/6), 85% (6/7) and 85% (6/7) responding patients in cohorts 1, 2 and 3, respectively (FIG. 14A). Ten patients achieved (stringent) complete response (sCR/CR), with six patients achieving minimal residual disease (MRD) negativity (four in cohort 3); five patients achieved very good partial response (VGPR) (four in cohort 2), with two patients achieving MRD negativity (FIG. 14A-14B). All patients' best responses were achieved one month post CART infusion, except patients P7 and P11 who achieved VGPR at their first follow-up, and further deepened their response to sCR/CR and VGPR MRD—few months later, respectively (FIG. 14B). The median PFS for the entire cohort was 160 days (range, 14-326+); 80 days (range, 23-248), 182 days (range, 33-326+) and not reached (range, 14-223+) for cohort 1, 2 and 3, respectively (FIG. 15A-B). The median OS was 308 days (range, 25-466+), with an estimated OS of 55% as of data cutoff June 27^{th} the median follow-up for all three cohorts was 160 days (range, 14-326). The median OS was 237 days (range, 25-466+), 282 days (range, 63-347+), and not reached in cohort 1, 2 and 3, respectively (FIG. 15D). All deaths (9/20, 45%) but one, were attributed to disease progression. P7's death was related to COVID19 infection. FLC levels prior to and following CART cell infusion in responders vs. non-responders are shown in FIG. 16A-C (F3A-C#2). In addition, soluble BCMA (sBCMA) levels, as a biomarker of responsiveness to anti-myeloma therapies 9,10,17-19, declined rapidly in the serum of the responding, while its level was barely affected in the non-responding patients (FIG. 16D). In addition, median LDH levels prelymphodepletion tended to be higher in the non-responding patients compared to those who responded (p=0.068). CRP peak level, fibrinogen levels and the relative increase in ferritin levels during the first 14 days following CART infusion were not found to correlate with response (FIG. 17A-17D). No significant difference in terms of CRS was observed between the “response” vs. “no response” groups (data not shown).

### Example 14

### Evaluation and Phenotypic Characterization of BM-Plasma Cells (PCs)

In order to assess patient's response to HBI0101-treatment at the cellular level, BM aspirates were analyzed by flow cytometry prior to (n=12) and one month following CART-administration (n=11) (FIG. 18A). Of these patients, three patients were nonresponsive to HBI0101-therapy, and showed only a minor decline or even an increase in the percent of BM-PCs (FIG. 18B). In contrast, nine patients who did respond showed a significant reduction in the percent of BM-PCs (FIG. 18B). In addition, we found that the baseline BCMA percent expression and mean fluorescence intensity (MFI) on the surface of PCs was significantly higher in patients in the “response” group, compared to patients in the “no response” group (FIG. 18C-18D and FIG. 19A-19B). Interestingly, it was found that the CD56 molecule was significantly expressed on the PC of HBI0101-responding patients (FIG. 18E).

### Example 15

### CART Cell Kinetics

The pharmacokinetics of HBI0101 cells was assessed at serial time-points in the peripheral blood of MM patients following CART administration. The median time to HBI0101 peak concentration (Cmax) was day 10 (range, 6-13) in the “response” group (sCR/CR (range, 6-13) and VGPR (range, 10-13)), and day 13 (range, 10-13) in the “no response” group (FIG. 20D, and FIG. 19F), with a rapid decline in HBI0101 cells proliferation within a month post CART infusion. The area under the curve (AUC), as measure of overall CART cell expansion within the first month post CART infusion, was at borderline significance (p=0.0597) between the two groups (FIG. 20B). This difference was more pronounced when patients were classified into SD/PD, VGPR and sCR/CR subgroups (FIG. 19C-19D). In line with this observation, Cmax values, as a measure of maximal CART-cell expansion, were found significantly different between the two groups (60,655 HBI0101 cells/mL blood (range, 4,945-493,152) in the “response” group vs. 3,740 HBI0101 cells/mL blood (range, 1,117-21,857) in the “no response” group) (FIG. 20C). FIG. 19E further indicates that increased Cmax values correlates with depth of response to HBI0101. It is noteworthy that, while a significant difference in the levels of sBCMA was observed between cohort 1 and cohorts 2 and 3 (p<0.0004), no significant difference in terms of HBI0101 cell kinetics in the peripheral blood was found between these cohorts (FIG. 21). In the “response” group, the decline observed in sBCMA levels is concomitant with HBI0101 CART expansion in the peripheral blood in contrast to the “no response” group (FIG. 20E-20F), suggesting that sBCMA decrease is associated with HBI0101 CART cells anti-myeloma activity, which results in PCs eradication, and subsequent sBCMA clearance.

### Example 16

### Cytokine Profiling Analysis to Predict Response to HBI0101 Therapy

To better understand and potentially predict patients' responsiveness to HBI0101-therapy, the cytokine “signature’ which can be associated with the HBI0101 CART cells' anti-myeloma function in vivo, was next determined. To this end, sera were collected from MM patients at baseline (day −10) and at Tmax (day of Cmax of each patient) and

analyzed for cytokines secretion by multiplex array. The multiplex panel included 25 pro- and anti-inflammatory chemokines/cytokines. Among the 25 biomarkers analyzed, the cytokines that were differently expressed in the different groups (at baseline and Tmax) are described in the heat-map representation (FIG. 22A). Of these, six cytokines, IL-1ra, IL-10, CCL4, IL-15, G-CSF, and IL-6 were significantly differentially expressed between the different groups (FIG. 22B(i)-22B(vi), respectively). More specifically, IL-10, CCL4, and IL-15 were found at higher levels in the “no response” group, while G-CSF, and IL-6 were found at higher levels in the “response” group, at Tmax (FIG. 22A and Table 6). IL-1ra was significantly upregulated in the “no response” group but not in the “response” group at Tmax. As shown in FIG. 22A, another cytokine with a significant increase at Tmax in the “response” vs. “no response” patient groups was IFN-γ. No statistical difference was observed in the level of TNF-α or IL-2, in either groups (FIG. 22C (i)-22C(iii)).

TABLE 6
Cytokine profiling of MM patients

| | No Response | | | | Response | | | |
|---|---|---|---|---|---|---|---|---|
| pg/mL | Baseline | | Tₘₐₓ | | Baseline | | Tₘₐₓ | |
| IL-1ra* | 412 | (329-683) | 873 | (306-8624) | 558 | (7-1960) | 907 | (229-2706) |
| CCL2 | 502 | (441-768) | 830 | (424-1796) | 516 | (13-832) | 690 | (423-1608) |
| CXCL10 | 189 | (150-502) | 672 | (301-2234) | 168 | (3-500) | 421 | (190-1490) |
| G-CSF | 21 | (8-502) | 52 | (10-301) | 168 | (5-500) | 421 | (67-1490) |
| IL-10* | 169 | (88-201) | 462 | (193-3864) | 107 | (14-709) | 255 | (101-618) |
| CCL4* | 223 | (186-452) | 347 | (210-713) | 175 | (70-246) | 199 | (95-590) |
| VEGF | 137 | (52-1218) | 211 | (113-1265) | 168 | (4-535) | 166 | (59-871) |
| IL-6* | 16 | (8-21) | 23 | (10-38) | 14 | (1-70) | 92 | (7-758) |
| IL-8 | 21 | (14-37) | 50 | (30-110) | 23 | (1-119) | 70 | (30-165) |
| GM- | 32 | (23-64) | 77 | (44-145) | 22 | (10-61) | 68 | (24-117) |
| CSF | | | | | | | | |
| CCL3 | 34 | (19-148) | 119 | (84-137) | 28 | (10-135) | 47 | (11-774) |
| IL-15* | 5 | (3-7) | 14 | (8-15) | 3 | (1-10) | 6 | (3-13) |
| IFN-γ* | 58 | (31-137) | 87 | (44-121) | 30 | (0-91) | 86 | (20-231) |
| IL-2 | 12 | (0-257) | 0 | (0-80) | 35 | (0-146) | 17 | (0-79) |
| TNF-α | 62 | (15-1463) | 137 | (20-751) | 22 | (7-535) | 105 | (15-468) |

Concentrations are in pg/mL.
*Cytokines that were significantly differently expressed in the different groups (see FIG. 22B-22C).
Baseline cytokine concentration was determined at day −10 prior to CART administration.
Tₘₐₓ, time of concentration of CART cells at peak.

### Example 17

### Effect of Exposure to Previous Anti-BCMA Antibody on HBI0101-Therapy

In this study, 9/20 patients (2/6 from cohort 1, 5/7 from cohort 2 and 2/7 from cohort 3) had received belantamab mafodotin prior to HBI0101-therapy. Patients exposed to belantamab (“belantamab(+)”) tended to have a shorter PFS and higher progression rate (7/9, 78%) in comparison with the non-exposed (“belantamab(−)”) patients (6/11, 55%) (FIG. 23A). Moreover, the OS was lower in “belantamab(+)” patients (3/9, 33%) than the OS of “belantamab(−)” patients (8/11, 73%) (FIG. 23B). ORR was higher in the “belantamab(−)” group in comparison with the “belantamab(+)” group (91% vs. 55%, respectively) (FIG. 23C). Notably, when the inventors looked at “depth of response”, it was found that “belantamab(−)” patients achieved significantly deeper responses than “belantamab(+)” patients (sCR/CR, p=0.002; VGPR, p=0.02; by unpaired t-test). However, there was no significant difference in the levels of BCMA expression on PCs (determined by MFI), and in the percent of BCMA-positive PCs between the two groups. FIGS. 23D and 23E show the expression of BCMA in the patients.

These results represent a full CART product cycle: development of a new CART treatment, proof-of-concept in-vitro and in-vivo, approval by regulatory authorities, local production in a GMP facility, and delivery to patients (FIG. 24A-24B). Major advantages of such an approach over commercial products are the availability of the DP and the shortened “vein-to-vein” delivery. Moreover, the significance of these results extends far beyond the clinical dimension alone. An affordable, locally produced CART represents a major step in broadening access to this cutting edge advanced cellular therapy.

### Example 18

### BCMA Expression in AL- Versus MM-PCs

Primary light chain amyloidosis (AL) is a rare monoclonal plasma cell (PC) disorder characterized by the systemic deposition of misfolded immunoglobulin light chain (LC) protein product, as insoluble fibrils in multiple organs. Since AL is a disease originating in malignant PCs, most available treatments are adopted from multiple myeloma-directed therapies. Yet, due to the wide clinical spectrum of organ failure in AL, the same regimens may be extremely toxic to these patients (1-4), and multiple myeloma-oriented clinical trials usually exclude patients with AL. The present disclosure describes herein the ex vivo applicability anti-BCMA CAR construct of the disclosure on AL primary cells, as well as the safety and efficacy in four patients with relapsed/refractory (RR) primary AL, treated in the phase I clinical trial (NCT04720313) described above in EXAMPLE 9.

As a preclinical evaluation of HBI0101-based CART for the treatment of MM and amyloidosis, the percent of BCMA+ cells in the BM of MM and AL patients was assessed, and the expression levels of BCMA on AL- and MM-PCs was measured by flow cytometry (FIG. 25A(i)). While the BM samples of AL- and MM-patients displayed similar percentage of BCMA+ cells, FIG. 25A(ii) indicates that the mean fluorescence intensity (MFI) of BCMA on AL-PCs was significantly lower (Average MFI=1.9 in AL patients, n=18 vs. MFI=3.8 in MM patients, n=39; p<0.05). However, based on the preclinical data disclosed above for MM clinical study, the inventors showed that MM-PCs with an average MFI of BCMA expression of approximately 2.1 were eradicated following incubation with BCMA. CART cells, and in line with the recent publication on BCMA-targeting in AL amyloidosis patient [Oliver-Caldes A, 9(12) (2021)], it was therefore anticipated that this level would be sufficient for PC recognition by BCMA-CART cells and may represent a potential target for the treatment of AL.

### Example 19

### Evaluation of HBI0101 Efficacy Against Primary AL-PCs Ex-Vivo

The efficacy of the HBI0101 CART against MM-PCs ex-vivo was demonstrated in EXAMPLES 9-17, herein above. Encouraged by the results with MM patients, the inventors next tested whether HBI0101-based CART is efficient in the eradication of AL-primary PCs as well. Following an overnight co-culture with HBI0101, an almost complete eradication of AL-PCs by HBI0101 was observed (FIG. 25B), effect that was already evident after one hour of co-culture (FIG. 26). In contrast, AL-PCs were not affected by the presence of non-transduced (NT) control cells, suggesting that HBI0101 cells were able to recognize AL-PCs and exert specific BCMA-directed antitumoral function. In this regard, non-tumor BM-MNCs were not affected by the presence of HBI0101 as indicated by 7AAD staining (FIG. 26). In addition, following incubation with AL BM-MNCs, HBI0101 cells underwent significant activation evidenced by the upregulation of the 4-1BB marker and increased secreted levels of the pro-inflammatory cytokines IFN-γ and TNF-α (FIG. 25C(i), 25C(ii), 25C(iii), respectively), in comparison with NT cells (p<0.05). The inventors therefore concluded that HBI0101-mediated anti-BCMA functions are restricted to CD38++CD138++ cells, while sparing the other BM cell populations.

To further substantiate the observation that PC elimination by HBI0101 cells is apoptosis-mediated, the intracellular level of cleaved-caspase-3 was analyzed. FIG. 25D confirms that, following incubation with HBI0101 cells, but not with NT cells, there is a marked increase in the apoptosis of AL- or MM-magnetically enriched CD138+BM-PCs (FIG. 27). Moreover, FIG. 28 further suggests that HBI0101 cell activation and proinflammatory cytokine secretion upon stimulation with AL- or MM-CD138+ cells are specifically mediated via BCMA-CAR, since NT cells showed significantly lower levels of 4-1BB (FIG. 28A) and barely detectable levels of IFN-γ and TNF-α (FIG. 28A-28C), regardless of whether NT cells were incubated with CD138+(FIG. 28) or with the CD138—cellular fraction. Data attesting for the specificity of HBI0101 against BCMA-expressing myeloma cells are provided herein above in EXAMPLES 9-17 and is also provided in FIG. 29A-29B, as well. Altogether, these data support the proof-of-concept for the potential efficacy of HBI0101 CAR T-based therapy for the treatment of AL.

### Example 20

### HBI0101 CART Cell Production and Treatment of AL Patients.

In view of the ex-vivo results and toward clinical evaluation of HBI0101 in AL patients, the inventors generated autologous BCMA-CART cells for Patients 1-4 (obtained by leukaphereses procedure). Following a 10-day production, all four patients were infused with fresh BCMA-CART cells. Although CART manufacturing was initiated from diverse apheresis sources, the process was shown robust with final drug products (DPs) meeting release criteria (Table 7). All four clinical batches shared similar expansion pattern in culture (days −7 until day 0) and DPs' characteristics as shown by FIG. 30. FIG. 30C(i)-30C(ii), 30D(i)-30D(ii) depicts the exhaustion and differentiation statuses, respectively, of the DPs at the day of release (day 0).

TABLE 7
Release testing criteria

| | Description | Requirement |
|---|---|---|
| Cell Quantity | HBI0101 cell dose | 150/450/800 × 10\hat{ }6 CAR+ cells ±20% |
| | Total cell dose | 100-8000 × 10\hat{ }6 cells |
| | HBI0101 cell concentration | 10-100 × 10\hat{ }6 CAR+ cells/mL |
| | Total cell concentration | 10-150 × 10\hat{ }6 cells/mL |
| | Drug product volume | 10-100 mL |
| Cell Identity | Cell viability | ≥70.0% |
| | Percent of CD3+ T cells | ≥70.0% |
| Cell Impurity | Percent of CD19+ B cells | ≤1.00% |
| | Percent of CD14+ monocytes cells | ≤1.00% |
| Cell Potency | IFN-γsecretion following co-culture | 2.0-1000.0 fg/transduced cell |
| | with BCMA-expressing target cells | |
| | Percent of transduction | ≥10.0% CAR+ cells |
| Non-cellular | Appearance | Homogenous slightly yellow cell suspension |
| | | without visible clumps or foreign particles |
| characterization | pH | 6.0-8.0 |
| Safety | Determination of Replication | Not detected (<10\hat{ }-1 GaLV copies) |
| | Competent Virus (RCR) by PCR | |
| Sterility | Rapid sterility by Gram stain | No evidence of bacterial cells |
| | Bacterial Endotoxin | ≤1.00 EU / mL |
| | Sterility by Direct Inoculation | No Growth |
| | culture | |
| | Sterility by BactAlert culture | No Growth |

Competent Retrovirus; GaLV: Gibbon Ape Leukemia Virus
fg: femtogram (10-15 gram); PCR: Polymerase Chain Reaction; RCR: replication competent
retrovirus.

### Example 21

Patients with AL Treated with HBI0101

This study aimed at evaluating HBI0101 safety and efficacy in patients with multiple myeloma and additional plasma cell dyscrasias, including Amyloidosis (AL). Patients enrolled had to be refractory to at least three lines of treatment including a proteasome inhibitor, an immuno-modulator (IMiD) and an anti-CD38 antibody, and to have no other available registered therapy. The phase I clinical trial consisted of three escalating-cell doses, as described in EXAMPLE 9. Three patients were treated within this clinical trial (NCT04720313) in a different safety cohort, and the fourth was treated on a compassionate basis. Patients' baseline characteristics, history and previous treatments are summarized in Table 8. All patients were refractory to their last treatment line, three were penta-refractory, and two were anti-BCMA belantamab mefadotin refractory. All patients had a progressive disease at enrollment. None received any bridging therapy. The complete study protocol is further outlined in FIG. 10.

Before patient's enrollment into HBI0101 study, candidates are screened for eligibility. At day −10 prior to infusion, lymphocytes are collected using the Sprectra Optia apheresis instrument. Immediately after, patients are hospitalized for baseline assessment, while HBI0101 production is initiated. Patients are then T-cells depleted on days −5 to −2 with 25 mg/m2 fludarabine and 250 mg/m2 cyclophosphamide, and after two days of “wash-out” from lymphodepletion, infused with the manufactured HBI0101 on day 0. Two weeks of hospitalization post HBI0101 infusion are mandatory for safety follow up. Continued follow up until progression or death is routinely performed.

TABLE 8
Patients' characteristics at enrollment

| | Patient 1 | Patient 2 | Patient 3 | Patient 4 |
|---|---|---|---|---|
| Age | 64 | 58 | 82 | 63 |
| Gender | Male | Female | Male | Male |
| Concomitant MM | Yes | No | No | No |
| Involved FLC (mg/L) | 155 | 183 | 87 | 560 |
| dFLC (mg/L) | 143 | 177 | 50 | 550 |
| BMPCs (%) | 3 | 15 | 1 | 15 |
| FISH cytogenetics | T11: 14 | T14: 16 | 14Q- NOS | T11: 14 |
| | | 1Q+ | | |
| Organ involvement | Cardiac | Cardiac | Renal | Cardiac |
| | Renal | Renal | GI | Hepatic |
| | Autonomic | Hepatic | | Lung |
| | | | | Soft tissue |
| | | | | Autonomic |
| NYHA stage | 3 | 4 | 1 | 3 |
| ProBNP (pg/ml) | 7500 | 2008 | 119 | 2773 |
| Trop T (ng/L) | 60 | 40 | 8 | 78 |
| Creatinine (mmol\L) | 80 | 72 | 110 | 100 |
| Albuminuria (g/24 h) | 0.3 | 0.3 | 2.4 | 0.1 |
| ALKP (u/L) | 45 | 218 | 84 | 140 |
| MAYO stage | 3a | 3a | 1 | 3a |
| ECOG PS | 0 | 2 | 0 | 0 |
| Prior lines of therapy | 8 | 6 | 6 | 10 and MDS |
| Best response/which line | VGPR/3^{rd} | VGPR/2^{nd} | CR/1^{st} | CR/1^{st} and 4^{th} |
| Previous ASCT | Yes | Yes | No | Yes (as salvage) |
| Triple-drug refractory | Yes | Yes | Yes | Yes |
| Penta-drug refractory | Yes | Yes | No | Yes |
| belantamab refractory | No | Yes | No | Yes |
| Last line refractory | Yes | Yes | Yes | Yes |
| Years since diagnosis | 10.5 | 4 | 15 | 4.5 |

FLC: free light chain; BMPC: bone marrow plasma cell content; FISH: fluorescence in situ hybridization; NYHA: New York Heart Association; BNP: brain natriuretric peptide; Trop T: Troponin T (High sensitivity); ALKP: Alkaline phosphatase; PS: performance status; ASCT: autologous stem cell transplantation.

Patient 1: A 61-year-old male with concomitant MM and AL with cardiac, renal and autonomic involvement, previously treated with eight prior lines of therapy. At the timing of CART, clinical NYHA 3 was apparent, and MAYO stage 3A disease, with a proBNP of 7500 μg/ml. In April 2021 the patient was enrolled to the first safety cohort of the trial and was infused with 150×10⁶ HBI0101 cells. The patient did not experience CRS (Table 9 or exacerbation of his heart failure and remained stable until discharge. No further adverse events (AEs) or organ decompensation were noted following HBI0101-therapy. The patient remained hypogammaglobulinemic and after 8.5 months presented with a grade 3 pseudomonas pneumonia requiring hospitalization, later resolved with antibiotic treatment.

Patient 2: A 59-year-old female diagnosed with AL, with cardiac, renal and hepatic involvement in 2017. In July 2021, after six prior treatment lines, the patient was enrolled to the second safety cohort, and infused with 450×10⁶ CART cells. At the time of CART infusion, the patient suffered from NYHA grade 4 heart failure and deteriorating liver function tests, elevated alkaline phosphatase levels, with severe anasarca before the infusion, stabilized with supportive therapy. After infusion, the patient experienced a two days of grade 2 CRS (Table 9), which required a single dose of tocilizumab. Heart failure exacerbation was evident prior and throughout the length of hospitalization, requiring high doses of diuretics, and the patient remained stable until discharge. Following discharge, at day 45, the patient developed liver de-compensation, and ascites. No evidence of hepatic or portal venous occlusion. Decompensation gradually resolved, and a hepatic organ response was noted with alkaline phosphatase reduction and eventual resolution of the ascites. At the same time, the patient developed fever, which proved to be secondary to osteomyelitis of the spine, eventually resolved, requiring prolonged antibiotic treatment.

Patient 3: An 82-year-old male diagnosed with AL FLC K amyloidosis in 2006 with renal (3 gr of albuminuria) and gastrointestinal involvements with no cardiac involvement. After six lines of therapy, in November 2021, the patient was enrolled to the third safety cohort and infused with 800×10⁶ HBI0101 cells. The patient experienced a grade 3 CRS which required 3 doses of tocilizumab (Table 9). No renal failure was noted, and he remained stable until discharge, although a grade 3 infection was documented.

Patient 4: The last patient was treated on a compassionate basis because of a myelodysplastic syndrome and low blood counts. A 61-year-old male diagnosed in 2017 with FLC L cardiac, autonomic, soft tissue, and renal AL. The last assessment prior to CART infusion showed clinical NYHA stage 3, MAYO stage 3A disease with a proBNP of 2773 pg/ml. After obtaining an informed consent (the patient fully aware of the CART treatment risks for both diseases) and the authorization of the ethics committee in December 2021, the patient was infused with 450×10⁶ HBI0101 cells. The patient experienced a short grade 3 CRS (Table 9) which required a single dose of tocilizumab. At the time of CRS, a heart failure exacerbation was noted, but this responded to the tocilizumab and supportive care. No renal failure was noted, and after 24 h, Patient 4 remained stable until discharge. Before treatment, the patient's blood counts presented with grade 1 neutropenia, grade 2 anemia and grade 4 thrombocytopenia. These worsened to grade 4 neutropenia, grade 3 anemia and grade 4 thrombocytopenia following HBI0101 infusion. While the neutropenia and anemia recovered to grade 2, grade 4 thrombocytopenia is still ongoing. A repeated biopsy of bone marrow after 30 days of treatment showed severe hypocellularity, which implicates CART toxicity more than a worsening of the MDS.

None of the patients had developed immune effector-cells neurotoxicities (ICANs) or any new treatment-related AEs either, although one early infection (osteomyelitis) at month 2 and one late pneumonia at month 8 post CART therapy were observed. All AEs were short and manageable. All patients show pan-hypogammaglobulinemia and received IVIG supplementations. It should be emphasized that, although an initial cardiac deterioration was noted during and after the CART for Patients 2 (before and after infusion) and 4, it was clinically manageable. Currently, at a median follow up of 5.2 months (2.5-9.5) all patients are alive. The detailed AEs and CART-related toxicity is summarized in Table 9.

### Patient Outcome

All four patients have achieved a hematologic CR with a dFLC of 0-8 mg/L as shown in Table 10 and FIG. 31A. Moreover, uninvolved FLC values were also reduced to below the normal range in comparison of the corresponding values prior to CART treatment (FIG. 31B]. The decrease of both involved and uninvolved FLC 30 days post HBI0101 infusion, reflects the robust effect of HBI0101 on both malignant and normal PCs. Approximately 30 days post HBI0101-treatment, BM MRD assessment at 10⁵ negativity was achieved for patients 1, 3 and 4. While Patient 2 was unavailable for MRD testing at that time, MRD assessed at day 180 post CART infusion showed MRD negativity. The time to best response ranged from 17 to 57 days. Respectively, no patient yet progressed, and the duration of response is ongoing for all patients, albeit the various HBI0101 infusion doses. Although patients' follow-up is short, organ responses were already apparent in all four patients. All cardiac patients showed significant reduction of proBNP levels and clinical improvement of their NYHA status (Table 10). Although Patient 2 initially experienced de-compensation of hepatic function ongoing for three months and cardiac dysfunction, she had recovered over time, and displayed both hepatic and cardiac organ response at six months as manifested by NYHA clinical stage improvement, and resolution of ascites. In fact, all cardiac patients NYHA clinical stage improved and patient 3 with renal involvement had improvement of peripheral edema in parallel to the reduction of albuminuria. Although quality of life (QoL) questionnaires was not performed in this trial, it is evident that clinical amelioration of symptoms was evident and correlated with a subjective QoL improvement. All four patients developed pan-hypogammaglobulinemia following CART infusion (FIG. 31C) and received IVIG supplementation. FIG. 31D shows the expression level of BCMA on AL patients' BMPCs prior to HBI0101 infusion, while FIGS. 31E-31F show the decrease in the percent of BM PCs at 30-days post HBI0101 infusion. In addition, PET-CT scan of Patient 1 with concomitant M 30-days post HBI0101 infusion, shows a marked decrease in size and intensity at most tumor sites in comparison with the lesions which were detected by PET-CT before CART infusion (FIG. 31G). The detailed efficacy results are summarized in Table 10.

TABLE 9
Adverse events and CART-related toxicity

| | Patient 1 | Patient 2 | Patient 3 | Patient 4 |
|---|---|---|---|---|
| CAR+ cells infused (×10⁶) | 150 | 450 | 800 | 450 |

Adverse events of interest

| | | | | |
|---|---|---|---|---|
| CRS | No | Yes | Yes | Yes |
| CRS grade | | 2 | 3 | 3 |
| Time to onset (days) | | 2 | 3 | 1 |
| CRS duration (days) | | 2 | 4 | 1 |
| Tocilizumab use (number | 0 | 1 | 3 | 1 |
| of doses) | | | | |
| Steroids use | No | No | Yes | No |
| Vasopressor use | No | No | Yes | No |
| High flow oxygen use | No | No | Yes | Yes |
| ICANs | No | No | No | No |

Hematologic AEs (grades)

| | | | | |
|---|---|---|---|---|
| Neutropenia | 1 | 3 | 3 | 4 |
| Anemia | 0 | 1 | 2 | 3 |
| Thrombocytopenia | 0 | 2 | 1 | 4* |
| Duration of Hematologic | 4 | 3 | 5 | Ongoing |
| AE** (days) | | | | >31 |
| AL Organ deterioration | No | Yes | No | Yes |
| CHF exacerbation | No | Yes | No | Yes |
| Acute renal failure | No | No | No | No |
| Hepatic dysfunction | No | Yes | No | No |
| | | Grade 3 | | |

Non-hematologic other AEs (grades)

| | | | | |
|---|---|---|---|---|
| Fatigue | 1 | 2 | 3 | 1 |
| GI | 0 | 0 | 2 | 0 |
| Rash | 0 | 0 | 0 | 0 |
| Febrile Neutropenia | 0 | 0 | 1 | 3 |
| Infections | 0 | 3 | 3 | 3 |
| Hypogammaglobulinemia | Yes | Yes | Yes | Yes |
| Post CART infections | Yes. | Yes. | No | No |
| during follow up | At day 248 | At day 33 | | |
| | pneumonia | osteomyelitis | | |
| | grade 3 | grade 3 | | |

*Started with grade 4 due to MDS.
**Duration to resolution to grade 2 or better.
CRS: cytokine release syndrome; ICANS: immune effector-cells neurotoxicities; CHF: congestive heart failure; GI: gastro-intestinal.

TABLE 10
Efficacy results

| | Patient 1 | Patient 2 | Patient 3 | Patient 4 |
|---|---|---|---|---|
| CAR+ cells infused (×10⁶) | 150 | 450 | 800 | 450 |
| Best hematologic response | CR | CR | CR | CR |
| iFLC at best response (mg/L) | 0.6 | 0.9 | 1 | 7 |
| dFLC at best response (mg/L) | 0 | 0 | 0 | 1.4 |
| MRD (10⁻⁵) negativity at Day | | | | |
| 30 | Yes | | Yes | Yes |
| Day 180 | Yes | Yes | | |
| Time to best confirmed | 27 | 57 | 17 | 17 |
| response (days) | | | | |
| Follow up (months) | 9.5 | 6.5 | 3.5 | 2.5 |
| DOR | 8.5 | 4.4 | 3 | 2 |
| Organ response | Yes | Yes | Yes | Yes |
| Delta response (% reduction) | −4800 | −1295 | NA | −1872 |
| proBNP (pg/ml)/ | (−64%) | (−64%) | | (−68%) |
| Albuminuria (g/d) | NA | NA | −1.03 | NA |
| | | | (−67%) | |
| NYHA change | III to II | IV to II | NA | III to II |
| Additional organ responses | NA | Hepatic: 280 | NA | NA |
| Alk Phos (u/l) | | to 150 | | |

iFLC: involved free light chain; dFLC: delta free light chain; MRD: minimal residual disease; DOR: duration of response; BNP: brain natriuretric peptide.
NYHA: New York heart association.

### Example 22

### HBI0101 CART In-Vivo Kinetics

HBI0101 cell in-vivo expansion was noted in all four infused patients (FIG. 32A). The median time of detectable CAR by qPCR in the peripheral blood was 24±10 days post CART infusion, and T cells were still detectable in all four patients at day 27±10. The peak in the rate of CART cell growth in peripheral blood was observed between days 6-10 post CART infusion (FIG. 32B). Despite receiving low and intermediate doses of CART cells (150− and 450×106), Patients 1 and 4 showed maximal CART cells expansion in-vivo (FIG. 32A). In contrast, Patients 2 and 3 who were infused with intermediate and high doses of CART cells (450− and 800×106, respectively), showed lower in-vivo expansion (FIG. 32A). In line with this observation, higher numbers of CART cells were detected in the bone marrow of Patients 1 and 4 one month following CART cells infusion, while CART cells were barely detected in Patients 2 and 3 (FIG. 32C). Additionally, analyzing the kinetics of the dFLC levels in the serum of all four AL patients prior to, and following CART cell infusion, a significant decline was observed during the first month of treatment. This decrease in dFLC levels was concomitant with CART expansion in the peripheral blood (FIG. 32D). A similar trend was observed measuring the levels of serum BCMA [sBCMA], an additional biomarker for MM and AL monitoring [Ali S A, et al. Blood. 128:1688-700. (2016), Munshi N C, et al. N Engl J Med; 384:705-16., Raje N, et al. NEngl J Med 2019; 380:1726-37. (2019)](FIG. 33). Overall, these data indicate that dFLC and sBCMA serum level reductions correlate with the successful HBI0101 in-vivo expansion.

### Example 23

### Follow-Up of Clinical Trial NCT04720313

An additional follow-up of phase Ia/b study (NCT04720313) was performed by the inventors, for exploring the safety and efficacy effects in 47 patients (42 MM and 5 AL-patients) at escalating doses ranging from 150- to 800×10⁶ CAR+ cells. The median follow-up (mFU) was 146 days (range, 18-314), with a median progression-free survival (mPFS) and overall survival (mOS) not reached yet at the higher dose of 800×10\hat{ }6 CAR+| cells. The overall response rate for all cohorts was 83%, with a 90% response rate in the 800×10⁶ cohort (FIG. 34A). Both OS % (FIG. 34B) and PFS % (FIG. 34C) were significantly better in the 800×10⁶ cohort in comparison with the other previously reported prior art cohorts.

No immune effector cell-associated neurotoxicity syndrome (ICANS) events were recorded. Whileany grade CRS occurred in 90% (38/42) of MM patients and 80% (4/5) of AL patients they were clinically manageable. The vast majority of the CRS events started at the day of infusion. The median duration of the CRS was 2 days (range 1-7) for MM and 4 days (range 1-5) for AL patients. A median of one dose Tocilisumab (range 1-4) was required in ≥Grade 2 CRS events. 2/42 MM and 1/5 AL patients required steroids while 1/42 MM and 3/5 AL patients required Vasopressors. Grade 3-4 of thrombocytopenia occurred in 23/42 MM patients, of anemia in 25/42, of neutropenia & lymphopenia in 42/42 and of febrile neutropenia in 27/42 MM patients. Hypoglobulinemia persisting >28 days occurred in 30/42 MM patients and in 3/5 AL patients. Taken together, the 800×10⁶ dose showed a significantly improved outcome, specifically, an increased efficacy, while not affecting the toxicity. It is noteworthy that owing to the clinically demonstrated safe profile, higher doses of HBI0101 are tolerable, and thereby account for the substantial improved efficacy of HBI0101. In the light of the data presented here, it is clear that H8BB CAR T-cell based therapy provides significant value for the treatment of MM and additional plasma cell-related pathologies.

### DETAILED DESCRIPTION OF THE INVENTION

CAR T-cell strategies are revolutionizing the treatment of CD19⁺ lymphoma. The application of such approaches to other hematological malignancies such as multiple myeloma requires the identification and design of suitable chimeric receptors targeting specific antigens. Herein, the invention has designed and evaluated the therapeutic function of four different BCMA-specific CARs based on the same antibody chains forming the targeting moiety. Two of the assessed CARs incorporated a CD28 moiety (IC28 and H828) while the other two (ICBB and H8BB) were 4-1BB-based. Initial testing indicated the IC28 CAR was the least functional (FIG. 1B), prompting the inventors to focus on the other constructs.

Compared to the evaluated CAR construct herein, H8BB CAR demonstrated in most cases the highest biological activity by means of cytokine secretion, cytotoxicity, and upregulation of activation. Puzzlingly, while both the H8BB of the present disclosure and H828 constructs mediated near complete regression of human tumors in xenograft experiments, the ICBB construct did not display significant biological activity. Several reasons could account for that—indeed, both H8BB and H828 construct comprise hinge and transmembrane region derived from CD8 while the ICBB construct uses a shorter hinge (21 aa, vs 46aa) and the TM from the CD28 molecule. While not much is known about the role of the TM region in CAR function, several studies show that there is a need to custom tailor the hinge nature and length to the targeted antigen [Guedan S, et al., Mol. Ther. Methods. Clin. Dev. (2018); 12:145-156; Lindner S E, et al. Sci. Adv. (2020]. ICBB-CAR lack of in vivo function might be related also to tonic signaling and non-specific activity observed in several assays (as seen for example in cytokine secretion assays or activation marker upregulation with an antigen negative target—FIG. 2A and FIG. 3D respectively, which could eventually lead to hypofunction [Long A H, et al., Nat. Med. (2015); 21:581-590]. When comparing H8BB of the present disclosure, with the prior art H828 construct the present disclosure clearly shows that the former generally demonstrated a superior activity with lower PD1 levels. Both performed effectively in vivo with a significant advantage to the H8BB-CAR group over the H828-CAR group (88% vs 55% survival respectively, log-rank test, p=0.09). Thus, this seems to demonstrate, at least partially, the long-term advantage manifested 4-1BB-based CAR over CD28-based receptors, as previously described [12]. Additionally, as MM is known for its high relapse rates and duration of responses even to novel or immunologic agents, these findings may bear important implications for the clinical implementation of this line of treatment.

Thus, this work strengthens the need to evaluate empirically CARs in order to determine their optimal configuration and presents an optimized BCMA-CAR that demonstrated long term in vivo efficacy. Moreover, by enhancing specificity with reduced “off-target” effects, such protection may be influenced by the nature of the receptor utilized—either its basic structure as shown herein or its origin (e.g., murine vs. human) [Wang D, et al. (2021); 137:2890-2901]. Importantly, considering that BCMA is expressed by plasma cells from the bone marrow of patients suffering from different plasma cells pathologies (FIG. 7A), anti-BCMA CAR T cell therapy may be used for the treatment of more fragile patients with other than MM-plasma cells pathologies, such as light chain amyloidosis. Considering the very encouraging data collected so far as to the safety and efficacy of H8BB in vitro and ex vivo, the inventors aim to assess the therapeutic potential of an H8BB CAR T cell-based treatment in patients with plasma cell pathologies. An academic-based, GMP-grade, H8BB CAR-engineered T cell-based treatment for R/R MM, which was approved by the Israeli Ministry of Health was successfully generated. A clinical study was initiated as disclosed by Example 9, enrolling R/R malignant plasma cell patients to be treated with autologous H8BB-CAR T-cells (NCT04720313) in a dose-escalation study evaluating first the safety and then the efficacy of this approach. In the light of the data presented here, it is clear that H8BB CAR T-cell based therapy as disclosed herein, provides significant value for the treatment of MM and additional plasma cell-related pathologies.

Altogether these data demonstrate that H8BB-based CAR T cells of the present disclosure respond more specifically to stimulation with cognate antigen than ICBB-CART cells, which show “off-target” activity, or H828-CAR T cells, which were found continuously activated regardless of antigen stimulation. These data correlate with the fact that H828 CAR T cells display a significantly more pronounced exhaustion profile than the H8BB CAR T disclosed herein (FIG. 4A). Additionally, H8BB CAR T cells exhibit a reduced tonic signaling in comparison with ICBB CAR T cells, which importantly may impact the “off-target” activity specificity of the cells. Taken together, the present disclosure provides improved CAR molecules and CAR T cells that display superior therapeutic effect.

Thus, a first aspect of the present disclosure relates to a chimeric antigen receptor (CAR) molecule comprising the following components: (i) at least one target-binding domain; wherein at least one of the target binding domain specifically recognizes and binds B cell maturation antigen (BCMA); (ii) at least one hinge and transmembrane domain derived from the Cluster of Differentiation 8 α (CD8α) protein (also known as T-Cell Surface Glycoprotein CD8 Alpha Chain). It should be noted that the hinge region of the domain comprises the amino acid sequence as denoted by SEQ ID NO:9, and any fragments, derivatives and variants thereof. The CAR molecule further comprises (iii) at least one intracellular T cell signal transduction domain. More specifically, this domain comprises at least one domain of tumor necrosis factor (TNF) receptor family member, and optionally, at least one domain of a T cell receptor (TCR) molecule.

The present disclosure thus provides a CAR molecule. Chimeric Antigen Receptor (CAR), as used herein, refers to a recombinant polypeptide comprising at least an extracellular antigen binding domain, a transmembrane domain and an intracellular cytoplasmic signaling domain comprising a functional stimulatory domain. The receptors are chimeric because they couple between extracellular antigen-binding capabilities and intracellular T- or B-cell activating functions, in a single receptor molecule. CARs have been engineered to give the B or T cells they are expressed in the new ability to recognize a specific antigen of interest, thereby facilitating an immune reaction against it. For example, the technology is used in immunotherapy for specifically recognizing specific cancer cells' antigens of interest in order to more effectively direct the immune cells towards those target cells and destroy them.

CAR, as used herein, relates to artificial T cell receptors (also known as chimeric T cell receptors, chimeric immuno-receptors). These are engineered receptors, which graft an arbitrary specificity onto an immune effector cell. Typically, these receptors are used to graft the specificity of a monoclonal antibody onto a T cell.

The initial design (also referred to a fist generation) joined an antibody-derived scFv to the CD3ζ intracellular signaling domain of the T-cell receptor through hinge and transmembrane domains.

Second generation CARs added intracellular signaling domains from various costimulatory protein receptors (e.g., CD28, 41BB, ICOS) to the cytoplasmic tail of the CAR to provide additional signals to the T cell. More recent, third generation CARs combine multiple signaling domains, such as CD27, CD28, 4-1BB, ICOS, or OX40, to augment potency.

It should be understood that the disclosed CAR molecules may be further improved by adding at least one additional signaling domain.

The term “chimeric protein” relates to proteins created through the joining/fusing of two or more genes that originally coded for separate proteins. Translation of this chimeric/fusion gene results in a single or multiple polypeptides with functional properties derived from each of the original proteins. Recombinant chimeric/fusion proteins are created artificially by recombinant DNA technology. Chimeric or chimera usually designate hybrid proteins made of polypeptides having different functions, sources or physico-chemical patterns.

As indicated above, the CAR T molecules disclosed herein are specifically directed against that BCMA protein. B-cell maturation antigen (BCMA), also referred to as TNFRSF17 or CD269, is a member of the tumor necrosis factor receptor (TNFR) superfamily. Ligands for BCMA include B-cell activating factor (BAFF) and a proliferation-inducing ligand (APRIL), of which APRIL has a higher affinity for BCMA. BCMA is expressed preferentially by mature B lymphocytes, with minimal expression in hematopoietic stem cells or nonhematopoietic tissue and is essential for the survival of long-lived bone marrow plasma cells (PCs), but not overall B-cell homeostasis. Membrane-bound BCMA can undergo 7-secretase-mediated shedding from the cell surface, leading to circulation of soluble BCMA (sBCMA) and reduced activation of surface BCMA by APRIL and BAFF. The overexpression and activation of BCMA are associated with MM. Moreover, the use of BCMA as a biomarker for MM is supported by its prognostic value. Still further, in some embodiments, BCMA as used herein refers to the human BCMA, that comprises the amino acid sequence as denoted by Q02223. In yet some further embodiments, BCMA is encoded by the nucleic acid sequence as denoted by Genebank accession Number Z14954.1. In yet some further embodiments, BCMA comprises the amino acid sequence as denoted by SEQ ID NO: 25, and any variants or derivatives thereof.

In some embodiments, the at least one target binding domain of the CAR-molecule of the present disclosure comprises: (i) at least one target-recognition element; and/or (ii) at least one adaptor component that recognizes and binds at least one tagged target-recognition element.

In some embodiments, such adaptor component may comprise at least one moiety that specifically recognizes and binds at least one tag of the tagged target-recognition element.

Thus, in certain embodiments, the target binding moiety of the CAR molecule of the present disclosure, may comprise antibodies or antigen binding fragments thereof, or any aptamer that are directed to two or more antigens (e.g., bi-specific, or tri-specific antibodies), or alternatively, different antibodies or any other affinity molecule/s that target/s the BCMA molecule.

In some embodiments, the adaptor component comprises at least one moiety that specifically recognizes and binds at least one tag of the tagged target-recognition element. According to such specific embodiments, the CAR molecule of the present disclosure may be adapted for various target recognition elements that are tagged by a tag recognized and by the adaptor component, thereby forming a recognition pair. These CAR molecules may be also indicated herein as universal CARs. In some specific embodiments, the recognition pair may include the biotin/avidin affinity pair. For example, the target recognition component may be tagged by biotin and attached to adaptor component of the disclosed CAR that comprises Streptavidin. It should be however appreciated that any other binding pairs are applicable for this purpose, for example, leucine zipper adaptor (zipCAR, and zipFv), Peptide neo-epitope (PNE) and anti-PNE, fluorescein (FITC) and anti-FITC, 10 amino acids (5B9 tag) and anti 5B9, FLAG, HA, SpyTag/SpyCatcher, Leucine zipper, SNAP-tag, CLIP-tag, Halo-tag, SpyTag, SnoopTag, Isopep-tag, and the like.

Still further, in some embodiments, the target-recognition domain and/or element of the CAR-molecule of the present disclosure comprises any affinity molecule that recognizes and binds the BCMA target molecule, and/or any other additional targets. The affinity molecule may comprise any ligand for the target molecule, any aptamer that specifically recognizes the BCMA or any other target molecule, any peptides that specifically interact with a particular antigen (e.g., peptibodies), receptor molecules that specifically interact with a particular antigen, proteins comprising a ligand-binding portion of a receptor that specifically binds a particular antigen or antigen-binding scaffolds, or at least one antibody or any antigen-binding fragment/s thereof. “Aptamers”, as used herein, refers in some embodiments, to peptide aptamer, that are small peptides with a single variable loop region tied to a protein scaffold on both ends that binds to a specific molecular target (e.g. protein), and which are bind to their targets only with said variable loop region and usually with high specificity properties.

In some specific embodiments, the target-recognition domain of the disclosed CAR molecule comprises at least one antibody or any antigen-binding fragment/s, portion/s or chimera/s thereof, specific for the BCMA.

The CAR molecule provided herein, comprises at least one target-binding domain, that may be in some embodiments, any target-recognition element, for example, at least one antibody or any antigen-binding fragments or domains thereof, as discussed herein above. In yet some further embodiments, the target-recognition element of the CAR molecule of the present disclosure comprises at least one antibody or any antigen-binding fragment/s, portion/s or chimera/s thereof. Exemplary categories of antigen-binding domains that can be used in the context of the present invention include antibodies, antigen-binding portions of antibodies (e.g., single chain variable fragments (scFv)), peptides that specifically interact with a particular antigen (e.g., peptibodies), receptor molecules that specifically interact with a particular antigen, proteins comprising a ligand-binding portion of a receptor that specifically binds a particular antigen or antigen-binding scaffolds. The antigen binding domains in accordance with the invention may recognize and bind a specific antigen or epitope. It should be therefore noted that the term “binding specificity”, “specifically binds to an antigen”, “specifically immuno-reactive with”, “specifically directed against” or “specifically recognizes”, when referring to an antigen or particular epitope, refers to a binding reaction which is determinative of the presence of the epitope in a heterogeneous population of proteins and other biologics. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three-dimensional structural characteristics as well as specific charge characteristics. Still further, as indicated above, an “antigen-binding domain” can comprise or consist of an antibody or antigen-binding fragment of an antibody such as single chain variable fragments (scFv). The term “antibody” as used herein, means any antigen-binding molecule or molecular complex comprising at least one complementarity determining region (CDR) that specifically binds to or interacts with a particular antigen or any epitope thereof. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

A typical antibody is composed of two immunoglobulin (Ig) heavy chains and two Ig light chains. In humans, antibodies are encoded by three independent gene loci, namely the immunoglobulin heavy locus (IgH) on chromosome 14, containing the gene segments for the immunoglobulin heavy chain, the immunoglobulin kappa (κ) locus (IgK) on chromosome 2, containing the gene segments for part of the immunoglobulin light chain and the immunoglobulin lambda (λ) locus (IgL) on chromosome 22, containing the gene segments for the immunoglobulin light chain.

The antibody and BCR heavy chains comprise 51 Variable (V) gene segments, 27 Diversity (D) gene segments, 6 Joining (J) gene segments. The antibody and BCR light chains comprise 40 Vκ, 31 Vλ, 5 Jκ, 4 Jλ gene segments.

Still further, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

Single domain antibodies also known as nanobodies (also known as Camelid single-domain antibodies or VHHs) have previously obtained by immunizing dromedaries, camels, llamas, alpacas, sharks, murine, rabbits and humans).

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In yet some further embodiments of the CAR-molecule of the present disclosure, the antigen-binding fragment/s, portion/s or chimera/s of such antibody comprises at least one of a single chain variable fragment (scFv), and/or nanobody. As used herein, single chain variable fragments (scFv) comprise the variable regions of the heavy (VH) and light chains (VL) of immunoglobulins, connected with a short linker peptide. Single-chain variable fragments lack the constant Fc region found in complete antibody molecules. Nevertheless, scFv retains the specificity of the original immunoglobulin, despite removal of the constant regions and the introduction of the linker.

The antibody suitable for the invention may also be a bi-specific antibody (or a tri-specific antibody. As indicated above, the antibody suitable for the invention may also be a variable new antigen receptor antibody (V-NAR), as well as any humanized forms thereof.

In some embodiments, the antibody is the mouse anti-BCMA antibody c11d5.3, as also described by (U.S. Pat. No. 9,034,324 B2, by Susan L. Kalled, Concord, MA and Yen-Ming Hsu), or any fragments thereof. This antibody comprises the heavy and light chain sequences as denoted by SEQ ID NO: 3 and SEQ ID NO: 5.

In some specific embodiments of the CAR-molecule of the present disclosure, the antibody specifically recognizes and binds the BCMA protein, or any fragments thereof. More specifically, such antibody comprises an immuno globulin heavy chain (HC) comprising the amino acid sequence as denoted by SEQ ID NO: 3, and any derivatives or variants thereof, and an immuno globulin light chain (LC) comprising the amino acid sequence as denoted by SEQ ID NO: 5, and any derivatives or variants thereof.

In yet some further embodiments, at least one target-binding domain of the CAR-molecule of the present disclosure comprises the amino acid sequence as denoted by SEQ ID NO: 11, and any derivatives or variants thereof.

The second component of the disclosed CAR molecule comprise the hinge region, and/or the transmembrane domain.

A Hinge region, or hinge domain as used herein is meant an extracellular flexible structure connecting between the targeting moiety and the T cell plasma membrane. These sequences are generally derived from IgG subclasses (such as IgG1 and IgG4), IgD and CD8 domains.

In yet some further embodiments, a “transmembrane region”, or transmembrane domain (TMD, also referred to herein as TM), of the disclosed CAR molecule, is a functional region of a protein that spans the phospholipid bilayer of a biological membrane, such as the plasma membrane of a cell. Integral membrane proteins typically comprise two or more such domains, alternating with intracellular and extracellular domains arranged on either side of the membrane. TMDs may consist predominantly of nonpolar amino acid residues and generally adopt an alpha helix conformation. Amino acids of the transmembrane domains interact with the fatty acyl groups of the membrane phospholipids, thereby anchoring the protein in the membrane.

In some embodiments, the hinge region may comprise between about 10 to 100, 20 to 90, 30 to 80, 30 to 70, 30 to 60 amino acid residues, specifically, about 25 to 50 aa, specifically, 25, 26, 27, 28, 29, 30, 31, 32, 33, 34, 35, 36, 37, 38, 39, 40, 41, 42, 43, 44, 45, 46, 47, 48, 49, 50, amino acid residues.

In some embodiments, the hinge region of the disclosed CAR molecule may comprise any sequence derived from the Cluster of Differentiation 8 α (CD8α) protein. The Cluster of differentiation 8 (CD8), also known as Ly-2 or Leu-2 and T-cell surface glycoprotein CD8 alpha chain, is a two chain and transmembrane glycoprotein which is expressed on the surface of circulating T-cells. CD8 serves as a co-receptor for the T cell receptor (TCR). Like the TCR, CD8 binds to a major histocompatibility complex (MHC) molecule but is specific for the class I MHC protein presented by antigen presenting cells (APCs). There are two isoforms of the CD8 protein, alpha and beta, each encoded by a different gene. CD8 exists as a disulfide-linked dimer either as α/β heterodimer or a α/α homodimer. In some embodiments, CD8α as used herein refers to the human CD8α homodimer. In some embodiments the human CD8α is denoted by Uniprot accession #P01732-1. Still further in some embodiments, the human CD8α comprises the amino acid sequence as denoted by SEQ ID NO: 26. In some embodiments, the hinge region of the disclosed CAR molecule may comprise the amino acid sequence as denoted by SEQ ID NO: 9, or any fragments, derivatives and variants thereof. Still further, in some embodiments, the disclosed hinge region of SEQ ID NO: 9 further comprises at least one residue, and is therefore, an example for a fragment of a hinge region. It some embodiments, the hinge region of SEQ ID NO: 9 further comprises a lysine residue. It yet some further embodiment, such hinge region is as denoted by SEQ ID NO: 35.

Still further, in some alternative embodiments, the hinge region of the disclosed CAR molecule may comprise the IgG4 hinge region. In some embodiments, a suitable hinge region may comprise the amino acid sequence as denoted by SEQ ID NO: 38. Still further, in some embodiments, the second component of the disclosed CAR molecule may comprise a transmembrane domain.

In some embodiments, the TM region may comprise between about 30 to 15 amino acid residues, specifically, about 25 to 17 aa, specifically, 25, 24, 23, 22, 21, 20, 19, 18, 17 amino acid residues. In some embodiments, the transmembrane domain may be derived from the CD8α protein.

In yet some further specific embodiments, the TM region used for the disclosed CAR molecule may comprise the amino acid sequence as denoted for SEQ ID NO: 10, or any fragments, derivatives and variants thereof.

In some alternative embodiments, the TM region of the disclosed car molecule may comprise a TM region derived from the CD28 molecule. Non-limiting embodiments for such TM, is the TM comprising the amino acid sequence as denoted by SEQ ID NO: 39.

In yet some further embodiments, the hinge and transmembrane domain of the CAR-molecule of the present disclosure, comprises the amino acid sequence as denoted by SEQ ID NO: 6, and any derivatives or variants thereof.

As shown by the disclosed Examples, the CAR T molecule of the present disclosure, specifically, the H8BB CAR molecule, display a clear superiority over the prior art CARs, for example, the H828 CAR T that shares similar target-binding domain, and/or similar TM and/or hinge regions that are derived from the same molecules, specifically, the CD8αtm, and Hinge. However, it should be noted that the hinge region of the disclosed CAR T, specifically, as denoted by SEQ ID NO: 9, differs from the hinge region of the H828 CAR T, that comprises the hinge region as denoted by SEQ ID NO: 22. Still further, in some embodiments, a hinge region useful for the CAR molecules of the present disclosure comprises the amino acid sequence as denoted by SEQ ID NO: 35.

Still further, the third component of the CAR molecule of the present disclosure is at least one signal transduction domain. As used herein, the term signal transduction domain, refers in some embodiments to the functional, intracellular portion of a receptor protein that acts to transmit the detected stimulatory information within the cell, thereby regulating the cellular activity through specific signaling pathways. According to some embodiments, this domain is an intracellular domain connected to the transmembrane domain, specifically, the TM domain used by the CAR molecules of the present disclosure.

Still further, in some embodiments, the at least one intracellular T cell signal transduction domain of the CAR-molecule of the present disclosure, comprises at least one tumor necrosis factor (TNF) receptor family member. The tumor necrosis factor receptor (TNFR) superfamily members, are membrane bound or soluble receptors which interact with membrane-bound and/or soluble ligands of the TNF superfamily. The majority of the members of this TNF/TNFR superfamily is expressed by immune cells. Activation of the TNFR members via their ligands affects cell proliferation, survival, differentiation and apoptosis of responding cells.

The TNF-like receptors are type I transmembrane proteins characterized by cysteine-rich domains (CRD) that are the hallmark of the TNFR superfamily. These pseudorepeats are defined by intrachain disulphides generated by highly conserved cysteine residues within the receptor chains. The members of the tumour necrosis factor (TNF)/tumour necrosis factor receptor (TNFR) superfamily are critically involved in the maintenance of homeostasis of the immune system. The biological functions of this system encompass beneficial and protective effects in inflammation and host defense as well as a crucial role in organogenesis. At the same time, members of this superfamily are responsible for host damaging effects in sepsis, cachexia, and autoimmune diseases. The TNFR superfamily includes for example TNFR1 (also sometimes referred to a p55/p60), TNFR2 (also known as p75/p80) and B-cell activating factor receptor (BAFFR). Still further, the tumor necrosis factor (TNF) family includes for example TNF alpha (TNFα), TNF beta (TNFβ), CD40 ligand (CD40L), Fas ligand (FasL), TNF-related apoptosis inducing ligand (TRAIL), and LIGHT (is homologous to lymphotoxins, exhibits inducible expression, and competes with HSV glycoprotein D for HVEM, a receptor expressed by T lymphocytes), some of the most important cytokines involved in physiological processes, systematic inflammation, tumor lysis, apoptosis and initiation of the acute phase reaction.

In some embodiments, a TNF receptor family member useful as a signal transduction intracellular domain of the CAR molecule of the present disclosure is the 4-1BB. Thus, in some embodiments, the CAR molecule of the preset disclosure compromises an intracellular domain derived from the 4-1BB. The 4-1BB (TNFRSF9, CD137) is an activation-induced T cell costimulatory molecule, and a TNFR superfamily member. 4-1BB is expressed on a subset of resting CD8⁺ T cells and is upregulated on both CD4⁺ and CD8⁺ T cells following activation. Upon binding to trimeric 4-1BBL (TNFSF9, CD137L) on APCs, 4-1BB recruits TNFR-associated factor family members (TRAF1, TRAF2 and TRAF3) to its cytosolic region, forming the 4-1BB signalosome and leading to downstream activation of NF-κB, MAPK and ERK. Agonistic stimulation of 4-1BB upregulates expression of the anti-apoptotic proteins Bcl-x_{L} and Bfl-1. Still further, 4-1BB activation increases IL-2 and IFN-γ in CD8⁺ cells and IL-2 and IL-4 in CD4⁺ cells. T cells expressing CARs that incorporate 4-1BB domains have been shown to express granzyme B, IFN-γ, TNF-α, GM-CSF and the anti-apoptotic protein Bcl-x_{L}. Still further, incorporation of the 4-1BB TM and cytoplasmic domain into a CAR, leads to improved persistence and antitumor activity, as well as to prolonged T cell division.

In some embodiments, 4-1BB as used herein refers to the human 4-1BB. In some embodiments, the human 4-1BB is as denoted by Uniprot accession #Q07011. In some further embodiments, the human 4-1BB comprises the amino acid sequence as denoted by SEQ ID NO: 28, and any variants and derivatives thereof.

In yet some further optional embodiments, the at least one intracellular T cell signal transduction domain of the of the CAR-molecule of the present disclosure, further comprises at least one TCR molecule or any fragments thereof. More specifically, the T-cell receptor (TCR) is a protein complex found on the surface of T cells, or T lymphocytes, that is responsible for recognizing fragments of antigen as peptides bound to major histocompatibility complex (MHC) molecules. When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors.

The core TCR complex consists of two TCR chains and six cluster of differentiation 3 (CD3) chains. The human genome expresses four TCR genes known as TCRα, TCRβ, TCRγ, and TCRδ, which forms two distinct heterodimers: TCRα/TCRβ or TCRγ/TCRδ. The majority of mature T cells expresses TCRα and TCRβ isoforms, generally referred to as T cells (or αβ T cells), while a small portion (0.5-5%) of T lymphocytes (γδ T cells) expresses TCRγ and TCRδ isoforms. Both heterodimers form multiprotein complexes with CD3 δ, γ, ε, and ζ chains. However, in both complexes, three dimers of CD3 proteins, δε and γε heterodimers and ζζ homodimers, are present. These CD3 proteins associate with TCR via non-covalent hydrophobic interactions and are required for a complete TCR localization on the cell surface. The TCR mediates recognition of antigenic peptides bound to MHC molecules (pMHC), whereas the CD3 molecules transduce activation signals to the T cell.

In more specific embodiments, the CAR molecule of the present disclosure may comprise at least one region derived from at least one domain of the TCR, specifically, the cluster of differentiation 3 (CD3) zeta chain. Thus, in some embodiments, the CAR molecule of the preset disclosure compromises an intracellular domain that further comprises a domain derived from the CD3-ζ. T-cell surface glycoprotein CD3 zeta chain also known as T-cell receptor T3 zeta chain or CD247 (Cluster of Differentiation 247) is a protein encoded in human by the CD247 gene. More specifically, CD3 (cluster of differentiation 3) T-cell co-receptor helps to activate the cytotoxic T-cell. It consists of a protein complex and is composed of four distinct chains. In mammals, the complex contains a CD3γ chain, a CD3δ chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex. T-cell receptor zeta, together with T-cell receptor alpha/beta and gamma/delta heterodimers and CD3-gamma, -delta, and -epsilon, forms the T-cell receptor-CD3 complex. The zeta-chain plays an important role in coupling antigen recognition to several intracellular signal-transduction pathways and is thus included in the CAR T molecule of the present disclosure. In some embodiments, CD3 zeta as used herein refers to the human CD3 zeta. In some embodiments, the human CD3 zeta is as denoted by Uniprot accession #P20963-1. Still further, in some embodiments, the human CD3 zeta used in the present disclosure may comprise the amino acid sequence as denoted by SEQ ID NO: 27, and any derivatives or variants thereof.

In some specific and non-limiting embodiments, the at least one intracellular T cell signal transduction domain of the CAR-molecule of the present disclosure comprises the amino acid sequence as denoted by SEQ ID NO: 12, or any variants and derivatives thereof.

In yet some further specific embodiments, of the CAR-molecule of the present disclosure comprises the amino acid sequence as denoted by SEQ ID NO: 1, or any variants and derivatives thereof. It should be noted that in some embodiments, the CAR T of the present disclosure is also referred to herein as H8BB CART, as well as HBI0101 (clinical grade as used herein) CART.

It should be further understood that the present disclosure further encompasses any of the disclosed CAR T molecules, specifically, the ICBB CAR molecule that comprises the amino acid sequence as denoted by SEQ ID NO: 36. Still further, the present disclosure further encompasses the IC28 CAR molecule that comprises the amino acid sequence as denoted by SEQ ID NO: 37.

In yet some further embodiments, the expression of the CAR-molecule of the present disclosure by at least one cell of the T lineage results according to some alternative or additional embodiments, in several features that distinguish the disclosed CAR molecule from any prior art CAR molecule, and moreover, define the superiority of the disclosed CAR molecule over the prior art CAR molecules. More specifically, in some embodiments (i), the disclosed CAR molecule displays, or is characterized by increased specificity to the target. Particularly, increased specificity to any cells that express the target, BCMA.

In some embodiments, specificity as used herein refers to the ability of the CAR molecule to activate the expressing T cells in response to a specific antigen. While inducing no, or a negligible response toward cells expressing a different antigen.

In yet some further additional or alternative embodiments, the expression of the CAR-molecule of the present disclosure by at least one cell of the T lineage results in (ii), reduced tonic signaling. Thus, in some embodiments, the disclosed CAR molecule displays, or is characterized by reduced tonic signaling. More specifically, as used herein, the term Tonic signaling can be defined as a constitutive or chronic activation of T cells in the absence of a ligand. Physiologically, low-level continuous tonic signaling via interactions between the endogenous TCR and self-peptide-loaded MHC molecules constitutes an important mechanism to regulate T cell homeostasis. In contrast, tonic signaling mediated by T cell-engrafted CAR constructs appears to be more complex. Tonic signaling may be influenced by the CAR configuration as demonstrated in FIG. 2 and FIG. 3C. Still further, in some additional or alternative embodiments, the expression of the CAR-molecule of the present disclosure by at least one cell of the T lineage results in (iii), reduced off-target activation. Thus, in some embodiments, the disclosed CAR molecule displays, or is characterized by reduced off-target activation. More specifically, Off-target activation refers herein to non-specific cytokine secretion in control co-cultures with an antigen-negative target (such as K562) or without any target as demonstrated in FIG. 2 and FIG. 4. Off-target activation may be influenced by the CAR configuration.

In some further additional or alternative embodiments, the expression of the CAR-molecule of the present disclosure by at least one cell of the T lineage results in (iv), increased expression of activation markers in response to a specific stimulation. Thus, in some embodiments, the disclosed CAR molecule displays, or is characterized by increased expression of activation markers. In yet some further embodiments, the disclosed CAR molecules increase the activation of T cells. T cells are generated in the Thymus and are programmed to be specific for one particular foreign particle (antigen). Once they leave the thymus, they circulate throughout the body until they recognize their antigen on the surface of antigen presenting cells (APCs). The T cell receptor (TCR) on both CD4+ helper T cells and CD8+ cytotoxic T cells binds to the antigen as it is held in a structure called the MHC complex, on the surface of the APC. This triggers initial activation of the T cells. The CD4 and CD8 molecules then bind to the MHC molecule too, stabilizing the whole structure. This initial binding between a T cell specific for one antigen and the antigen-MHC it matches sets the whole response in motion. This normally takes place in the secondary lymphoid organs.

In addition to TCR binding to antigen-loaded MHC, both helper cells and cytotoxic T cells require a number of secondary signals to become activated and respond to the threat. In the case of helper T cells, the first of these is provided by CD28. This molecule on the T cell binds to one of two molecules on the APC-B7.1 (CD80) or B7.2 (CD86) - and initiates T-cell proliferation. This process leads to the production of many millions of T cells that recognize the antigen. In order to control the response, stimulation of CD28 by B7 induces the production of CTLA-4 (CD152). This molecule competes with CD28 for B7 and so reduces activation signals to the T cell and winds down the immune response. Cytotoxic T cells are less reliant on CD28 for activation but do require signals from other co-stimulatory molecules such as CD70 and 4-1BB (CD137). T cells must recognize foreign antigen strongly and specifically to mount an effective immune response and those that do are given survival signals by several molecules, including ICOS, 4-1BB and OX40. These molecules are found on the T-cell surface and are stimulated by their respective ligands which are typically found on APCs. Unlike CD28 and the TCR, ICOS, OX40 and 4-1BB are not constitutively expressed on T cells. Likewise, their respective ligands are only expressed on APCs following pathogen recognition. This is important because it ensures T cells are only activated by APCs which have encountered a pathogen and responded. Interaction of the TCR with peptide-MHC in the absence of co-stimulation switches the T cells off, so they do not respond inappropriately. Once the T cell has received a specific antigen signal and a general signal two, it receives more instructions in the form of cytokines. These determine which type of responder the cell will become—in the case of helper T cells, it will push them into Th1 type (cells exposed to the cytokine IL-12), Th2 (IL-4), or IL-17 (IL-6, IL-23). Each one of these cells performs a specific task in the tissue and in developing further immune responses. As indicated above, increased activation of T cells may be reflected for example, by the increase in the expression of activation markers. Activation markers refer herein to molecules which are upregulated upon T cell activation, each at a different stage of the activation process. For example, the earliest activation marker is CD69, which is an inducible cell surface glycoprotein expressed upon activation via the TCR or the IL-2 receptor (CD25). It plays a role in the proliferation and survival of activated T lymphocytes.

According to some embodiments, increase in the expression of activation markers, may comprise for example, increase in the expression of at least one of Leukocyte antigen 37 (CD37), CD25 (also known as the αchain of the high affinity IL-2 receptor), Cluster of differentiation 69 (CD69). It should be further understood that specific stimulation as used herein, is the presence of BCMA-positive target cells. Still further in some additional or alternative embodiments, the expression of the CAR-molecule of the present disclosure by at least one cell of the T lineage results in (v), reduced expression of exhaustion markers in response to a specific stimulation. T cell Exhaustion as used herein refers to a state of T cell dysfunction that arises during many chronic infections and cancer. It is defined by poor effector function, sustained expression of inhibitory receptors and a transcriptional state distinct from that of functional effector or memory T cells. By “dysfunction” here it is understood that some T cells, after activation and proliferation, do not fulfill the functions they are expected to perform as effector T cells-typically, they fail to eliminate cancerous or infected cells and control the tumor or the virus respectfully. As originally described, antigen-specific T cells become “dysfunctional” during the chronic phase of high viral load infections, with progressive loss of interleukin (IL)-2, then tumor necrosis factor alpha (TNFα), and, finally, interferon gamma (IFNγ). Thus, in some embodiments, the disclosed CAR molecule displays, or is characterized by reduced expression of exhaustion markers. More specifically, in some embodiments, the exhaustion markers may be at least one of Programmed Death-1 receptor (PD-1), Lymphocyte activation gene-3 (LAG-3, is also named CD223 or FDC protein), T-cell immunoglobulin and mucin-domain containing-3 (TIM3), T cell immunoreceptor with Ig and ITIM domains (TIGIT), as also disclosed by FIG. 3C, in an in vivo and/or in vitro ex vivo setting. Still further, in some additional and non-limiting embodiments, exhaustion markers applicable in the present disclosure include inducible T-cell co-stimulator (ICOS), cytotoxic T-lymphocyte-associated protein-4 (CTLA-4), CD244 (2B4), CD160, killer cell lectin-like receptor subfamily G member 1 (KLRG1), and the like.

As indicated above, one of the major and advantageous properties displayed by the disclosed CAR molecule is the reduced tonic signaling. More specifically, tonic signaling can be dependent or independent of the relevant Ag and has major effects on CAR based therapies. On the one hand, CAR tonic signaling may promote T cell expansion by providing stimulating signals. On the other hand, CAR tonic signaling can trigger terminal effector T cell differentiation, exhaustion and/or enhanced activation-induced cell death, and, therefore, limits in vivo persistence as well as antitumor potential. Therefore, reduction thereof, as shown by the CAR T molecules of the present disclosure, increases specificity and effectivity of the response.

As indicated above, the CAR molecules of the present disclosure display superior properties that are characterized by at least one of reduced tonic signaling; reduced off-target activation; reduced expression of exhaustion markers in response to a specific stimulation, and increased specificity and increased expression of activation markers in response to a specific stimulation, in an in vivo and/or in vitro ex vivo setting. Increase, as used herein, in connection with various improved properties of the CAR molecule of the present disclosure, is meant that such increase or enhancement may be an increase or elevation of the indicated activity (e.g., specificity, expression of activation markers and the like), of between about 1% to 100%, specifically, 5% to 100% of the indicated parameter, more specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. In yet some further embodiments, the terms “inhibition”, “moderation”, “reduction”, “decrease” or “attenuation” as referred to herein with respect to the various properties of the CAR molecule of the preset disclosure (e.g., expression of exhaustion markers, reduced tonic signaling), relate to the retardation, restraining or reduction of the indicated parameter by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. More specifically, the terms “increase”, “augmentation” and “enhancement” as used herein relate to the act of becoming progressively greater in size, amount, number, or intensity. Alternatively, “inhibition”, “moderation”, “reduction”, “decrease” or “attenuation” as used herein relate to the act of becoming progressively smaller in size, amount, number, or intensity. Particularly, an increase or alternatively, decrease of 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of the indicated activity (e.g., increase of specificity and/or expression of activation markers, or alternatively, decrease of expression of exhaustion markers and/or tonic signaling) as compared to a suitable control, e.g., in the absence of the CAR molecule of the present disclosure, or in comparison with other CAR molecules, for example, any of the other (e.g. reference, H828) CAR molecules disclosed herein.

More specifically, the CAR molecule of the present disclosure displays a significantly decreased exhaustion. In some embodiments, the decreased exhaustion is reflected by decrees in the expression of various exhaustion markers. More specifically, as demonstrated by the Examples, the exhaustion profile of HBI0101 (H8BB) CART cells in resting cells was dramatically lower than the exhaustion profile of H828 CART cells; i.e. the expression of PD1, LAG3, TIM3 and TIGIT T-cell exhaustion markers at the surface of HBI0101 (H8BB) CART cells was reduced by 79% (ranging between 72%-86%), 68% (ranging between 59%-74%), 77% (ranging between 69%-84%), 43% (ranging between 37%-47%) in comparison with the prior art H828 CART cells, respectively.

Exhaustion profile of HBI0101 (H8BB) CART cells activated with BCMA-expression NCI-H929 cells was significantly lower than the exhaustion profile of H828 CART cells activated with the same BCMA-expression NCI-H929 cells; i.e. the expression of PD1, LAG3, TIM3 and TIGIT T-cell exhaustion markers at the surface of target-activated HBI0101 (H8BB) CART cells was reduced by 64% (ranging between 59%-69%), 54% (ranging between 43%-66%), 58% (ranging between 50%-63%) and 55% (ranging between 50%-58%) in comparison with target-activated the prior art H828 CART cells, respectively.

The exhaustion profile of HBI0101 (H8BB) CART cells following activation with BCMA-expression K562-BCMA overexpressing cells was significantly lower than the exhaustion profile of H828 CART cells activated with the same BCMA-expression NCI-H929 cells; i.e. expression of PD1, LAG3, TIM3 and TIGIT T-cell exhaustion markers at the surface of target-activated HBI0101 (H8BB) CART cells were reduced by 55% (ranging between 47%-63%), 41% (ranging between 31%-43%), 55% (ranging between 50%-61%), 38% (ranging between 33%-42%), in comparison with target-activated H828 CART cells, respectively. Thus, in some embodiments, the CAR T cells of the present disclosure display a reduction of about 30% to about 90%, about 35% to 80%, about 38% to 80%, in the expression of exhaustion markers i.e. PD1, LAG3, TIM3 and TIGIT. Specifically, a decrease of about 30%, 35%, 40%, 45%, 50%, 55%, 60%, 65%, 70%, 75%, 80%, 85%, 90% or more, e.g., 95%, 100%. More specifically, in some embodiments, a decrease in about 79% 64%, 55% of PD1 expression. In certain embodiments, a decrease of about 68%, 54%, 41%, in the expression of LAG3. In yet some further embodiments, the decrease is of about 77%, 58%, 55%, in the expression of TIM3. In yet some further embodiments, the decrease is of about 43%, 55% or 38%, in the expression of TIGIT. It should be understood that in some embodiments, the decrease indicated herein, is as compared with the prior art CAR T cells (e.g., H828 CART cells). Still further, as shown by the examples, the CAR T cells of the present disclosure display increased cytotoxicity against BCMA-expressing target cells. In some embodiments, the cytotoxicity is increased in about 50% to about 100% or more, specifically, about 50%, 55%, 60%, 65%, 70%, 75%, 80%, 85%, 90%, 95%, 100% cytotoxicity as compared with the prior art (H828) CAR T cells. In some embodiments, an increase of about 57%, or of 75% or more.

In yet some further embodiments, an increase in activation maker may range between about 50% to about 100%, as compared with the prior art (H828) CAR T cells. Specifically, 50%, 55%, 60%, 65%, 70%, 75%, 80%, 85%, 90%, 95%, 100%, for example, an increase of about 69% or more in the expression of activation markers, for example, CD69.

In some embodiments, the expression of the CAR molecule of the present disclosure by at least one cell of the T lineage of a subject suffering from an immune-related disorder, results in modulation of the immune-response in the subject.

Still further, in some embodiments, such immune related disorder may be any immune-related disorder associated with increased expression of the BCMA marker. In yet some further embodiments, such immune-related disorder may be at least one plasma cell pathology. In some embodiments, such pathologies may include at least one proliferative disorder, and/or at least one deposit disorder, and/or at least one autoimmune disease.

In yet some further alternative embodiments, the CAR molecule of the present disclosure may be applicable for an inflammatory disorder, an autoimmune disorder, an infectious disease caused by a pathogen, a neurodegenerative disease, a congenital disorder, an allergic condition, a cardiovascular disease, immuno deficiency (acquired or inherited) and a metabolic condition. Specific examples for disorders applicable for the present disclosure or any aspects thereof, is disclosed herein after in connection with other aspects of the invention.

The present disclosure provides CAR T molecules that are composed of amino acid residues and are therefore a polypeptide. The term “polypeptide” as used herein refers to amino acid residues, connected by peptide bonds. A polypeptide sequence is generally reported from the N-terminal end containing free amino group to the C-terminal end containing free carboxyl group and may include any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that contains portions that occur in nature separately from one another (i.e., from two or more different organisms, for example, human and non-human portions). In some embodiments, a polypeptide has an amino acid sequence that is engineered in that it is designed and/or produced through action of the hand of man. More specifically, “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, or protein sequence, if it represents the primary structure of a protein. However, one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

It should be appreciated that the invention encompasses the use of any variant or derivative of the polypeptides of the invention, specifically any polypeptide comprising at least one of the amino acid sequences as denoted by any one of SEQ ID NO: 1, or any fragments thereof, for example, the CAR T molecule of SEQ IS NO; 40 that does not contain the leader sequence, or any parts thereof, as denoted by SEQ ID NOs: 2 to 12, or any derivatives thereof, and any polypeptides that are substantially identical or homologue to the polypeptides encoded by the nucleic acid sequence of the invention, as indicated herein above. The term “derivative” is used to define amino acid sequences (polypeptide), with any insertions, deletions, substitutions and modifications to the amino acid sequences (polypeptide) that do not alter the activity of the original polypeptides, e.g., at least one of: increased specificity, reduced tonic signaling, reduced off-target activation, increased expression of activation markers in response to a specific stimulation, and/or reduced expression of exhaustion markers in response to a specific stimulation, in an in vivo and/or in vitro ex vivo setting, as discussed above. By the term “derivative” it is also referred to homologues, variants and analogues thereof. Proteins orthologs or homologues having a sequence homology or identity to the proteins of interest in accordance with the invention, specifically, receptors, chimeras and antibodies described herein, may share at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the proteins of interest in accordance with the invention, specifically, any one of SEQ ID NO: 1, or any fragments thereof, for example, the CAR T molecule of SEQ IS NO: 40 that does not contain the leader sequence, or any parts thereof, as denoted by SEQ ID NOs: 2 to 12, or any derivatives thereof.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention as indicated above, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof. With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention. For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q). Variants of the polypeptides of the present disclosure may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the various polypeptides of the invention. It should be understood that the percentage of similarity or identity refer to the similarity or identity to the entire sequences as denoted by any one of SEQ ID NO: 1, or any fragments thereof, for example, the CAR T molecule of SEQ IS NO: 40 that does not contain the leader sequence, or any parts thereof, as denoted by SEQ ID NOs: 2 to 12, and any variants or derivatives thereof.

A further aspect of the present disclosure relates to a nucleic acid molecule comprising at least one nucleic acid sequence encoding at least one CAR molecule, or any cassette, vector or vehicle comprising the nucleic acid molecule. In some embodiments, such encoded CAR molecule comprises the following components. First (i), at least one target-binding domain; wherein at least one of said target binding domain specifically recognizes and binds BCMA; second (ii), at least one hinge and at least one transmembrane domain derived from the CD8α protein. It should be noted that the hinge region of the domain comprises the amino acid sequence as denoted by SEQ ID NO:9, and any fragments, derivatives and variants thereof. The CAR molecule further comprises as a third component (iii), at least one intracellular T cell signal transduction domain. More specifically, this domain comprises at least one domain of TNF receptor family member, and optionally, at least one domain of a TCR molecule.

In some embodiments, the present disclosure provides any nucleic acid molecule encoding any of the CAR molecule as defined by the present disclosure.

Still further, in some embodiments, the nucleic acid molecule of the present disclosure may be flanked on at least one of the 5′ and 3′ ends thereof by at least one of: (i) homology arms, for integration to a genomic target site by homologous recombination; and/or (ii) recognition sites for a site-specific nuclease, a site-specific integrase or a site-specific recombinase. The term “flanked” as used herein refers to a nucleic acid sequence positioned between two defined regions. For example, as indicated above, the nucleic acid molecule of the present disclosure that encodes the CAR is flanked by at least one homology arm/s and/or recognition sites, positioned 5′ (or upstream) and/or 3′ (or downstream) to the nucleic acid molecule of the present disclosure. Homology arms, as used herein, are genomic DNA fragment/s, located at the 5′ and/or the 3′ of a nucleic acid sequence, also referred to herein as 5′- and 3′-homology arms (or simply 5′ and 3′ arms, or left and right arms) flanking at least one nucleic acid sequence of interest (e.g., in a donor cassette). The arms homologously recombined with the complementary sequence/s that flank a target nucleic acid sequence to achieve successful genetic modification of the target nucleic acid sequence.

The invention involves the provision of a nucleic acid molecule encoding the disclosed CAR, that in some embodiments may be, and/or comprised within, a cassette, that is used in the methods, cells, compositions and uses described in all aspects of the invention. The term “nucleic acid cassette” refers to a polynucleotide sequence comprising at least one regulatory sequence operably linked to a sequence encoding a nucleic acid sequence encoding the CAR Ts disclosed herein. All elements comprised within the cassette of the invention are operably linked together. The term “operably linked”, as used in reference to a regulatory sequence and a structural nucleotide sequence, means that the nucleic acid sequences are linked in a manner that enables regulated expression of the linked structural nucleotide sequence. It should be appreciated that any nucleic acid cassette provided herein comprises the nucleic acid sequence disclosed herein (e.g., the sequence encoding the CAR T of the invention as denoted by SEQ ID NO: 13, and any homologs and variants thereof, or the encoding sequence for any fragments thereof as denoted by SEQ ID NOs: 14 to 20, and any homologs and variants thereof). According to some embodiments, such nucleic acid cassette may further comprise any control sequences that facilitate the transcription and/or translation of the CAR T molecules of the preset disclosure. Such sequences include, as non-limiting examples, a promoter sequence, specifically, a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present invention.

In some embodiments, promoters applicable in the present invention may be either inducible or constitutive. In yet some further embodiments, minimal promoter may be used, still further, endogenous promoter or heterologous promoter are also applicable in the cassettes disclosed herein. In some embodiments, the cassettes of the present disclosure may further comprise a Signal peptide leader, for example, a signal peptide leader derived from CD8, as used herein, specifically, of SEQ ID NO: 2, and any derivatives and variants thereof. In yet some further embodiments, the nucleic acid molecules encoding the CAR provided by the invention may further comprise at least one degron sequence, at least one 2A peptide sequence or a CHYSEL site, at least one mRNA stabilizing sequence, at least one stop codon (or termination codon), at least one 3-frame stop codon sequence, at least one protein stabilizing sequence, at least one polyadenylation sequence at least one transcription enhancer, splice donor and/or splice acceptor sites, and any transcription and/or translation element/s.

The invention provides nucleic acid molecules, sequences encoding the encoding the immune effector of interest and the engineered CAR T of the present disclosure, cassette, and methods, cells, uses and compositions thereof. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art. Still further, it should be understood that the invention encompasses as additional aspects thereof any vector or vehicle that comprise any of the nucleic acid molecule/s of the invention or any cassettes described by the invention.

Still further, in some embodiments, the nucleic acid molecule/s of the invention or any cassette used by the invention may be comprised within a nucleic acid vector. In more specific embodiments, such vector may be any one of a viral vector, a non-viral vector and a naked DNA vector.

Vectors, as used herein, are nucleic acid molecules of particular sequence can be incorporated into a vehicle that is then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g., plasmids, cosmids, minicircles, phage, viruses, etc., useful for transferring nucleic acids into target cells may be applicable in the present invention. The vectors comprising the nucleic acid(s) may be maintained episomally, e.g., as plasmids, minicircle DNAs, viruses such cytomegalovirus, adenovirus, etc., or they may be integrated into the target cell genome, through homologous recombination or random integration, e.g., retrovirus-derived vectors such as AAV, MMLV, HIV-1, ALV, etc. Vectors may be provided directly to the subject cells. In other words, the cells are contacted with vectors comprising the nucleic acid molecules, and/or cassettes of the invention that comprise the nucleic acid sequence encoding the encoding the immune effector of interest and the engineered CAR T disclosed herein such that the vectors are taken up by the cells. Methods for contacting cells with nucleic acid vectors that are plasmids, such as electroporation, calcium chloride transfection, and lipofection, are well known in the art. DNA can be introduced as naked nucleic acid, as nucleic acid complexed with an agent such as a liposome or poloxamer, or can be delivered by viruses (e.g., adenovirus, AAV). More specifically, in some embodiments, the vector may be a viral vector. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and l'lentiviral vector. As indicated above, in some embodiments, viral vectors may be applicable in the present invention. The term “viral vector” refers to a replication competent or replication-deficient viral particle which are capable of transferring nucleic acid molecules into a host. The term “virus” refers to any of the obligate intracellular parasites having no protein-synthesizing or energy-generating mechanism. The viral genome may be RNA or DNA contained with a coated structure of protein of a lipid membrane. Examples of viruses useful in the practice of the present invention include baculoviridiae, parvoviridiae, picornoviridiae, herepesviridiae, poxviridiae, adenoviridiae, picotmaviridiae. The term recombinant virus includes chimeric (or even multimeric) viruses, i.e., vectors constructed using complementary coding sequences from more than one viral subtype.

In some embodiments, the nucleic acid molecules, and/or cassette of the invention may be comprised within a retroviral vector. A retroviral vector, as used herein consists of proviral sequences that can accommodate the nucleic acid molecule encoding the engineered CAR T disclosed herein, to allow incorporation of both into the target cells. The vector may also contain viral and cellular gene promoters, to enhance expression of the nucleic acid molecule encoding the encoding the immune effector of interest and the engineered CAR T disclosed herein in the target cells. Retroviral vectors stably integrate into the dividing target cell genome so that the introduced gene is passed on and expressed in all daughter cells. They contain a reverse transcriptase that allows integration into the host genome.

In some specific and non-limiting embodiments, the pMSGV1 retroviral vector has been used for the CAR T molecule of the present disclosure. More specifically, the pMSGV1 retroviral vector contains a murine stem cell virus long-terminal repeat and RNA processing signals similar to the MFG class of retroviral vectors. The retroviral vector backbone used in this the present disclosure, pMSGV1, is a derivative of the vector pMSGV (MSCV-based splice-gag vector) that utilizes a murine stem cell virus (MSCV) long terminal repeat (LTR) [Hawley et al., Gene Ther. 1:136-138 (1994), and contains the extended gag region and env splice site from vector SFGtcLuc+ITE4 [Lindemann et al., Mol. Med. 1997; 3:466-476 (1997)]. Vector pMSGV was generated from pMINV [Hawley et al., Ann. N.Y. Acad. Sci. 795:341-345. (1996)] by substituting a 756-bp SpeI/XhoI fragment with a 798-bp SpeI/XhoI fragment from SFGtcLuc+ITE4 and by replacing a 1955-bp XhoI/BamHI fragment containing a PGK-IRES-NEO cassette with a 47-bp XhoI/BamHI polylinker containing unique XhoI, EcoRI, SalI, SacII, and BamHI sites. Vector pMSGV1 was derived from pMSGV by replacing a 43-bp PmlI/XhoI fragment of pMSGV with a 76-bp PmlI/XhoI fragment from the vector Gcsap [Onodera et al., J. Virol. 72:1769-1774 (1998)]. The latter modification incorporates a naturally occurring Kozak sequence to enhance translational efficiency. In some alternative embodiments, the nucleic acid molecules, and/or cassette of the invention may be comprised within an Adeno-associated virus (AAV). The term “adenovirus” is synonymous with the term “adenoviral vector”. AAV is a single-stranded DNA virus with a small (˜20 nm) protein capsule that belongs to the family of parvoviridae, and specifically refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (AdllA and Ad IIP), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. Due to its inability to replicate in the absence of helpervirus coinfections (typically Adenovirus or Herpesvirus infections) AAV is often referred to as dependovirus. AAV infections produce only mild immune responses and are considered to be nonpathogenic, a fact that is also reflected by lowered biosafety level requirements for the work with recombinant AAVs (rAAV) compared to other popular viral vector systems. Due to its low immunogenicity and the absence of cytotoxic responses AAV-based expression systems offer the possibility to express nucleic acid sequences encoding the encoding the immune effector of interest and the engineered CAR T disclosed herein for months in quiescent cells. Production systems for rAAV vectors typically consist of a DNA-based vector containing a transgene expression cassette, which is flanked by inverted terminal repeats. Construct sizes are limited to approximately 4.7-5.0 kb, which corresponds to the length of the wild-type AAV genome. rAAVs are produced in cell lines. The expression vector is co-transfected with a helper plasmid that mediates expression of the AAV rep genes which are important for virus replication and cap genes that encode the proteins forming the capsid. Recombinant adeno-associated viral vectors can transduce dividing and non-dividing cells, and different rAAV serotypes may transduce diverse cell types. These single-stranded DNA viral vectors have high transduction rates and have a unique property of stimulating endogenous Homologous Recombination without causing double strand DNA breaks in the host genome.

It should be appreciated that many intermediate steps of the wild-type infection cycle of AAV depend on specific interactions of the capsid proteins with the infected cell. These interactions are crucial determinants of efficient transduction and expression of nucleic acid molecules encoding the encoding the immune effector of interest and the engineered CAR T disclosed herein when rAAV is used as gene delivery tool. Indeed, significant differences in transduction efficacy of various serotypes for particular tissues and cell types have been described.

It is believed that a rate-limiting step for the AAV-mediated expression of transgenes is the formation of double-stranded DNA. Recent reports demonstrated the usage of rAAV constructs with a self-complementing structure (scAAV) in which the two halves of the single-stranded AAV genome can form an intra-molecular double-strand. This approach reduces the effective genome size usable for gene delivery to about 2.3 kB but leads to significantly shortened onsets of expression in comparison with conventional single-stranded AAV expression constructs (ssAAV). Thus, in some embodiments, ssAAV may be applicable as a viral vector by the methods of the invention.

In yet some further embodiments, HDAd vectors may be suitable for the CAR molecules, encoding sequences, cells, compositions and methods of the present disclosure. The Helper-Dependent Adenoviral (HDAd) vectors HDAds have innovative features including the complete absence of viral coding sequences and the ability to mediate high level transgene expression with negligible chronic toxicity. HDAds are constructed by removing all viral sequences from the adenoviral vector genome except the packaging sequence and inverted terminal repeats, thereby eliminating the issue of residual viral gene expression associated with early generation adenoviral vectors. HDAds can mediate high efficiency transduction, do not integrate in the host genome, and have a large cloning capacity of up to 37 kb, which allows for the delivery of multiple transgenes or entire genomic loci, or large cis-acting elements to enhance or regulate tissue-specific transgene expression. One of the most attractive features of HDAd vectors is the long-term expression of the transgene. Still further, in some embodiments, SV40 may be used as a suitable vector by the methods of the invention. SV40 vectors (SV40) are vectors originating from modifications brought to Simian virus-40 an icosahedral papovavirus. Recombinant SV40 vectors are good candidates for gene transfer, as they display some unique features: SV40 is a well-known virus, non-replicative vectors are easy-to-make, and can be produced in titers of 10(12) IU/ml. They also efficiently transduce both resting and dividing cells, deliver persistent transgene expression to a wide range of cell types, and are non-immunogenic. Present disadvantages of rSV40 vectors for gene therapy are a small cloning capacity and the possible risks related to random integration of the viral genome into the host genome. In yet some alternative embodiments, lentiviral vectors may be used in the present invention. Lentiviral vectors are derived from lentiviruses which are a subclass of Retroviruses. Commonly used retroviral vectors are “defective”, i.e., unable to produce viral proteins required for productive infection. Rather, replication of the vector requires growth in a packaging cell line. To generate viral particles comprising the nucleic acid molecules, vectors and/or cassette in accordance with the invention, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acid molecules, vectors and/or cassette of the invention that contains the nucleic acids sequence encoding the CAT-T of the invention, into packaging cell lines and of collecting the viral particles that are generated by the packaging lines are well known in the art. Nonviral vectors, in accordance with the invention, refer to all the physical and chemical systems except viral systems and generally include either chemical methods, such as cationic liposomes and polymers, or physical methods, such as gene gun, electroporation, particle bombardment, ultrasound utilization, and magnetofection. Efficiency of this system is less than viral systems in gene transduction, but their cost-effectiveness, availability, and more importantly reduced induction of immune system and no limitation in size of transgenic DNA compared with viral system have made them attractive also for gene delivery.

For example, physical methods applied for in vitro and in vivo gene delivery are based on making transient penetration in cell membrane by mechanical, electrical, ultrasonic, hydrodynamic, or laser-based energy so that DNA entrance into the targeted cells is facilitated.

In more specific embodiments, the vector may be a naked DNA vector. More specifically, such vector may be for example, a plasmid, minicircle or linear DNA. Naked DNA alone may facilitate transfer of a gene (2-19 kb) into skin, thymus, cardiac muscle, and especially skeletal muscle and liver cells when directly injected. It enables also long-term expression. Although naked DNA injection is a safe and simple method, its efficiency for gene delivery is quite low.

Minicircles are modified plasmid in which a bacterial origin of replication (ori) was removed, and therefore they cannot replicate in bacteria. Linear DNA or Doggybone™ are double-stranded, linear DNA construct that solely encodes an antigen expression cassette, comprising antigen, promoter, polyA tail and telomeric ends. It should be appreciated that all DNA vectors disclosed herein, may be also applicable for all nucleic acid molecules, vectors and/or cassettes used in the methods and compositions of the invention, as described herein. Still further, it must be appreciated that the invention further provides any vectors or vehicles that comprise any of the nucleic acid molecules, vectors and/or nucleic acid cassettes disclosed by the invention, as well as any host cell expressing the nucleic acid molecules, and/or nucleic acid cassettes disclosed by the invention.

A further aspect of the present disclosure relates to a gene editing system comprising:
First (i), at least one nucleic acids molecule as defined by the present disclosure, or any cassette, vector or vehicle comprising the at least one nucleic acid molecule; and
Second (ii), at least one gene editing component or a nucleic acid sequence encoding the gene editing component. In some specific embodiments, the at least one nucleic acids molecule of the gene editing system provided by the present disclosure encodes at least one CAR molecule comprising the following components: (i), at least one target-binding domain; wherein at least one of the target binding domain specifically recognizes and binds BCMA; and optionally (ii), at least one hinge and at least one transmembrane domain derived from the CD8α protein. It should be noted that the hinge region of the domain comprises the amino acid sequence as denoted by SEQ ID NO:9, and any fragments, derivatives and variants thereof; and (iii), at least one intracellular T cell signal transduction domain. More specifically, this domain comprises at least one domain of TNF receptor family member, and optionally, at least one domain of a TCR molecule.

As discussed herein, such system further comprises any gene editing component as discussed herein after, that enables and facilitates the insertion of the nucleic acid sequence that encodes any of the CAR molecules of the present disclosure, into a target site within the genome of any target cell.

In some embodiments, a gene editing component of the gene editing system disclosed herein, may be any one of a site-specific nuclease, a class switch recombination, a site specific integrase and a site-specific recombinase.

In some specific embodiments, a gene editing component useful in the systems of the present disclosure may be the CRISPR/Cas.

More specifically, in some embodiments, the CAR T encoding nucleic acid sequences (e.g., in a nucleic acid cassette) is inserted into the appropriate target genomic locus using a site-specific nuclease. The nuclease may be one of the following: CRISPR/Cas9/Cpf1/CTc(1/2/3), SpCas9, SaCas9, engeineerd CAS9, ZFN, TALEN, Homing endonuclease, Meganuclease, Mega-TALEN. The nuclease may be coded on a DNA vector such as a plasmid, a mini-circle or a viral vector. Alternatively, the mRNA coding for the nuclease may be delivered, or the nuclease may be delivered as a protein. A guide RNA may be provided or a DNA vector coding for a guide RNA. Integration catalyzed by a nuclease may utilize homologous arms flanking the DNA to be inserted or utilize recognition sites for the site-specific nuclease when such were coded preceding and or following the DNA to be inserted. Delivery of the nuclease or the vector coding for the nuclease can take place in vivo or ex vivo using autologous or allogeneic cells, as will be discussed herein after.

As specified above, in some embodiments, a nuclease useful for targeted insertion of the nucleic acid sequence encoding the desired CAR molecule disclosed herein may comprise at least one component of the CRISPR-Cas system. The Clustered Regularly Interspaced Short Palindromic Repeats (CRISPR) system is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

It should be understood that the present disclosure contemplates the use of any of the known CRISPR systems, particularly any of the CRISPR systems disclosed herein. The CRISPR-Cas system has evolved in prokaryotes to protect against phage attack and undesired plasmid replication by targeting foreign DNA or RNA. In bacterial immunity, the CRISPR-Cas system, targets DNA molecules based on short homologous DNA sequences, called spacers that have previously been extracted by the bacterium from the foreign pathogen sequence and inserted between repeats as a memory system. These spacers are transcribed and processed and this RNA, named crRNA or guide-RNA (gRNA), guides CRISPR-associated (Cas) proteins to matching (and/or complementary) sequences within the foreign DNA, called proto-spacers, which are subsequently cleaved. The spacers, or other suitable constructs or RNAs can be rationally designed and produced to target any DNA sequence. Moreover, this recognition element may be designed separately to recognize and target any desired target including outside of a bacterium.

In some specific embodiment, the CRISPR-Cas proteins used in the present disclosure may be of a CRISPR Class 2 system. In yet some further particular embodiments, such class 2 system may be any one of CRISPR type II, and type V systems. In certain embodiments, the Cas applicable in the present invention may be any Cas protein of the CRISPR type II system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein. It should be appreciated that any type II CRISPR-Cas systems may be applicable in the present invention, specifically, any one of type II-A or B. Thus, in yet some further and alternative embodiments, at least one cas gene used in the methods and systems of the invention may be at least one cas gene of type II CRISPR system (either typeII-A or typeII-B). In more particular embodiments, at least one cas gene of type II CRISPR system used by the methods and systems of the invention may be the cas9 gene.

According to such embodiments, the CRISPR-Cas proteins used in the systems of the invention is a CRISPR-associated endonuclease 9 (Cas9). Double-stranded DNA (dsDNA) cleavage by Cas9 is a hallmark of “type II CRISPR-Cas” immune systems. The CRISPR-associated protein Cas9 is an RNA-guided DNA endonuclease that uses RNA:DNA complementarity to a target site (proto-spacer). After recognition between Cas9 and the target sequence double stranded DNA (dsDNA) cleavage occur, creating the double strand breaks (DSBs).

CRISPR type II system as used herein requires the inclusion of two essential components: a “guide” RNA (gRNA) and a CRISPR-associated endonuclease (Cas9). The gRNA is an RNA molecule composed of a “scaffold” sequence necessary for Cas9-binding (also named tracrRNA) and about 20 nucleotide long “spacer” or “targeting” sequence, which defines the genomic target to be modified. Guide RNA (gRNA), as used herein refers to a synthetic fusion or alternatively, annealing of the endogenous tracrRNA with a targeting sequence (also named crRNA), providing both scaffolding/binding ability for Cas9 nuclease and targeting specificity. Also referred to as “single guide RNA” or “sgRNA” or as a specificity conferring nucleic acid (SCNA).

In yet some further particular embodiments, the class 2 system in accordance with the invention, may be a CRISPR type V system. In a more specific embodiment, the RNA guided DNA binding protein nuclease may be CRISPR-associated endonuclease X (CasX) system or CRISPR-associated endonuclease 14 (Cas14) system or CRISPR-associated endonuclease F (CasF, also known as Cas12j) system. The type V CRISPR-Cas systems are distinguished by a single RNA-guided RuvC domain-containing nuclease. As with type II CRISPR-Cas systems, CRISPR type V system as used herein requires the inclusion of two essential components: a gRNA and a CRISPR-associated endonuclease (CasX/Cas14/CasF). The gRNA is a short synthetic RNA composed of a “scaffold” sequence necessary for CasX/Cas14/CasF-binding and about 20 nucleotide long “spacer” or “targeting” sequence, which defines the genomic target to be modified.

It should be noted that the gRNA used herein may comprise between about 3 nucleotides to about 100 nucleotides, specifically, 3, 4, 5, 6, 7, 8, 9, 10, 15, 20, 25, 30, 35, 40, 45, 50, 55, 60, 65, 70, 75, 80, 85, 90, 95 100 or more. More specifically between about 10 nucleotides to 70 nucleotides or more.

It should be noted that any CRISPR/Cas proteins may be used by the gene editing system/s of the present disclosure, in some embodiments of the present disclosure, the endonuclease may be a Cas9, CasX, Cas12, Cas13, Cas14, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM10152, Rhodococcus erythropolis PR4, Rhodococcus jostii RFIA1, Rhodococcus opacus B4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bd1, Bifidobacterium longum DJO10A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei191, uncultured Termite group 1 bacterium phylotype Rs D17, Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS10565, Streptococcus gallolyticus UCN34 (uid46061), Streptococcus gordonii Challis subst CH1, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus, moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter eiseniae EFO1-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U 112, Francisella tularensis holarctica, Francisella tularensis FSC 198, Francisella tularensis, Francisella tularensis WY96-3418, or Treponema denticola (ATCC 35405).

In some embodiments, the CAR T encoding nucleic acid sequences (e.g. in a nucleic acid cassette) is inserted into the appropriate genomic locus using a site-specific recombinase/integrase. The recombinase/integrase may be one of the following: PhiC31, HK022, Cre, Flp, and more. The recombinase/integrase may be coded on a DNA vector such as a plasmid, a mini-circle or a viral vector. Alternatively, the mRNA coding for the recombinase/integrase may be delivered, or the recombinase/integrase may be delivered as a protein. Delivery of the nuclease or the vector coding for the recombinase/integrase can take place in vivo or ex vivo using autologous or allogeneic cells. A further aspect of the preset disclosure relates to a genetically engineered cell of the T cell lineage expressing at least one CAR molecule, or any population of cells comprising at least one genetically modified cell/s as disclosed herein. More specifically, in some embodiments, the CAR expressed by the engineered cells comprises the following components: First (i), at least one target-binding domain. It should be further noted at least one of the target binding domain specifically recognizes and binds BCMA. The second component (ii), is at least one hinge and at least one transmembrane domain derived from the CD8α protein. It should be noted that the hinge region of the domain comprises the amino acid sequence as denoted by SEQ ID NO:9, and any fragments, derivatives and variants thereof. The CAR molecule further comprises as a third component (iii), at least one intracellular T cell signal transduction domain. More specifically, this domain comprises at least one domain of TNF receptor family member, and optionally, at least one domain of a TCR molecule.

In some embodiments, the present disclosure provides any genetically engineered cell that express any of the CAR molecule/s defined by the present disclosure.

In some embodiments, the genetically engineered cell of the present disclosure may be any hematopoietic cell. In some embodiments, such cell may be any lymphocyte. In some further embodiments such cell may be any cell of the T lineage. In some specific embodiments, such cell is at least one T cell and/or at least one NK T cell.

As indicted in the present aspect, the present invention provides an engineered cell that may be any lymphocyte, specifically, any lymphocyte of the T lineage. “Lymphocytes” are mononuclear nonphagocytic leukocytes found in the blood, lymph, and lymphoid tissues. They are divided on the basis of ontogeny and function into two classes, B and T lymphocytes, responsible for humoral and cellular immunity, respectively. Most are smalllymphocytes 7-10 m in diameter with a round or slightly indented heterochromatic nucleus that almost fills the entire cell and a thin rim of basophilic cytoplasm that contains few granules. When “activated” by contact with antigen, small lymphocytes begin macromolecular synthesis, the cytoplasm enlarges until the cells are 10-30 m in diameter, and the nucleus becomes less completely heterochromatic; they are then referred to as large lymphocytes or lymphoblasts. These cells then proliferate and differentiate into B and T memory cells and into the various effector cell types: B cells into plasma cells and T cells into helper, cytotoxic, and suppressor cells.

As indicated by the present disclosure, the genetically engineered cells that express the CAR molecule disclosed herein, may be cells of the T lineage. A “T cell” or “T lymphocyte” as used herein is characterized by the presence of a T-cell receptor (TCR) on the cell surface. It should be noted that T-cells include helper T cells (“effector T cells” or “Th cells”), cytotoxic T cells (“Tc,” “CTL” or “killer T cell”), memory T cells, and regulatory T cells as well as Natural killer T cells, Mucosal associated invariants and Gamma delta T cells.

More specifically, Thymocytes are hematopoietic progenitor cells present in the thymus. Thymopoiesis is the process in the thymus by which thymocytes differentiate into mature T lymphocytes. The thymus provides an inductive environment, which allows for the development and selection of physiologically useful T cells. The processes of beta-selection, positive selection, and negative selection shape the population of thymocytes into a peripheral pool of T cells that are able to respond to foreign pathogens and are immunologically tolerant towards self-antigens.

Thymocytes are classified into a number of distinct maturational stages based on the expression of cell surface markers. The earliest thymocyte stage is the double negative (DN) stage (negative for both CD4 and CD8), which more recently has been better described as Lineage-negative, and which can be divided into four sub-stages. The next major stage is the double positive (DP) stage (positive for both CD4 and CD8). The final stage in maturation is the single positive (SP) stage (positive for either CD4 or CD8).

More specifically, the maturational stages of thymocytes may include the following substages: Double negative 1 (DN1) or ETP (Early T lineage Progenitor) is characterized by CD44+CD25-CD117+ defining surface markers, thymocytes are located in the cortex and proliferation, loss of B and myeloid potentials are observed; Double negative 2 (DN2) is characterized by CD44+CD25+CD117+ defining surface markers and thymocytes are located in the cortex; Double negative 3 (DN3) is characterized by CD44−CD25+ defining surface markers, thymocytes are located in the cortex and TCR-beta rearrangement and beta selection are observed; Double negative 4 (DN4) is characterized by CD44−CD25− defining surface markers and thymocytes are located in the cortex; Double positive is characterized by CD4+CD8+ defining surface markers, thymocytes are located in the cortex and TCR-alpha rearrangement, positive selection, negative selection are observed; Single positive is characterized by CD4+CD8− or CD4−CD8+ defining surface markers, thymocytes are located in the medulla and Negative selection is observed.

In human, circulating CD34+ hematopoietic stem cells (HSC) reside in bone marrow. They produce precursors of T lymphocytes, which seed the thymus (thus becoming thymocytes) and differentiate under influence of the Notch and its ligands. Early, double negative thymocytes express (and can be identified by) CD2, CD5 and CD7. Still during the double negative stage, CD34 expression stops and CD1 is expressed. Expression of both CD4 and CD8 makes them double positive and matures into either CD4+ or CD8+ cells. It should be appreciated that a cell of the T lineage as disclosed herein may be any of the thymocytes disclosed herein at any stage/substage and/or expressing any of the disclosed markers.

In some embodiments of the invention, the host cell is a T cell. For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4.sup.+/CD8.sup.+ double positive T cells, CD4.sup.+ helper T cells, e.g., Th.sub.1 and Th.sub.2 cells, CD8.sup.+ T cells (e.g., cytotoxic T cells), tumor infiltrating cells, memory T cells, naive T cells, and the like. The T cell may be a CD8.sup.+ T cell or a CD4.sup.+ T cell.

In an embodiment of the invention, the host cell is a natural killer (NK) cell. NK cells are a type of cytotoxic lymphocyte that plays a role in the innate immune system. NK cells are defined as large granular lymphocytes and constitute the third kind of cells differentiated from the common lymphoid progenitor which also gives rise to B and T lymphocytes. NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human).

Still further, in some alternative embodiments, the genetically engineered cells may be of the B lineage. Also provided by an embodiment of the invention is a population of cells comprising at least one host cell described herein, e.g., of the T lineage. The population of cells can be a heterogeneous population comprising the host cell comprising and/or genetically engineered by any of the nucleic acid sequences, cassettes, vectors and gene editing systems described herein, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector, cassette, ene editing system. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells that are genetically edited and/or comprising the nucleic acid sequences, cassettes and vectors as described herein.

The present disclosure provides cells, specifically, of the T lineage that were genetically engineered to express the CAR T molecules disclosed herein. It should be however noted that the present disclosure further encompasses any host cel comprising, transfected by, transformed by and/or engineered and/or edited by the nucleic acid sequence, cassette or vector disclosed herein. The term “host cell” includes a cell into which a heterologous (e.g., exogenous) nucleic acid or protein has been introduced. Persons of skill upon reading this disclosure will understand that such terms refer not only to the particular subject cell, but also is used to refer to the progeny of such a cell. Because certain modifications may occur in succeeding generations due to either mutation or environmental influences, such progeny may not, in fact, be identical to the parent cell, but are still included within the scope of the term “host cell”. As used herein, a cell has been “transformed” or “transfected” by exogenous or heterologous DNA, e.g., the nucleic acid molecule/s of the invention or any cassette, vector and/or gene editing system of the invention, when such DNA has been introduced inside the cell. The transforming DNA may be integrated (covalently linked) into the genome of the cell. With respect to the present invention, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. It should be appreciated that in some embodiments, the host cells of the invention may be any engineered T cells of the invention or any cell population comprising, at least in part, the T cells of the invention. Still further, the invention further encompasses any population of cells comprising at least 5%, 10%, 15%, 20%, 25%, 30%, 35%, 40%, 45%, 50%, 55%, 60%, 65%, 70%, 75%, 80%, 85%, 90%, 95%, 99.9% or more, specifically, 100%) specifically, 10%, 15%, 20%, 25%, 30%, 35%, 40%, 45%, 50%, 55%, 60%, 65%, 70%, 75%, 80%, 85%, 90%, 95%, 99.9% or more, specifically, 100% of the host cells, specifically, the genetically engineered T cells of the invention.

A further aspect of the present disclosure relates to a composition comprising at least one CAR molecule, any nucleic acid molecule comprising at least one nucleic acid sequence encoding said CAR molecule, or any, cassette, vector, vehicle or gene editing system comprising the nucleic acid molecule, any host cell expressing said CAR molecule, and/or any genetically engineered cell of the T lineage expressing said CAR or population of cells comprising at least one the genetically engineered cell of the T lineage. More specifically, such CAR molecule comprises the following components. First (i), at least one target-binding domain; wherein at least one of said target binding domain specifically recognizes and binds BCMA. The second component (ii), at least one hinge and/or at least one transmembrane domain derived from the CD8α protein. It should be noted that the hinge region of the domain comprises the amino acid sequence as denoted by SEQ ID NO:9, and any fragments, derivatives and variants thereof. The CAR molecule further comprises as a third component (iii), at least one intracellular T cell signal transduction domain. More specifically, this domain comprises at least one domain of TNF receptor family member, and optionally, at least one domain of a TCR molecule.

It should be noted that the composition of the present disclosure further comprises according to optional embodiments, at least one of pharmaceutically acceptable carrier/s, diluent/s, excipient/s and additive/s.

Still further, it should be understood that in some embodiments, the compositions of the present invention may comprise any of the CAR molecules as defined by the present disclosure. In some alternative or additional embodiments, the compositions disclosed herein may comprise any of the nucleic acid molecule/s as defined by the present disclosure. In yet some further alternative or additional embodiments, the compositions disclosed herein may comprise any of the gene editing system/s defined by the present disclosure. In yet some further alternative or additional embodiments, the compositions disclosed herein may comprise any of the cell/s or population of cells as defined by the present disclosure.

The compositions of the invention may comprise an effective amount of the nucleic acid molecules, and/or cassette thereof or of any vector thereof or of any cell comprising the same, or any CAR T molecule as described by the invention. The term “effective amount” relates to the amount of an active agent present in a composition, specifically, the nucleic acid molecules, vectors and/or cassette of the invention as described herein that is needed to provide a desired level of active agent in the bloodstream or at the site of action in an individual (e.g., the thymus or bone marrow) to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An “effective amount” of the genetically engineered CAR T cells disclosed herein, or any nucleic acid molecule/s of the invention or any cassette of the invention and gene editing systems thereof, can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual. Still further, administration and doses are determined by good medical practice of the attending physician and may depend on the age, sex, weight and general condition of the subject in need.

It should be appreciated that the effective amount as discussed herein is applicable for each and every embodiment of each and every aspect of the present disclosure, specifically, for any of the CAR molecule, CAR T cells expressing the CAR molecule of the present disclosure, and/or any nucleic acid sequence encoding the disclosed CAR molecule, any construct, or ene editing system comprising the same, any dosage forms thereof, dosage unit forms thereof, compositions, kits, uses and methods thereof.

In some embodiments, the active ingredient of the therapeutic composition, and/or dosage form disclosed herein, is T cells genetically engineered to express the HBI101 CAR molecule (also designated herein as H8BB CAR molecule) of the present disclosure. As shown in the clinical studies disclosed herein, various effective amounts of these cells were used. In some embodiments, the effective amount of HBI101 CAR T cells may range between about 50×10⁶ cells to about 1500×10⁶ cells, specifically, 50×10⁶, 60×10⁶, 70×10⁶, 80×10⁶, 90×10⁶, 100×10⁶, 110×10⁶, 120×10⁶, 130×10⁶, 140×10⁶, 150×10⁶, 160×10⁶, 170×10⁶, 180×10⁶, 190×10⁶, 200×10⁶, 210×10⁶, 220×10⁶, 230×10⁶, 240×10⁶, 250×10⁶, 260×10⁶, 270×10⁶, 280×10⁶, 290×10⁶, 300×10⁶, 310×10⁶, 320×10⁶, 330×10⁶, 340×10⁶, 350×10⁶, 360×10⁶, 370×10⁶, 380×10⁶, 390×10⁶, 400×10⁶, 410×10⁶, 420×10⁶, 430×10⁶, 440×10⁶, 450×10⁶, 460×10⁶, 470×10⁶, 480×10⁶, 490×10⁶, 500×10⁶, 510×10⁶, 520×10⁶, 530×10⁶, 540×10⁶, 550×10⁶, 600×10⁶, 650×10⁶, 700×10⁶, 750×10⁶, 800×10⁶, 850×10⁶, 900×10⁶, 950×10⁶, 1000×10⁶, 1050×10⁶, 1100×10⁶, 1150×10⁶, 1200×10⁶, 1250×10⁶, 1300×10⁶, 1350×10⁶, 1400×10⁶, 1450×10⁶, 1500×10⁶/HBI101 CART cells/per dose. More specifically, the amount and/or number of HBI101 CAR T cells may range between about 100×10⁶ to about 1000×10⁶, about 90×10⁶ to about 900×10⁶, about 150×10⁶ to about 800×10⁶. Still further, in some embodiments, an effective amount and/or number of cells is 800×10⁶-HBI101 CAR T cells, per dose. In yet some other embodiments, an effective amount and/or number of cells is 450×10⁶. HBI101 CAR T cells, per dose. Still further, in some embodiments. an effective amount and/or number of cells is 150×10⁶. HBI101 CAR T cells, per dose. Still further, in some embodiments, the effective amount and/or number of cells may range between about 0.5×10⁶ to about 50×10⁶ HBI101 CAR T cells/per Kg of body weight. Specifically, about 1 to about 40, about 2×10⁶ to about 30×10⁶, about 2×10⁶ to about 25×10⁶, about 2×10⁶ to about 20×10⁶, about 2×10⁶ to about 15×10⁶, about 2×10⁶ to about 14×10⁶, about 2×10⁶ to about 13×10⁶, about 2×10⁶ to about 12×10⁶, about 2×10⁶ to about 11×10⁶, about 2×10⁶ to about 10×10⁶ HBI101 CAR T cells/per Kg of body weight. In some specific and non-limiting embodiments, effective amount of the cells is 11×10⁶ HBI101 CAR T cells/per Kg of body weight. In some other specific and non-limiting embodiments, effective amount of the cells is 10×10⁶ HBI101 CAR T cells/per Kg of body weight. Still further, in some embodiments, the effective amount may be 12×10⁶ HBI101 CAR T cells/per Kg of body weight. Still further, as demonstrated by the clinical data disclosed herein, in some embodiments, a dose of 800×10⁶, provides a clear increase in efficacy, while not affecting the toxicity. More specifically, as shown by FIG. 34, about 58% of the patients showed a complete response, the OS is about 95% (at day 380 of the study), and about 70% PFS. These results showed improved efficacy specifically when compared to prior art CAR T derived from the C11D5-3 anti-BCMA antibody (e.g., in references [2], [5] and [10]), that use a lower dose of cells showing reduced efficacy. Thus, in some embodiments, an effective amount as used herein, refers to a daily dose of 800×10⁶.

The pharmaceutical compositions of the invention can be administered and dosed by the methods of the invention, in accordance with good medical practice, systemically, for example by parenteral, e.g., intrathymic, into the bone marrow and intravenous. It should be noted however that the invention may further encompass additional administration modes. In other examples, the pharmaceutical composition can be introduced to a site by any suitable route including intraperitoneal, subcutaneous, transcutaneous, topical, intramuscular, intraarticular, subconjunctival, or mucosal, e.g., oral, intranasal, or intraocular administration.

Local administration to the area in need of treatment may be achieved by, for example, by local infusion during surgery, topical application, direct injection into the specific organ (bone marrow, spleen, lymph nodes), etc. More specifically, the compositions used in any of the methods of the invention, described herein, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the CAR T, nucleic acid molecule/s of the invention or any cassette/s thereof, with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations. It should be understood that in addition to the ingredients particularly mentioned above, the formulations may also include other agents conventional in the art having regard to the type of formulation in question. Still further, pharmaceutical preparations are compositions that include one or more nucleic acid molecules, vectors and/or cassette and/or cells of the present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle”, when referred to the compositions in the present aspect, refers to a diluent, adjuvant, excipient, or carrier with which a compound of the invention is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the nucleic acid molecule/s encoding the CARs of the invention or any engineered cells of the T-lineage, and systems of the invention, can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity, or it may be formulated for sustained release.

In numerous embodiments, the compositions of the present invention may be administered in a form of combination therapy, i.e. in combination with one or more additional therapeutic agents. Combination therapy may include administration of a single pharmaceutical dosage formulation comprising at least one composition of the invention and additional therapeutics agent(s); as well as administration of at least one composition of the invention and one or more additional agent(s) in its own separate pharmaceutical dosage formulation. Further, where separate dosage formulations are used, compositions of the invention and one or more additional agents can be administered concurrently or at separately staggered times, i.e. sequentially. Still further, the concurrent or separate administrations may be carried out by the same or different administration routes. Thus, in some further embodiments, the CAR T molecules, nucleic acid sequences, cassettes, systems and cells of the present disclosure may be applicable in boosting the immune response of a subject suffering from an immune-related disorder, specifically, any disorder involving B Cells, or B cell malignancies, and may be used in combined treatment with any therapeutic agent, for example, a chemotherapeutic agent.

As used herein, a “chemotherapeutic agent” or “chemotherapeutic drug” (also termed chemotherapy) as used herein refers to a drug treatment intended for eliminating or destructing (killing) cancer cells or cells of any other proliferative disorder. The mechanism underlying the activity of some chemotherapeutic drugs is based on destructing rapidly dividing cells, as many cancer cells grow and multiply more rapidly than normal cells. As a result of their mode of activity, chemotherapeutic agents also harm cells that rapidly divide under normal circumstances, for example bone marrow cells, digestive tract cells, and hair follicles. Insulting or damaging normal cells result in the common side-effects of chemotherapy: myelosuppression (decreased production of blood cells, hence also immuno-suppression), mucositis (inflammation of the lining of the digestive tract), and alopecia (hair loss).

Various different types of chemotherapeutic drugs are available. A chemotherapeutic drug may be used alone or in combination with another chemotherapeutic drug or with other forms of cancer therapy, in addition to the CAR T of the present disclosure, for example, other biological drugs (antibodies, ligands, receptors), radiation therapy or surgery.

Chemotherapeutic drugs affect cell division or DNA synthesis and function and can be generally classified into several groups, based on their structure or biological function. More specifically, chemotherapeutic agents that are classified as alkylating agents, anti-metabolites, anthracyclines, plant alkaloids, topoisomerase inhibitors, and other anti-tumor agents such as DNA-alkylating agents, anti-tumor antibiotic agents, tubulin stabilizing agents, tubulin destabilizing agents, hormone antagonist agents, protein kinase inhibitors, HMG-CoA inhibitors, CDK inhibitors, cyclin inhibitors, caspase inhibitors, metalloproteinase inhibitors, antisense nucleic acids, triple-helix DNAs, nucleic acids aptamers, and molecularly-modified viral, bacterial or exotoxic agents. It should be appreciated that any combination therapy disclosed herein, using any of the indicated compounds with the CAR T, DNA cassettes, systems and cells of the present disclosure, together with any of the therapeutic agents discussed above, is encompassed by the present disclosure.

Still further, in some embodiments, the construct encoding the disclosed CAR molecule of the present disclosure, also referred to herein as HBI0101 (prepared in a clinical grade), may be generated by transiently transfecting Phoenix-ECO cells (ATCC) with the plasmid encoding the gamma-retroviral vector MSGV1-HBI0101 using JetPrime reagent (Tamar) and subsequently transducing PG13 cells (ATCC) with HBI0101 Phoenix-ECO cell-free vector supernatants. PG13 transduced population was subsequently sub-cloned by limiting dilution, and the PG13-HBI0101 expanded to generate a seed bank. The certified PG13 seed bank was sent to the Indiana University Vector Production Facility (IU-VPF) in Indianapolis that has generated a master cell bank (MCB) and a GIP-certified HBI0101 clinical grade retroviral supernatant for the transduction of MM patients' autologous T-cells.

Still further, for ex vivo transduction of the cells, peripheral blood mononuclear cells from subjects (e.g., AL and/or multiple myeloma patients) blood were isolated and CD3-activated peripheral blood mononuclear cells were used for further production. Blood was collected from patients with AL or/and multiple myeloma (0253-20-HMO) and processed, for example, using a Ficoll gradient to isolate peripheral blood mononuclear cells (PBMC). PBMCs were suspended at a concentration of 1×10⁶ cells per mL in T-cell medium (e.g., TCM), containing AIM-V (e.g., Gibco) supplemented with 5% human serum (e.g., Valley), 1% Glutamax (e.g., Gibco). IL2 (e.g., 300 IU/mL; e.g., Proleukin, Novartis) and anti-CD3 monoclonal antibody OKT-3 (e.g., 50 ng/mL; Miltenyi Biotech) were added to the TCM for 2 days of culture. Tissue culture non-treated 24-well plates were coated with 10 mg/mL RectroNectin (e.g., R/N; Takara) in PBS (e.g., Lonza) overnight at 4° C., followed by 30 minutes blocking with 2.5% human albumin in PBS, then washed. Retroviral supernatant was thawed, diluted 1:20 with TCM, added to wells, and centrifuged at 2,000 g for 2 hours at 32° C. The supernatant was then aspirated and 0.5×10⁶ CD3-activated PBMCs/mL were seeded into each well in TCM with 300 IU/mL IL2, centrifuged for 10 minutes at 1,000 g, and incubated at 37° C. overnight. Activated but non-transduced (NT) cells were generated and used as T-cell controls. Transduction efficacy was determined at days 6 and 10 of the culture via flow cytometry, by labeling BCMA CAR T cells with the human recombinant BCMA protein (e.g., Active; ACRO).

Still further, in some embodiments, For clinical grade production of HBI0101 cells, the same protocol as described above for the production of the cells for ex vivo applications, may be used. Modifications as to the source of the starting material, the use of clinical grade medium and reagents, and production under GMP conditions, were made to generate CART cells suitable for the clinic.

More specifically, in some embodiments, leucocytes are collected at day −10 by leukapheresis, using the Spectra Optia apheresis system. Cells are separated to peripheral mono-nuclear cells (e.g., PBMCS) and T-cell stimulated using anti-CD3 and IL-2. At day −8, stimulated T cells are transduced with 1/25 or 1/50 diluted HBI0101 retroviral supernatant overnight. Then, cells are seeded into GRex100 devices filled with AIM-V medium (e.g., Gibco) supplemented with 5% human AB serum (e.g., Access Cell Culture or Valley), 1% Glutamax (Gibco) and 300 IU/mL IL-2 for seven days of expansion. In some embodiments, medium and IL-2 replenishment is performed every 2-3 days. At the day of patient's infusion, cells are washed 3 times with saline with 1% human albumin (Kedrion), and then formulated into the final drug product (DP) at the concentration of 15×10⁶ CART cells/mL in saline with 2.5% human albumin. DP infusion volume varied according to cell doses.

Still further, in some embodiments, quality control testing of in-process (IP) and end-of-product (EOP) HBI0101 cells are performed along the manufacturing process. In some embodiments, quality control testing may include: i) determination of the percent of transduction, assessed by flow cytometry using BCMA-FITC recombinant protein (e.g., ACROBiosystems), and performed at days −7, −2 and 0; ii) in-vitro efficacy of CART cells, assessed by the release of interferon-γ by ELISA (R&D) following stimulation with myeloma cell line, and performed at day −2; iii) determination of the vector copy number (CPN) by real-time (RT) PCR of the transduced cells' genomic DNA at day −2; iv) the absence of replication competent retrovirus (RCR) at day −2 is confirmed by PCR analysis of the transduced cells' genomic DNA using GALV primers set, as detailed in 3; v) the characterization of the different cellular subsets was performed at day 0 by flow cytometry, using antibodies mixture as follows: anti-CD3 (e.g., Beckman Coulter), anti-CD4 (e.g., Biolegend), anti-CD8 (e.g., BD), anti-CD56 (e.g., Biolegend), anti-CD19 (e.g., Biolegend), and anti-CD14 (e.g., Beckman Coulter); vi) pH of the DP was assessed at day 0. All the tested parameters represent release criteria of the final DP, which specifications are further detailed in Table 5. In addition, and in compliance with ANNEX 1 guidelines for Advanced Therapy Medicinal Products (ATMPs), IP and EOP HBI0101 cells are tested for sterility according to the timeline detailed in FIG. 12B. Sterility testing were performed by an outsourced GMP-accredited institution (HyLabs).

A further aspect of the present disclosure relates to a method for treating, preventing, ameliorating, inhibiting or delaying the onset of an immune-related disorder in a mammalian subject. In some embodiments, the method comprises the step of administering to the subject an effective amount of at least one of:
(a) at least one nucleic acid molecule encoding least one CAR molecule; (b) at least one cassette, vector vehicle or gene editing system comprising the nucleic acid molecule of (a); (c) at least one cell (specifically, cell of the T linage) expressing said CAR, or a population of such cells; and (d) a composition comprising at least one of (a), (b) and (c). More specifically, in some embodiments of the disclosed methods, such e CAR molecule comprises the following components. First (i), at least one target-binding domain; wherein at least one of said target binding domain specifically recognizes and binds BCMA; second (ii), at least one hinge and at least one transmembrane domain derived from the CD8α protein. It should be noted that the hinge region of the domain comprises the amino acid sequence as denoted by SEQ ID NO:9, and any fragments, derivatives and variants thereof. The CAR molecule further comprises as a third component (iii), at least one intracellular T cell signal transduction domain. More specifically, this domain comprises at least one domain of TNF receptor family member, and optionally, at least one domain of a TCR molecule.

In some embodiments, the at least one target binding domain of the CAR molecule of the disclosed method comprises: (i) at least one target-recognition element; and/or (ii) at least one adaptor component that recognizes and binds at least one tagged target-recognition element.

In some embodiments, such adaptor component may comprise at least one moiety that specifically recognizes and binds at least one tag of the tagged target-recognition element.

In some further embodiments, the target-recognition domain of the CAR molecule of the disclosed methods comprises at least one antibody or any antigen-binding fragment/s, portion/s or chimera/s thereof, specific for the target BCMA.

In yet some further embodiments, the antigen-binding fragment/s, portion/s or chimera/s of the antibody used as the target-recognition domain of the CAR molecule of the disclosed methods comprises at least one of a scFv, and/or a nanobody.

In more specific embodiments, the antibody used as the target-recognition domain of the CAR molecule of the disclosed methods specifically recognizes and binds the BCMA protein. In more specific embodiments, the antibody comprises an immuno globulin HC comprising the amino acid sequence as denoted by SEQ ID NO: 3, and any derivatives and variants thereof, and an immunoglobulin LC comprising the amino acid sequence as denoted by SEQ ID NO: 5, and any derivatives and variants thereof.

In more specific embodiments, the least one target-binding domain of the CAR used by the methods of the present disclosure comprises the amino acid sequence as denoted by SEQ ID NO: 11, and any derivatives and variants thereof.

Still further, in some embodiments, the hinge and transmembrane domain of the CAR used by the methods of the present disclosure, comprises the amino acid sequence as denoted by SEQ ID NO: 6, and any derivatives and variants thereof.

In yet some further embodiments, the at least one intracellular T cell signal transduction domain of the CAR used by the methods of the present disclosure, comprises at least one TNF receptor family member. In some embodiments, such TNF receptor family member is the 4-1BB. Thus, the CAR molecule used by the methods of the present disclosure comprise at least one intracellular T cell signal transduction domain derived from the 4-1BB protein. In yet some further embodiments, the CAR molecule used by the methods of the present disclosure comprise at least one intracellular T cell signal transduction domain derived from a TCR molecule. More specifically, the CAR molecule used by the methods of the present disclosure comprise at least one intracellular T cell signal transduction domain derived from the CD3 zeta chain.

In some particular embodiments, the at least one intracellular T cell signal transduction domain of the CAR molecule used by the methods of the present disclosure comprises the amino acid sequence as denoted by SEQ ID NO: 12, and any derivatives and variants thereof.

In yet some further specific and non-limiting embodiments, the CAR molecule used by the methods of the present disclosure CAR comprises the amino acid sequence as denoted by SEQ ID NO: 1, or any variants and derivatives thereof, for example, the CAR T molecule of SEQ IS NO: 40 that does not contain the leader sequence.

Still further, in some embodiments, of the disclosed methods, the expression of the CAR molecule by at least one cell of the T lineage of the subject treated by the method of the invention results in at least one of the following alternative or additional effects.

In some alternative or additional embodiments, the expression of the CAR molecule by at least one cell of the T lineage of the subject treated by the method of the invention results in (i), increased specificity to the target. Particularly, increased specificity to any cells that express the target, BCMA. In yet some further additional or alternative embodiments, the expression of the CAR molecule by at least one cell of the T lineage of the subject treated by the method of the invention results in (ii), reduced tonic signaling. Still further, in some additional or alternative embodiments, the expression of the CAR molecule by at least one cell of the T lineage of the subject treated by the method of the invention results in (iii), reduced off-target activation. In some further additional or alternative embodiments, the expression of the CAR molecule by at least one cell of the T lineage of the subject treated by the method of the invention results in (iv), increased expression of activation markers in response to a specific stimulation. According to some embodiments, such activation markers may be for example, at least one of CD137, CD25, CD69. It should be further understood that specific stimulation as used herein, is the presence of BCMA-positive target cells. Still further in some additional or alternative embodiments, the expression of the CAR molecule by at least one cell of the T lineage of the subject treated by the method of the invention results in (v), reduced expression of exhaustion markers in response to a specific stimulation. More specifically, in some embodiments, the exhaustion markers may be at least one of PD-1, LAG-3, TIM3, TIGIT. In yet some further alternative and/or additional embodiments, the expression of the CAR molecule by at least one cell of the T lineage of the subject treated by the method of the invention results in (vi), increased survival of the treated subject. Still further, in some additional and/or alternative embodiments, the expression of the CAR molecule by at least one cell of the T lineage of the subject treated by the method of the invention results in (vii), reduced relapse rate in the treated subject. Still further, in some specific alternative and/or additional embodiments, the expression of the CAR molecule by at least one cell of the T lineage of the subject treated by the metho of the invention results in (viii) a long-term effect in the treated subject. More specifically, in some embodiments, the disclosed CAR molecules and CAR T cells thereof, and any compositions, systems and methods thereof, lead to increased survival of the treated subjects. Increase survival, as used herein, refers herein to any increase in either the overall survival, progression free survival or disease-free survival. Still further, in some embodiments, Overall Survival (also referred to herein as OS) is the length of time from either the date of diagnosis or the start of treatment for a disease, such as cancer, that patients diagnosed with the disease are still alive. In a clinical trial, measuring the overall survival is one way to see how well a new treatment works. In yet some further embodiments, Progression free survival (also referred to herein as PFS), is the length of time during and after the treatment of a disease, such as cancer, that a patient lives with the disease but it does not get worse. In a clinical trial, measuring the progression-free survival is one way to see how well a new treatment works. In yet some further embodiments, the disclosed the disclosed CAR molecules and CAR T cells thereof, and any compositions, systems and methods thereof, lead to increased DFS. Disease free survival (also referred to herein as DFS, relapse-free survival, and RFS). In cancer, the length of time after primary treatment for a cancer ends that the patient survives without any signs or symptoms of that cancer. In a clinical trial, measuring the disease-free survival is one way to see how well a new treatment works.

In yet some further embodiments, the disclosed CAR molecules and CAR T cells thereof, and any compositions, systems and methods thereof, lead to reduced relapse rate. The term “relapse”, as used herein, relates to the re-occurrence of a condition, disease or disorder that affected a person in the past, or any signs and symptoms of the disease after a period of improvement. For example, the term relates to the re-occurrence of a disease being treated with any therapeutic compound, e.g., the HBI101 CAR T cells of the present disclosure.

In some embodiments, reduced relapse rate is determined according to the International Myeloma Working Group(IMWG). More specifically, a clinical relapse of multiple myeloma requires one or more of the following: Development of new soft tissue plasmacytomas or bone lesions; definite increase in the size of existing plasmacytomas or bone lesions. A definite increase is defined as a 50% (and at least 1 cm) increase as measured serially by the sum of the products of the cross-diameters of the measurable lesion, Hypercalcemia (>11.5 mg/dL) [2.65 mmol/L], Decrease in haemoglobin of >2 g/dL [1.25 mmol/L], Rise in serum creatinine by 2 mg/dL or more [177 mmol/L or more].

Still further, in some embodiments, the disclosed CAR molecules and CAR T cells thereof, and any compositions, systems and methods thereof, provide a long term effect as demonstrated by the Examples. More specifically, in some embodiments, Long terms effects refer to higher proliferative capacity and persistence long-term survival benefits to T cells, including outgrowth of central memory T cells (Tcm), significantly enhanced respiratory capacity, increased fatty acid oxidation and enhanced mitochondrial biogenesis. T-cell memory is a critical component of immune responses. Following the antigen-driven expansion and the death of effector T cells after antigen clearance, some of the remaining T cells differentiate into memory T cells of two different types: central memory (Tcm) (identified as CD45RO+ CCR7+ cells) and effector memory T cells (Tem) (identified as CD45RO+ CCR7− cells). The first ones are located in lymphoid organs and bone marrow and have a high proliferative potential whereas the second ones stay in peripheral tissues in a preactivated form that enables them with immediate action on pathogen recognition. Naive and memory T cells rely primarily on the mitochondrial oxidation of free fatty acids for development and persistence. In contrast, activated effector T cells shift to glycolysis or concurrently upregulate oxidative phosphorylation and aerobic glycolysis to fulfill the metabolic demands of proliferation.

In some embodiments, the therapeutic methods of the present disclosure may use any of the CAR molecules as defined by the present disclosure. In some alternative or additional embodiments, the methods of the present disclosure may use any of the nucleic acid molecule/s as defined by the present disclosure. In yet some further alternative or additional embodiments, the methods of the present disclosure may use any of the gene editing system/s defined by the present disclosure. In yet some further alternative or additional embodiments, the methods of the present disclosure may use any of the cell/s or population of cells as defined by the present disclosure. In yet some further alternative or additional embodiments, the methods of the present disclosure may use any of the compositions defined by the present disclosure.

As indicated herein, the present disclosure provides method allowing in vivo as well as ex-vivo or in vitro genetic engineering of cells of the T lineage to express the CAR T molecules of the present invention. In case the engineering of the cells is performed ex vivo or in vitro, the engineered cells are transferred back to the subject, by adoptive transfer.

The term “adoptive transfer” as herein defined applies to all the therapies that consist of the transfer of components of the immune system, specifically cells that are already capable of mounting a specific immune response. In such option, the targeted insertion of the nucleic acid sequence encoding the CAR Ts disclosed herein, is performed in cells of an autologous or allogeneic source, that are then administered to the subject, specifically, by adoptive transfer.

In some embodiments, the cells that express, comprise, transduced or transfected with the nucleic acid molecule/s of the invention or any cassette provided by the invention may be cells of an autologous source. The term “autologous” when relating to the source of cells, refers to cells derived or transferred from the same subject that is to be treated by the methods of the invention. The term “allogenic” when relating to the source of cells, refers to cells derived or transferred from a different subject, referred to herein as a donor, of the same species.

In some embodiments of the therapeutic methods disclosed herein, the subject is administered with at least one cell of the T lineage expressing said CAR molecule, and/or genetically engineered with the at least one nucleic acid cassette or any vector or vehicle comprising the cassette, or with a population of the cells that comprise cells of the T lineage that express the CAR molecule of the present disclosure. According to such embodiments, the genetic engineering of the cells is performed ex vivo or in vitro. In yet some further embodiments, such cells are of an autologous or allogeneic source.

In yet some alternative embodiments, the genetic engineering of the cells of the T lineage is performed in vivo in the treated subject. In such case, the subject treated by the therapeutic methods disclosed herein is administered with a nucleic acid molecule comprising at least one nucleic acid sequence encoding the CAR T molecule of the present disclosure, or any cassette, vector or gene editing system comprising the same. As indicated above, the genetic editing of the cells of the T lineage to express the CAR molecules disclosed herein, is performed in vivo, in the treated subject. In some further embodiments an appropriate vector useful in such methods is any one of a viral vector, a non-viral vector and a naked DNA vector.

It should be understood that any of the vectors (e.g., viral vectors) disclosed herein in connection with other aspects of the preset disclosure, may be applicable also for genetic engineering of the cells of the T lineage, either ex vivo, in vitro, or alternatively, in vivo in the treated subject.

Still further, as discussed herein, in some embodiments of the therapeutic methods of the present disclosure insertion of the nucleic acids sequence that encodes the CAR into the genome of a cell of the T linage in the subject, is mediated by a site-specific nuclease. Such nuclease is at least one PEN. It should be understood that the use of PEN as discussed herein, may be applicable for both, an in vivo or ex vivo/in vitro introduction of the CAR encoding nucleic acid sequence, to the T cell genome. In yet some further specific embodiments, the PEN comprises at least one CRISPR/Cas protein system. Thus, in case the genetic engineering of the T cell is performed in vivo, according to such embodiments, the method further comprises the step of administering to the treated subject at least one of:
(a) at least one CRISPR/cas protein, or any nucleic acid molecule encoding said Cas protein; and
(b) at least one nucleic acid sequence comprising at least one gRNA that targets the insertion of the nucleic acid sequence encoding said CAR into a genomic sequence, or any nucleic acid sequence encoding the gRNA; or any kit, composition or vehicle comprising at least one of (a) and (b). it should be understood however, that where the genetic engineering of the cells of the T lineage is performed in vitro/ex vivo, and the subject is administered with the genetically engineered cells that express the CAR molecule of the present disclosure, the editing of this cells (either autologous or allogeneic cells), is performed by contacting the cells with the at least one CRISPR/cas protein and the gRNA as discussed above.

Still further, in some embodiments, the therapeutic methods of the present disclosure are applicable for any pathologic disorder, specifically, a disorder associated with expression of the BCMA protein in cells of the B lineage. In yet some further embodiments, the therapeutic methods may be applicable for any disorder associated with modulated expression, stability and/or activity of the BCMA protein. In some specific embodiments, the disclosed methods are applicable for disorders associated with, and/or characterized by overexpression of the BCMA protein.

Still further, in some embodiments, such disorder is at least one of: at least one plasma-cell pathology, at least one proliferative disorder, at least one deposition disorder and/or at least one autoimmune disease, or any B cell-mediated or associated disorder.

In some embodiments, the disclosed methods are applicable for treating at least one plasma-cell pathology. Plasma-cell pathology refers herein to any disorder which involves plasma cells. For example, multiple myeloma, myelomatosis and medullary plasmacytoma are bone marrow-based, malignant disorders of postgerminal center B-cells that is characterized by a clonal proliferation of plasma cells, with associated serum and/or urine monoclonal proteins.

In some embodiments, the plasm-cell pathology may comprise any one of Multiple myeloma (MM), amyloidosis (AL), Monoclonal gammopathy of undetermined significance (MGUS), Plasmacytoma (PL) and/or Waldenstrom macroglobulinemia (WDS).

Still further, Monoclonal gammopathy of undetermined significance (MGUS), as used herein, is an asymptomatic preneoplastic plasma cell disorder that is characterized by serum M-protein less than 30 g/L, bone marrow clonal plasma cells less than 10 percent, absence of plasma cell myeloma-related end-organ damage (hypercalcemia, renal insufficiency).

In some embodiments, Plasmacytoma (PL), is a plasma cell dyscrasia in which a plasma cell tumour grows within soft tissue or within the axial skeleton.

Waldenström macroglobulinemia (WDS), as used herein, is a neoplastic disorder affecting lymphoplasmacytoid cells and plasma cells. It is characterized by having high levels of a circulating antibody, immunoglobulin M (IgM), which is made and secreted by the cells involved in the disease. WDS is an “indolent lymphoma” (characterized with slow growth and spread) and a type of lymphoproliferative disease which shares clinical characteristics with the indolent non-Hodgkin lymphomas. It is commonly classified as a form of plasma cell dyscrasia, similar to other plasma cell dyscrasias that, for example, lead to multiple myeloma.

Multiple myeloma (MM) and amyloidosis (AL), will be described in more detail herein after.

As indicated above, the therapeutic methods of the present disclosure may be applicable for any disorder that is associated with the expression (e.g., modulated expression, specifically, overexpression) of the BCMA protein. Examples for such disorders include neoplastic disorders of B cells as disclosed above. However, it should be appreciated that in some embodiments, the disorder may be any proliferative disorder, or any neoplastic disorder. The methods of the present disclosure may be applicable in some embodiments for any neoplasms, either benign neoplasms, in situ neoplasms, or malignant neoplasms. As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods, compositions and kits of the present invention may be applicable for a patient suffering from any one of non-solid and solid tumors.

Malignancy, as contemplated in the present invention may be any one of lymphomas, leukemia, myeloma, carcinomas, melanomas and sarcomas. Therefore, in some embodiments any of the methods of the invention (provided that they involve, directly or indirectly, B cells and/or expression of BCMA), systems and compositions disclosed herein, may be applicable for any of the malignancies disclosed by the present disclosure.

More specifically, myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Still further, carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes.

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

It should be understood that the CAR molecules, nucleic acid molecules, cells, gene editing system/s, compositions and methods of the present disclosure are applicable for any type and/or stage and/or grade of any of the malignant disorders discussed herein or any metastasis thereof. Still further, it must be appreciated that the methods, compositions and systems of the invention may be applicable for invasive as well as non-invasive cancers. When referring to “non-invasive” cancer it should be noted as a cancer that do not grow into or invade normal tissues within or beyond the primary location. When referring to “invasive cancers” it should be noted as cancer that invades and grows in normal, healthy adjacent tissues.

Still further, in some embodiments, the methods, compositions and systems of the present disclosure are applicable for any type and/or stage and/or grade of any metastasis, metastatic cancer or status of any of the cancerous conditions disclosed herein.

As used herein the term “metastatic cancer” or “metastatic status” refers to a cancer that has spread from the place where it first started (primary cancer) to another place in the body. A tumor formed by metastatic cancer cells originated from primary tumors or other metastatic tumors, that spread using the blood and/or lymph systems, is referred to herein as a metastatic tumor or a metastasis. Thus, in some embodiments, malignancies that may find utility in the present invention can comprise but are not limited to hematological malignancies (including lymphoma, leukemia, myeloproliferative disorders, Acute lymphoblastic leukemia; Acute myeloid leukemia), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extrahepatic bile ducts, ampulla of Vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma, Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

As indicated above, the CAR molecules of the preset disclosure and any cells, specifically, cells of the T lineage genetically engineered to express the CAR molecules provided by the preset disclosure, may be applicable for any disorder that involves B cell.

Thus, in some specific embodiments, the methods of the present disclosure are applicable for treating proliferative disorder, specifically, any B cell malignancy.

In some embodiments, B cell malignancies include myeloma, specifically, multiple myeloma, as well as any type of lymphoma, including non-Hodgkin lymphomas as well as Hodgkin lymphomas.

More specifically, B-cell lymphomas make up most of the non-Hodgkin lymphomas (NHL). The methods of the present disclosure are therefore applicable for any type of lymphoma, specifically affecting B lymphocytes. The most common types of B-cell lymphomas applicable in the present disclosure, include, but are not limited to Diffuse large B-cell lymphoma (DLBCL), as well as to any subtype thereof (primary mediastinal B-cell lymphoma), Follicular lymphoma, diffuse large B-cell lymphoma, Chronic lymphocytic leukemia (CLL), small lymphocytic lymphoma (SLL), Mantle cell lymphoma (MCL), Marginal zone lymphomas, Extranodal marginal zone B-cell lymphoma, also known as mucosa-associated lymphoid tissue (MALT) lymphoma, Nodal marginal zone B-cell lymphoma, Splenic marginal zone B-cell lymphoma, Burkitt lymphoma, Lymphoplasmacytic lymphoma (Waldenstrom macroglobulinemia), Hairy cell leukemia (also called Chronic Lymphocytic Leukemia), Primary central nervous system (CNS) lymphoma, and Primary intraocular lymphoma (lymphoma of the eye).

Still further, in some embodiments, B cell-mediated disorder applicable in the present disclosure may be Hodgkin's lymphoma. As used herein, Hodgkin's lymphoma (formerly known as Hodgkin's disease) is a cancer of the immune system that is marked by the presence of a multinucleated cell type called Reed-Sternberg cells. The two major types of Hodgkin's lymphoma include classical Hodgkin's lymphoma and nodular lymphocyte-predominant Hodgkin's lymphoma and are both treatable by the compositions and methods disclosed herein.

In some embodiments, the therapeutic methods of the present disclosure may be applicable for a B cell malignancy such as multiple myeloma (MM) and any related conditions.

Multiple myeloma (MM), also known as plasma cell myeloma and simple myeloma, is a cancer of plasma cells, a type of white blood cell that normally produces antibodies. Often, no symptoms are noticed initially. As it progresses, bone pain, bleeding, frequent infections, and anemia may occur. Complications may include amyloidosis. The cause of multiple myeloma is unknown. Risk factors include obesity, radiation exposure, family history, and certain chemicals. Multiple myeloma may develop from monoclonal gammopathy of undetermined significance that progresses to smoldering myeloma. The abnormal plasma cells produce abnormal antibodies, which can cause kidney problems and overly thick blood. The plasma cells can also form a mass in the bone marrow or soft tissue. When only one tumor is present, it is called a plasmacytoma; more than one is called multiple myeloma. Multiple myeloma is diagnosed based on blood or urine tests finding abnormal antibodies, bone marrow biopsy finding cancerous plasma cells, and medical imaging finding bone lesions. Another common finding is high blood calcium levels. Because many organs can be affected by myeloma, the symptoms and signs vary greatly. The common symptoms of multiple myeloma are indicated as CRAB: C=calcium (elevated), R=renal failure, A=anemia, B=bone lesions. Myeloma has many other possible symptoms, including opportunistic infections (e.g., pneumonia) and weight loss. Multiple myeloma is considered treatable, but generally incurable. Monoclonal gammopathy of undetermined significance (MGUS) increases the risk of developing multiple myeloma. MGUS transforms to multiple myeloma at the rate of 1% to 2% per year, and almost all cases of multiple myeloma are preceded by MGUS.

Smoldering multiple myeloma increases the risk of developing multiple myeloma. Individuals diagnosed with this premalignant disorder develop multiple myeloma at a rate of 10% per year for the first 5 years, 3% per year for the next 5 years, and then 1% per year. Obesity is related to multiple myeloma with each increase of body mass index by five increasing the risk by 11%. Studies have reported a familial predisposition to myeloma. Hyperphosphorylation of a number of proteins, the paratarg proteins, a tendency that is inherited in an autosomal dominant manner, appears a common mechanism in these families. This tendency is more common in African-American with myeloma and may contribute to the higher rates of myeloma in this group. Rarely, Epstein-Barr virus (EBV) is associated with multiple myeloma, particularly in individuals who have an immunodeficiency due to e.g. HIV/AIDS, organ transplantation, or a chronic inflammatory condition such as rheumatoid arthritis. EBV-positive multiple myeloma is classified by the World Health Organization as one form of the Epstein-Barr virus-associated lymphoproliferative diseases and termed Epstein-Barr virus-associated plasma cell myeloma. EBV-positive disease is more common in the plasmacytoma rather than multiple myeloma form of plasma cell cancer. Tissues involved in EBV+ disease typically show foci of EBV+ cells with the appearance of rapidly proliferating immature or poorly differentiated plasma cells. The cells express products of EBV genes such as EBER1 and EBER2. While the EBV contributes to the development and/or progression of most Epstein-Barr virus-associated lymphoproliferative diseases, its role in multiple myeloma is not known. However, people who are EBV-positive with localized plasmacytoma(s) are more likely to progress to multiple myeloma compared to people with EBV-negative plasmacytoma(s). This suggest that EBV may have a role in the progression of plasmacytomas to systemic multiple myeloma. It should be thus understood that the methods of the present disclosure may be applicable for any type or stage of MM or any stage, background, source or type as disclosed herein.

In some particular embodiments, the methods of the invention are applicable to protein misfolding disorder or deposition disorder, also named proteopathy. The present disclosure provides in some embodiments thereof, therapeutic methods applicable for subjects suffering from any proteopathy, specifically, amyloidosis.

Proteopathy refers to a class of diseases in which certain proteins become structurally abnormal, and thereby disrupt the function of cells, tissues and organs of the body. Often the proteins fail to fold into their normal configuration; in this misfolded state, the proteins can become toxic in some way (a gain of toxic function) or they can lose their normal function. The proteopathies (also known as proteinopathies, protein conformational disorders, or protein misfolding diseases) may further include such diseases as Creutzfeldt-Jakob disease and other prion diseases, Alzheimer's disease, Parkinson's disease, amyloidosis, multiple system atrophy, and a wide range of other disorders. In some specific embodiments, the proteopathy or protein-misfolding disorder may be Amyloidosis. Thus, in some embodiments, the therapeutic methods of the present disclosure may be applicable for treating amyloidosis, and any related conditions.

Specifically, Amyloidosis is a group of diseases in which abnormal proteins, known as amyloid fibrils, build up in tissue. Symptoms depend on the type and are often variable. They may include diarrhea, weight loss, feeling tired, enlargement of the tongue, bleeding, numbness, feeling faint with standing, swelling of the legs, or enlargement of the spleen.

There are about 30 different types of amyloidosis, each due to a specific protein misfolding. Some are genetic while others are acquired. They are grouped into localized and systemic forms. The four most common types of systemic disease are light chain (AL), inflammation (AA), dialysis (Aβ₂M), and hereditary and old age (ATTR). It should be understood that the CAR molecules, nucleic acid molecules, cells, gene editing system/s, compositions and methods of the present disclosure, may be applicable for any type of amyloidosis, specifically, any type discussed in the present disclosure.

Additional examples of protein misfolding diseases relevant to the methods of the present disclosure may include any disorder that involves directly or indirectly BCMA expression, specifically, overexpression. Such disorders, include but are not limited to Alzheimer's disease, Cerebral β-amyloid angiopathy, Retinal ganglion cell degeneration in glaucoma, Prion diseases (multiple), Parkinson's disease and other synucleinopathies (multiple), Tauopathies (multiple) Frontotemporal lobar degeneration (FTLD), Amyotrophic lateral sclerosis (ALS), Huntington's disease and other trinucleotide repeat disorders (multiple), Familial British dementia, Familial Danish dementia, Hereditary cerebral hemorrhage with amyloidosis (Icelandic) (HCHWA-I), Alexander disease, Pelizaeus-Merzbacher disease, Seipinopathies, Familial amyloidotic neuropathy, Senile systemic amyloidosis, Serpinopathies (multiple), AL (light chain) amyloidosis (primary systemic amyloidosis), AH (heavy chain) amyloidosis, AA (secondary) amyloidosis, Type II diabetes, Aortic medial amyloidosis, ApoAI amyloidosis, ApoAII amyloidosis, ApoAIV amyloidosis, Familial amyloidosis of the Finnish type (FAF), Lysozyme amyloidosis, Fibrinogen amyloidosis, Dialysis amyloidosis, Inclusion body myositis/myopathy, Cataracts, Retinitis pigmentosa with rhodopsin mutations, Medullary thyroid carcinoma, Cardiac atrial amyloidosis, Pituitary prolactinoma, Hereditary lattice corneal dystrophy, Cutaneous lichen amyloidosis, Mallory bodies, Corneal lactoferrin amyloidosis, Pulmonary alveolar proteinosis, Odontogenic (Pindborg) tumor amyloid, Seminal vesicle amyloid, Apolipoprotein C2 amyloidosis, Apolipoprotein C3 amyloidosis, Lect2 amyloidosis, Insulin amyloidosis, Galectin-7 amyloidosis (primary localized cutaneous amyloidosis), Corneodesmosin amyloidosis, Enfuvirtide amyloidosis, Cystic fibrosis, Sickle cell disease.

In yet some further embodiments, since amyloidosis is also classified as a deposition disorder, the methods of the invention may be also applicable for any deposition disorder. Deposition disorder, as used herein is any disorder involving or characterized by deposition of insoluble extracellular protein fragments, or any other metabolite, that have been rendered resistant to digestion, thereby interfering and impairing tissue or organ function and may lead to organ failure.

Still further, as discussed herein above, according to some embodiments, the methods of the invention may be used for the treatment of a patient suffering from any autoimmune disorder. In some specific embodiments, the methods of the invention may be used for treating an autoimmune disease such as for example, but not limited to, systemic lupus erythematosus (SLE), inflammatory bowel disease (IBD), ulcerative colitis, Crohn's disease, fatty liver disease, Lymphocytic colitis, Ischaemic colitis, Diversion colitis, Behçet's syndrome, Indeterminate colitis, rheumatoid arthritis, Graft versus Host Disease (GvHD), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

A further aspect of the present disclosure relates to an effective amount of at least one of:
(a) at least one nucleic acid molecule encoding least one CAR molecule; (b) at least one cassette, vector vehicle or gene editing system comprising said nucleic acid molecule of (a); (c) at least one cell specifically, a cell of the T linage, expressing the CAR molecule, or a population of these cells; and (d) a composition comprising at least one of (a), (b) and (c); for use in a method for treating, preventing, ameliorating, inhibiting or delaying the onset of a of an immune-related disorder in a mammalian subject. In some embodiments of the discussed use, such e CAR molecule comprises the following components. First (i), at least one target-binding domain; wherein at least one of said target binding domain specifically recognizes and binds BCMA; second (ii), at least one hinge and at least one transmembrane domain derived from the CD8α protein. It should be noted that the hinge region of the domain comprises the amino acid sequence as denoted by SEQ ID NO:9. The CAR molecule further comprises as a third component (iii), at least one intracellular T cell signal transduction domain. More specifically, this domain comprises at least one domain of TNF receptor family member, and optionally, at least one domain of a TCR molecule.

In some embodiments, the effective amount for use of the present disclosure may be applicable any of the CAR molecules as defined by the present disclosure. In some alternative or additional embodiments, the use of the present disclosure may be applicable for any of the nucleic acid molecule/s as defined by the present disclosure. In yet some further alternative or additional embodiments, the use of the present disclosure may be applicable for any of the gene editing system/s defined by the present disclosure. In yet some further alternative or additional embodiments, the use of the present disclosure may be applicable any of the cell/s or population of cells as defined by the present disclosure. In yet some further alternative or additional embodiments, the use of the present disclosure may be applicable for any of the compositions defined by the present disclosure.

As described herein above, the invention provides in some aspects thereof therapeutic and prophylactic methods. It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

The term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. The term “amelioration” as referred to herein, relates to a decrease in the symptoms, and improvement in a subject's condition brought about by the compositions and methods according to the invention, wherein said improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with. The term “eliminate” relates to the substantial eradication or removal of the pathologic symptoms and possibly pathologic etiology, optionally, according to the methods of the invention described herein. The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the invention. As indicated above, the methods and compositions provided by the present invention may be used for the treatment of a “pathological disorder”, specifically, immune-related disorders as specified by the invention, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein. It should be appreciated that any of the methods and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms. The present invention relates to the treatment of subjects or patients, in need thereof. By “patient” or “subject in need” it is meant any organism who may be affected by the above-mentioned conditions, and to whom the therapeutic and prophylactic methods herein described are desired, including any vertebrate, specifically mammals such as humans, domestic and non-domestic mammals such as canine and feline subjects, bovine, simian, equine and rodents, specifically, murine subjects. More specifically, the methods of the invention are intended for mammals. By “mammalian subject” is meant any mammal for which the proposed therapy is desired, including human, livestock, equine, canine, and feline subjects, most specifically humans. It should be appreciated that the invention may be applicable for any vertebrates, for example, avian subjects, and fish.

In yet some further aspect, the present disclosure provides a method for targeted activation of a cell of the T lineage against a target cell expressing the BCMA protein and/or a tissue comprising the target cell. More specifically, the method comprising the step of contacting the cell of the T linage with an effective amount of at least one of:
(a) at least one nucleic acid molecule encoding least one CAR molecule; (b) at least one cassette, vector vehicle or gene editing system comprising said nucleic acid molecule of (a); and (c) a composition comprising at least one of (a) and (b); More specifically, such CAR molecule comprises the following components. First (i), at least one target-binding domain; wherein at least one of said target binding domain specifically recognizes and binds BCMA; second (ii), at least one hinge and at least one transmembrane domain derived from the CD8α protein. It should be noted that the hinge region of the domain comprises the amino acid sequence as denoted by SEQ ID NO:9, and any fragments, derivatives and variants thereof. The CAR molecule further comprises as a third component (iii), at least one intracellular T cell signal transduction domain. More specifically, this domain comprises at least one domain of TNF receptor family member, and optionally, at least one domain of a TCR molecule.

In some embodiments of the disclosed methods, the step of contacting the cell/s of the T lineage with the at least one nucleic acid cassette, is performed in vivo, in vitro or ex vivo.

The method of the invention involves the step of contacting the nucleic acid cassette provided by the method of the invention with the target cells. The term “contacting” means to bring, put, incubate or mix together. More specifically, in the context of the present invention, the term “contacting” includes all measures or steps, which allow the positioning of the nucleic acid cassettes of the present invention such that they are in direct or indirect contact with the target cell/s.

To induce DNA integration either in vitro or in vivo, the nucleic acid cassette of the invention may be provided to and/or contacted with the target cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The nucleic acid cassette may be provided to the target cells one or more times, e.g. one time, twice, three times, or more than three times, and the cells allowed to incubate with the nucleic acid cassette for some amount of time following each contacting event e.g. 16-24 hours. Still further, in some embodiments, the contacting step of the cell/s of T lineage with the at least one nucleic acid cassette, is performed in vivo in a subject suffering from at least one immune-related disorder. According to such embodiments, the method further comprises administering to the subject an effective amount of the nucleic acid cassette, a vector comprising said nucleic acid cassette, a gene editing system comprising the nucleic acid molecule, or any composition thereof. In yet some alternative embodiments of the disclosed methods, the step contacting the cell of T lineage with the at least one nucleic acid cassette is performed in vitro or ex vivo to obtain genetically engineered cells of the T lineage, or a population of the cells.

In some embodiments, the method is for targeted activation of a cell of the T lineage against a target cell expressing the BCMA protein and/or a tissue comprising the target cell in a subject suffering from an immune-related disorder. Accordingly, the method further comprises the step of introducing the genetically engineered cells to the subject.

In some embodiments, the cells are of autologous source. In yet some alternative embodiments, the cells are of allogeneic source.

Still further, in some embodiments, the subject is suffering of at least one proliferative disorder, and/or at least one autoimmune disease.

In some embodiments, the methods of the present disclosure may use any of the CAR molecules as defined by the present disclosure. In some alternative or additional embodiments, the methods of the present disclosure may use any of the nucleic acid molecule/s as defined by the present disclosure. In yet some further alternative or additional embodiments, the methods of the present disclosure may use any of the gene editing system/s defined by the present disclosure. In yet some further alternative or additional embodiments, the methods of the present disclosure may use any of the cell/s or population of cells as defined by the present disclosure. In yet some further alternative or additional embodiments, the methods of the present disclosure may use any of the compositions defined by the present disclosure.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Thus, as used herein the term “about” refers to 100%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, and/or parts, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

1-51. (canceled)

52. A chimeric antigen receptor (CAR) molecule comprising:
(i) at least one target-binding domain; wherein at least one of said target binding domain specifically recognizes and binds B cell maturation antigen (BCMA);
(ii) at least one hinge and at least one transmembrane domain derived from the Cluster of Differentiation 8 α (CD8α) protein, wherein said hinge region of said domain comprises the amino acid sequence as denoted by SEQ ID NO:9 and any fragments, derivatives and variants thereof; and
(iii) at least one intracellular T cell signal transduction domain, said domain comprising at least one domain of tumor necrosis factor (TNF) receptor family member, and optionally, at least one domain of a T cell receptor (TCR) molecule.

53. The CAR molecule according to claim 52, wherein at least one of:
(A) said at least one target binding domain comprises: (i) at least one target-recognition element; and/or (ii) at least one adaptor component that recognizes and binds at least one tagged target-recognition element; and
(B) said target-recognition domain and/or element comprises at least one antibody specific for said BCMA, or any antigen-binding fragment/s, portion/s or chimera/s thereof, optionally, at least one of: (a) said antigen-binding fragment/s, portion/s or chimera/s of said antibody comprises at least one of a single chain variable fragment (scFv), and/or nanobody; (b) said antibody specifically recognizes and binds the BCMA protein, or any fragments thereof, said antibody comprising an immunoglobulin heavy chain (HC) comprising the amino acid sequence as denoted by SEQ ID NO: 3, and any derivatives and variants thereof, and an immunoglobulin light chain (LC) comprising the amino acid sequence as denoted by SEQ ID NO: 5, and any derivatives and variants thereof.

54. The CAR molecule according to claim 52, wherein said at least one target-binding domain comprises the amino acid sequence as denoted by SEQ ID NO: 11, and any variants and derivatives thereof.

55. The CAR molecule according to claim 52, wherein at least one of:
(a) said hinge and transmembrane domain comprises the amino acid sequence as denoted by SEQ ID NO: 6, and any variants and derivatives thereof,
(b) said at least one tumor necrosis factor (TNF) receptor family member is the 4-1BB, and/or said TCR molecule comprises a cluster of differentiation 3 (CD3) zeta chain; and
(c) said at least one intracellular T cell signal transduction domain comprises the amino acid sequence as denoted by SEQ ID NO: 12, and any derivatives and variants thereof.

56. The CAR molecule according to claim 52, wherein said CAR comprises the amino acid sequence as denoted by SEQ ID NO: 1, and any variants and derivatives thereof.

57. The CAR molecule according to claim 52, wherein expression of said CAR by at least one cell of the T lineage results in at least one of: (i) increased specificity; (ii) reduced tonic signaling; (iii) reduced off-target activation; (iv) increased expression of activation markers in response to a specific stimulation; and (v) reduced expression of exhaustion markers in response to a specific stimulation, in an in vivo and/or in vitro/ex vivo setting.

58. A nucleic acid molecule comprising at least one nucleic acid sequence encoding at least one CAR molecule as defined in claim 52, or any cassette, vector or vehicle comprising said nucleic acid molecule, said CAR comprising:
(i) at least one target-binding domain; wherein at least one of said target binding domain specifically recognizes and binds BCMA;
(ii) at least one hinge and at least one transmembrane domain derived from the CD8α protein, wherein said hinge region of said domain comprises the amino acid sequence as denoted by SEQ ID NO:9, and any derivatives and variants thereof, and
(iii) at least one intracellular T cell signal transduction domain, said domain comprises at least one domain of TNF receptor family member, and optionally, at least one domain of a TCR molecule; optionally, said nucleic acid molecule is flanked on at least one of the 5′ and 3′ ends thereof by at least one of: (i) homology arms, for integration to a genomic target site by homologous recombination; and/or (ii) recognition sites for a site-specific nuclease, a site-specific integrase or a site-specific recombinase.

59. A gene editing system comprising:
(a) at least one nucleic acid molecule comprising at least one nucleic acid sequence encoding at least one CAR molecule as defined in claim 52, or any cassette, vector or vehicle comprising said nucleic acid molecule, said CAR comprising:
(i) at least one target-binding domain; wherein at least one of said target binding domain specifically recognizes and binds BCMA;
(ii) at least one hinge and at least one transmembrane domain derived from the CD8α protein, wherein said hinge region of said domain comprises the amino acid sequence as denoted by SEQ ID NO:9, and any derivatives and variants thereof, and
(iii) at least one intracellular T cell signal transduction domain, said domain comprises at least one domain of TNF receptor family member, and optionally, at least one domain of a TCR molecule; and
(b) at least one gene editing component or a nucleic acid sequence encoding said gene editing component.

60. A genetically engineered cell of the T cell lineage expressing at least one CAR molecule, or any population of cells comprising at least one said genetically modified cell, said CAR comprising:
(i) at least one target-binding domain; wherein at least one of said target binding domain specifically recognizes and binds BCMA;
(ii) at least one hinge and at least one transmembrane domain derived from the CD8α protein, wherein said hinge region of said domain comprises the amino acid sequence as denoted by SEQ ID NO:9, and any fragments, derivatives and variants thereof; and
(iii) at least one intracellular T cell signal transduction domain, said domain comprising at least one domain of TNF receptor family member, and optionally, at least one domain of a TCR molecule; optionally, at least one of:
(a) said CAR molecule is as defined by claim 52; and
(b) said cell of the T lineage is at least one T cell and/or at least one NK T cell.

61. A composition comprising at least one of: at least one CAR molecule as defined in claim 52, any nucleic acid molecule comprising at least one nucleic acid sequence encoding said CAR molecule, or any cassette, vector, vehicle or gene editing system comprising said nucleic acid molecule, and/or any genetically engineered cell of the T lineage expressing said CAR or population of cells comprising at least one said genetically engineered cell of the T lineage, and any combinations thereof, said CAR comprising:
(i) at least one target-binding domain; wherein at least one of said target binding domain specifically recognizes and binds BCMA;
(ii) at least one hinge and at least one transmembrane domain derived from the CD8α protein, wherein said hinge region of said domain comprises the amino acid sequence as denoted by SEQ ID NO:9, and any fragments, derivatives and variants thereof; and
(iii) at least one intracellular T cell signal transduction domain, said domain comprising at least one domain of TNF receptor family member, and optionally, at least one domain of a TCR molecule;
said composition further comprises at least one of pharmaceutically acceptable carrier/s, diluent/s, excipient/s and additive/s; optionally,

62. A method for treating, preventing, ameliorating, inhibiting or delaying the onset of an immune-related disorder in a mammalian subject, said method comprising the step of administering to said subject an effective amount of at least one of:
(a) at least one nucleic acid molecule encoding least one CAR molecule;
(b) at least one cassette, vector vehicle or gene editing system comprising said nucleic acid molecule of (a);
(c) at least one cell expressing said CAR, or a population of said cells; and
(d) a composition comprising at least one of (a), (b) and (c);
said CAR comprising:
(i) at least one target-binding domain; wherein at least one of said target binding domain specifically recognizes and binds BCMA;
(ii) at least one hinge and at least one transmembrane domain derived from the CD8α protein, wherein said hinge region of said domain comprises the amino acid sequence as denoted by SEQ ID NO:9, and any fragments, derivatives and variants thereof; and
(iii) at least one intracellular T cell signal transduction domain, said domain comprises at least one domain of TNF receptor family member, and optionally, at least one domain of a TCR molecule.

63. The method according to claim 62, wherein at least one of:
(A) said at least one target binding domain comprises: (i) at least one target-recognition element; and/or (ii) at least one adaptor component that recognizes and binds at least one tagged target-recognition element;
(B) said target-recognition domain and/or element comprises at least one antibody specific for said BCMA, or any antigen-binding fragment/s, portion/s or chimera/s thereof; optionally, at least one of:
(a) said antigen-binding fragment/s, portion/s or chimera/s of said antibody comprises at least one of a scFv, and/or a nanobody;
(b) said antibody specifically recognizes and binds the BCMA protein, said antibody comprising an immunoglobulin HC comprising the amino acid sequence as denoted by SEQ ID NO: 3, and any derivatives and variants thereof, and an immunoglobulin LC comprising the amino acid sequence as denoted by SEQ ID NO: 5, and any derivatives and variants thereof;
(C) said at least one target-binding domain comprises the amino acid sequence as denoted by SEQ ID NO: 11, and any derivatives and variants thereof;
(D) said hinge and transmembrane domain comprises the amino acid sequence as denoted by SEQ ID NO: 6, and any derivatives and variants thereof;
(E) said at least one TNF receptor family member is the 4-1BB, and wherein said TCR molecule comprises a CD3 zeta chain; and
(F) said at least one intracellular T cell signal transduction domain comprises the amino acid sequence as denoted by SEQ ID NO: 12.

64. The method according to claim 62, wherein said CAR comprises the amino acid sequence as denoted by SEQ ID NO: 1, or any variants and derivatives thereof.

65. The method according to claim 62, wherein expression of said CAR by at least one cell of the T lineage of said subject results in at least one of: (i) increased specificity; (ii) reduced tonic signaling; (iii) reduced off-target activation; (iv) increased expression of activation markers in response to a specific stimulation; (v) reduced expression of exhaustion markers in response to a specific stimulation; (vi) increased survival; (vii) reduced relapse rate; and (viii) long-term effect; in said subject;

66. The method according to claim 62, wherein at least one of:
(a) said subject is administered with at least one cell of the T lineage expressing said CAR molecule, and/or genetically engineered with said at least one nucleic acid cassette or any vector or vehicle comprising said cassette or with a population of said cells;
(b) said cells are of an autologous or allogeneic source; and
(c) said subject is administered with a nucleic acid vector comprising said at least one cassette, said vector is any one of a viral vector, a non-viral vector and a naked DNA vector.

67. The method according to claim 62, wherein said immune-related disorder is a disorder associated with expression of the BCMA protein in cells of the B lineage.

68. The method according to claim 67, wherein at least one of:
(a) said disorder is at least one of: at least one proliferative disorder, and/or at least one autoimmune disease, a deposition disorder, or any B cell-mediated disorder
(b) said proliferative disorder is any B cell malignancy;
(c) said B cell malignancy is multiple myeloma (MM) and any related conditions; and
(d) said deposition disorder is amyloidosis, and any related conditions.

69. A method for targeted activation of a cell of the T lineage against a target cell expressing the BCMA protein and/or a tissue comprising said target cell, the method comprising the step of contacting said cell of the T lineage with an effective amount of at least one of:
(A) at least one nucleic acid molecule encoding least one CAR molecule as defined in claim 52;
(B) at least one cassette, vector vehicle or gene editing system comprising said nucleic acid molecule of (a); and
(C) a composition comprising at least one of (A) and (B); wherein said CAR comprising:
(i) at least one target-binding domain; wherein at least one of said target binding domain specifically recognizes and binds BCMA;
(ii) at least one hinge and at least one transmembrane domain derived from the CD8α protein, wherein said hinge region of said domain comprises the amino acid sequence as denoted by SEQ ID NO:9, and any fragments, derivatives and variants thereof; and
(iii) at least one intracellular T cell signal transduction domain, said domain comprises at least one domain of TNF receptor family member, and optionally, at least one domain of a TCR molecule.

70. The method according to claim 69, wherein at least one of:
(a) the step of contacting said cell of the T lineage with said at least one nucleic acid cassette, is performed in vivo, in vitro or ex vivo;
(b) wherein contacting said cell of T lineage with said at least one nucleic acid cassette, is performed in vivo in a subject suffering from at least one immune-related disorder, the method further comprising administering to said subject an effective amount of said nucleic acid cassette, a vector comprising said nucleic acid cassette, a gene editing system comprising said nucleic acid molecule, or any composition thereof,
(c) wherein contacting said cell of T lineage with said at least one nucleic acid cassette is performed in vitro or ex vivo to obtain genetically engineered cells of the T lineage, or a population of said cells.

71. The method according to claim 70, wherein at least one of:
(a) the method is for targeted activation of a cell of the T lineage against a target cell expressing the BCMA protein and/or a tissue comprising said target cell in a subject suffering from an immune-related disorder, and wherein said method further comprises the step of introducing said genetically engineered cells to said subject;
(b) said cells of the T lineage are of autologous or allogeneic source; and
(c) said disorder is at least one of: at least one proliferative disorder, at least one deposition disorder and/or at least one autoimmune disease.


SEQUENCE LISTING (40 sequences)

SEQ ID NO: 1 - protein, 488 aa, synthetic construct
  source 1..488: note = H8BB CAR
MALPVTALLLPLALLLHAARPQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 21 aa, synthetic construct
  source 1..21: note = H8BB CAR - CD8
MALPVTALLLPLALLLHAARP

SEQ ID NO: 3 - protein, 117 aa, synthetic construct
  source 1..117: note = H8BB CAR - HC c11d5.3
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 4 - protein, 15 aa, synthetic construct
  source 1..15: note = H8BB CAR -LINKER
GGGGSGGGGSGGGGS

SEQ ID NO: 5 - protein, 111 aa, synthetic construct
  source 1..111: note = H8BB CAR LC c11d5.3
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 6 - protein, 70 aa, synthetic construct
  source 1..70: note = H8BB CAR CD8A hinge/TM domain
PTTTPAPRPPTPAPTIASQPLSLRPEACRPAAGGAVHTRGLDFACDIYIWAPLAGTCGVLLLSLVITLYC

SEQ ID NO: 7 - protein, 42 aa, synthetic construct
  source 1..42: note = H8BB CAR 41BB
KRGRKKLLYIFKQPFMRPVQTTQEEDGCSCRFPEEEEGGCEL

SEQ ID NO: 8 - protein, 112 aa, synthetic construct
  source 1..112: note = H8BB CAR CD3z
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 46 aa, synthetic construct
  source 1..46: note = H8BB CAR Hinge
PTTTPAPRPPTPAPTIASQPLSLRPEACRPAAGGAVHTRGLDFACD

SEQ ID NO: 10 - protein, 24 aa, synthetic construct
  source 1..24: note = H8BB CAR TM
IYIWAPLAGTCGVLLLSLVITLYC

SEQ ID NO: 11 - protein, 264 aa, synthetic construct
  source 1..264: note = H8BB CAR extracellular domain
MALPVTALLLPLALLLHAARPQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 12 - protein, 154 aa, synthetic construct
  source 1..154: note = H8BB CAR intracellular domain
KRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 7018 bp, synthetic construct
  source 1..7018: mol_type = other DNA; note = BCMA CAR H8BB; plasmid = Plasmid_MS6VA
atggcactgcctgtgaccgccctgctgctgccactggccctgctgctgcacgcagcccggccccagatccagctggtgcagtccggccccgagctgaagaagcctggcgagacagtgaagatctcctgtaaggcctctggctacaccttcacagactattctatcaactgggtgaagagggcaccaggcaagggcctgaagtggatgggctggatcaataccgagacacgggagcccgcctacgcctatgacttcagaggcaggttcgcctttagcctggagacctctgccagcacagcctacctgcagatcaacaatctgaagtacgaggataccgccacatatttttgtgccctggactactcctatgctatggactattggggccagggcacctctgtgacagtgagctccggaggaggaggaagcggcggaggaggctccggcggcggcggctctgatatcgtgctgacccagtctccccctagcctggccatgagcctgggcaagagagccaccatctcctgcagggcctccgagtctgtgaccattttgggttcccatctcatccactggtatcagcagaaacccggtcaaccccctacattgctgatccagctggcctccaacgtgcagacaggagtgcctgcacgcttcagcggatccggatctcggaccgactttaccctgacaatcgatccagtggaggaggacgatgtggccgtgtactattgcctgcagtccagaaccatccccaggacatttggcggcggcacaaagctggagatcaagcccaccacaacccctgcaccacgcccacccacaccagcacctaccatcgcaagccagccactgtccctgcggcccgaggcctgtaggccagcagcaggaggagcagtgcacaccaggggcctggactttgcctgcgatatctacatctgggcaccactggcaggaacatgtggcgtgctgctgctgagcctggtcatcaccctgtattgcaagagaggcaggaagaagctgctgtacatcttcaagcagcccttcatgcggcccgtgcagacaacccaggaggaggacggctgctcctgtaggttccctgaagaggaggagggaggatgtgagctgagggtgaagttttcccggtctgccgatgccccagcctataagcagggccagaaccagctgtacaacgagctgaatctgggccggagagaggagtatgacgtgctggataagaggcgcggcagggaccccgagatgggaggcaagccccggagaaagaaccctcaggagggcctgtataatgagctgcagaaggacaagatggccgaggcctacagcgagatcggcatgaagggagagaggcgccggggcaagggacacgatggcctgtaccagggcctgtccacagcaaccaaagacacttacgacgcactgcacatgcaggctctgccaccaagataagcggccgctacgtagtcgacggatccctcgagctcaagcttcgaattctgcagtcgacggtaccgcgggcccgggatccgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatccatcgatggagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagaccgcc

SEQ ID NO: 14 - DNA, 63 bp, synthetic construct
  source 1..63: mol_type = other DNA; note = BCMA CAR H8BB CD8 leader
atggcactgcctgtgaccgccctgctgctgccactggccctgctgctgcacgcagcccggccc

SEQ ID NO: 15 - DNA, 351 bp, synthetic construct
  source 1..351: mol_type = other DNA; note = BCMA CAR H8BB -Heavy Chain c11d5.3
cagatccagctggtgcagtccggccccgagctgaagaagcctggcgagacagtgaagatctcctgtaaggcctctggctacaccttcacagactattctatcaactgggtgaagagggcaccaggcaagggcctgaagtggatgggctggatcaataccgagacacgggagcccgcctacgcctatgacttcagaggcaggttcgcctttagcctggagacctctgccagcacagcctacctgcagatcaacaatctgaagtacgaggataccgccacatatttttgtgccctggactactcctatgctatggactattggggccagggcacctctgtgacagtgagctcc

SEQ ID NO: 16 - DNA, 45 bp, synthetic construct
  source 1..45: mol_type = other DNA; note = BCMA CAR H8BB (G4S)3 linker
ggaggaggaggaagcggcggaggaggctccggcggcggcggctct

SEQ ID NO: 17 - DNA, 333 bp, synthetic construct
  source 1..333: mol_type = other DNA; note = BCMA CAR H8BB Light Chain c11d5.3
gatatcgtgctgacccagtctccccctagcctggccatgagcctgggcaagagagccaccatctcctgcagggcctccgagtctgtgaccattttgggttcccatctcatccactggtatcagcagaaacccggtcaaccccctacattgctgatccagctggcctccaacgtgcagacaggagtgcctgcacgcttcagcggatccggatctcggaccgactttaccctgacaatcgatccagtggaggaggacgatgtggccgtgtactattgcctgcagtccagaaccatccccaggacatttggcggcggcacaaagctggagatcaag

SEQ ID NO: 18 - DNA, 210 bp, synthetic construct
  source 1..210: mol_type = other DNA; note = BCMA CAR H8BB - CD8
cccaccacaacccctgcaccacgcccacccacaccagcacctaccatcgcaagccagccactgtccctgcggcccgaggcctgtaggccagcagcaggaggagcagtgcacaccaggggcctggactttgcctgcgatatctacatctgggcaccactggcaggaacatgtggcgtgctgctgctgagcctggtcatcaccctgtattgc

SEQ ID NO: 19 - DNA, 126 bp, synthetic construct
  source 1..126: mol_type = other DNA; note = BCMA CAR H8BB -41BB
aagagaggcaggaagaagctgctgtacatcttcaagcagcccttcatgcggcccgtgcagacaacccaggaggaggacggctgctcctgtaggttccctgaagaggaggagggaggatgtgagctg

SEQ ID NO: 20 - DNA, 339 bp, synthetic construct
  source 1..339: mol_type = other DNA; note = BCMA CAR H8BB - CD3z
agggtgaagttttcccggtctgccgatgccccagcctataagcagggccagaaccagctgtacaacgagctgaatctgggccggagagaggagtatgacgtgctggataagaggcgcggcagggaccccgagatgggaggcaagccccggagaaagaaccctcaggagggcctgtataatgagctgcagaaggacaagatggccgaggcctacagcgagatcggcatgaagggagagaggcgccggggcaagggacacgatggcctgtaccagggcctgtccacagcaaccaaagacacttacgacgcactgcacatgcaggctctgccaccaagataa

SEQ ID NO: 21 - protein, 503 aa, synthetic construct
  source 1..503: note = H828 CAR
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 55 aa, synthetic construct
  source 1..55: note = H828 CAR Kochenderfer Hinge
FVPVFLPAKPTTTPAPRPPTPAPTIASQPLSLRPEACRPAAGGAVHTRGLDFACD

SEQ ID NO: 23 - protein, 21 aa, synthetic construct
  source 1..21: note = H828 CAR Kochenderfer TM
IYIWAPLAGTCGVLLLSLVIT

SEQ ID NO: 24 - protein, 8 aa, synthetic construct
  source 1..8: note = H828 CAR Kochenderfer IC
LYCNHRNR

SEQ ID NO: 25 - protein, 184 aa, Homo sapiens
  source 1..184: note = BCMA
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 26 - protein, 235 aa, Homo sapiens
  source 1..235: note = CD8 alpha UniProt accession number P01732-1
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 27 - protein, 164 aa, Homo sapiens
  source 1..164: note = CD3 Zeta UniProt accession number P20963-1
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 255 aa, Homo sapiens
  source 1..255: note = 4-1BB UniProt accession number Q07011
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 29 - DNA, 19 bp, synthetic construct
  source 1..19: mol_type = other DNA; note = MSGV1 primer Forward
cggcagcctaccaagaaca

SEQ ID NO: 30 - DNA, 19 bp, synthetic construct
  source 1..19: mol_type = other DNA; note = MSGV1 reverse primer
tgtgtcgccgactcggtaa

SEQ ID NO: 31 - DNA, 15 bp, synthetic construct
  source 1..15: mol_type = other DNA; note = MSGV1 probe
cggtggtacctcacc

SEQ ID NO: 32 - DNA, 22 bp, synthetic construct
  source 1..22: mol_type = other DNA; note = Albumin primer Forward
gagtcaccaaatgctgcacaga

SEQ ID NO: 33 - DNA, 17 bp, synthetic construct
  source 1..17: mol_type = other DNA; note = Albumin reverse primer
gaacgtatgtttcatcg

SEQ ID NO: 34 - DNA, 15 bp, synthetic construct
  source 1..15: mol_type = other DNA; note = Albumin probe
acaggcgaccatgct

SEQ ID NO: 35 - protein, 47 aa, synthetic construct
  source 1..47: note = H8BB CAR Hinge
KPTTTPAPRPPTPAPTIASQPLSLRPEACRPAAGGAVHTRGLDFACD

SEQ ID NO: 36 - protein, 466 aa, synthetic construct
  source 1..466: note = ICBB- CAR
MALPVTALLLPLALLLHAARPQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKNWSHPQFEKESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 465 aa, synthetic construct
  source 1..465: note = IC28 CAR
MALPVTALLLPLALLLHAARPQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKNWSHPQFEKESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 21 aa, synthetic construct
  source 1..21: note = Hinge IgG4-Stag
NWSHPQFEKESKYGPPCPPCP

SEQ ID NO: 39 - protein, 26 aa, synthetic construct
  source 1..26: note = TMCD28
FWVLVVVGGVLACYSLLVTVAFIIFW

SEQ ID NO: 40 - protein, 467 aa, synthetic construct
  source 1..467: note = H8BB CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR